(19) United States
(12) Patent Application Publication
(10) Pub. No.: US 2005/0221303 A1
(43) Pub. Date: Oct. 6, 2005

(54) Sequences involved in phenomena of tumour suppression, tumour reversion, apoptosis and/or virus resistance and their use as medicines

(76) Inventors: Adam Telerman, Paris (FR); Robert Amson, Paris (FR); Marius Tuijnder, Hombeek (BE); Laurent Susini, Montmorency (FR)

Correspondence Address: GLENDA C CALLAHAN, 413 JEAN STREET, HOUMA, LA, 70360, US

(21) Appl. No.: 10/467,851

(22) PCT Filed: Feb. 13, 2002
(86) PCT No.: PCT/FR02/00543

§ 371 Date: Feb. 9, 2005

(30) Foreign Application Priority Data
Feb. 13, 2001 (FR) 01/01925

(51) Int. Cl. C12Q 1/68; C07H 21/04; C12N 15/09; C12N 9/99

(52) U.S. Cl. 435/6; 435/69.2; 435/184; 435/320.1; 435/325; 536/23.2

(57) ABSTRACT

This invention is directed to sequences that are involved in the molecular pathways of tumor suppression, tumor revision, apoptosis and/or virus resistance. The use of the compounds having these sequences, or encoded by them, in treating cancer, neurodegenerative diseases or viral diseases, and screening methods for compounds having such therapeutic properties are also encompassed.

The present invention relates to the identification of genes involved in the molecular pathways of tumour suppression, tumour reversion, apoptosis and/or resistance to viruses.

The present invention was made possible by the isolation of cDNA corresponding to messenger RNAs expressed or repressed during tumour suppression, tumour reversion and/or during the process of apoptosis.

In order to isolate the genes activated or inhibited during tumour reversion, overall screening for the expression of the genes in a malignant cell line (U937) and a derived cell line (US4) was carried out with suppression of the malignant phenotype. Comparison of the expressed genes (messenger RNAs expressed in both types of cell) made it possible to identify genes expressed differentially, that is to say expressed in one of the cells while they are not in the other (the genes may be activated or inhibited).

It is easily deduced therefrom that these genes are at least involved in the cancerization process, in one case by their absence, and in the other case by their presence.

For this differential study, the method used is the method described in 2000 by Brenner et al. (Proc. Natl. Acad. Sci. USA, 97 (4), 1665-70).

In order to produce a model, the inventors made the following hypotheses: if it were possible to select, from a tumour which is sensitive to the cytopathic effect of the parvovirus H-1, cells which are resistant, then this resistance could be due to a change in their malignant phenotype. It has been possible to demonstrate this for US4 cells selected from the U937 cancer cells. Unlike the U937 parental line, the US4 clones (but also US3 clones which will not be dealt with in the present invention) are resistant to the cytopathic effect of the parvovirus H-1.

At the molecular level, it has been possible to observe that this suppression of the malignant phenotype was accompanied by activation of the expression of the p21^{waf1} gene, this being independently of the expression of the p53 gene.

The approach to the problem according to the present invention made it possible to isolate sequences directly linked to several precise functions. Accordingly, unlike the random sequencing of ESTs, the sequences are sequences whose function is known since they are involved in the process of suppression of the malignant phenotype, of tumour reversion, of apoptosis and/or in resistance to viruses.

Tumour reversion is distinguishable from tumour suppression by the fact that it covers a broader domain than that of tumour suppressor genes. In other words, tumour reversion is achieved by the use of metabolic and/or molecular pathways which are not limited to the metabolic and molecular pathways in which the tumour suppressor genes are involved.

Thus, the present invention relates in particular to novel sequences and to the use of these sequences in the diagnostic field and for carrying out methods for screening test compounds. The invention also relates to methods for the detection and/or assay of the sequences of the invention or of their product(s) of expression in a biological sample.

The present invention first of all relates to an isolated nucleotide sequence comprising a nucleotide sequence chosen from the group comprising:
a) SEQ ID No. 1 to SEQ ID No. 1020, preferably SEQ ID No. 72,
b) a nucleotide sequence of at least 15 consecutive nucleotides of a sequence as defined in a),
c) a nucleotide sequence exhibiting a percentage identity of at least 80%, after optimum alignment, with a sequence defined in a) or b),
d) a nucleotide sequence which hybridizes under high stringency conditions with a sequence defined in a) or b), and
e) a complementary nucleotide sequence where the RNA sequence corresponding to a sequence as defined in a), b), c) or d).

The nucleotide sequence according to the invention defined in c) exhibits a percentage identity of at least 80% after optimum alignment with a sequence as defined in a) or b) above, preferably of at least 90%, more preferably of at least 98%.

In the context of the present invention, SEQ ID No. 72 is the preferred nucleotide sequence and corresponds to the TPT1 gene also called TCTP. This gene is involved in particular in the phenomenon of tumour reversion and has been the subject of more thorough experiments on the part of the inventors as indicated below.

The expressions nucleotide sequence, nucleic acid, nucleic or nucleic acid sequence, polynucleotide, oligonucleotide and polynucleotide sequence, terms which will be used without distinction in the present description, are understood to mean a precise succession of nucleotides, modified or unmodified, which makes it possible to define a fragment or a region of a nucleic acid, containing, or otherwise, unnatural nucleotides, and being capable of corresponding at the same time to a double-stranded DNA, a single-stranded DNA and transcriptional products of the said DNAs. Thus, the nucleic sequences according to the invention also cover PNAs (Peptide Nucleic Acids), or the like.

The fragments of the nucleotide sequences of the invention comprise at least 15 consecutive nucleotides. Preferably, they comprise at least 20 consecutive nucleotides and still more preferably they comprise at least 30 consecutive nucleotides.

It should be understood that the present invention does not relate to the nucleotide sequences in their natural chromosomal environment, that is to say in the natural state. It involves sequences which have been isolated and/or purified, that is to say that they were collected directly or indirectly, for example by copying, their environment having been at least partially modified. The nucleic acids obtained by chemical synthesis are understood to also be thereby designated.

The expression “percentage identity” between two nucleic acid or amino acid sequences for the purposes of the present invention is understood to mean a percentage of identical nucleotides or amino acid residues between the two sequences to be compared, which is obtained after the best alignment, this percentage being purely statistical and the differences between the two sequences being randomly distributed and over their entire length. The expression “best alignment” or “optimum alignment” is understood to mean the alignment for which the percentage identity determined as below is the highest. Sequence comparisons between two nucleic acid or amino acid sequences are traditionally carried out by comparing these sequences after having optimally aligned them, the said comparison being carried out per segment or per “comparison window” in order to identify and compare the local regions with sequence similarity. Optimum alignment of the sequences for comparison may be carried out, apart from manually, by means of the Smith and Waterman local homology algorithm (1981), by means of the Neddleman and Wunsch local homology algorithm (1970), by means of the Pearson and Lipman search for similarity methods (1988), by means of computer software packages using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain optimum alignment, the BLAST program is preferably used, with the BLOSUM 62 matrix. The PAM or PAM 250 matrices can also be used.

The percentage identity between two nucleic acid or amino acid sequences is determined by comparing these two sequences aligned optimally, it being possible for the nucleic acid or amino acid sequence to be compared to comprise additions or deletions in relation to the reference sequence for an optimum alignment between these two sequences. The percentage identity is calculated by determining the number of identical positions for which the nucleotide or amino acid residue is identical between the two sequences, by dividing this number of identical positions by the total number of positions compared and by multiplying the result obtained by 100 in order to obtain the percentage identity between these two sequences.

The expression nucleic sequences exhibiting a percentage identity of at least 80%, preferably of at least 90%, more preferably of at least 98%, after optimum alignment with a reference sequence, is understood to mean the nucleic sequences exhibiting, in relation to the reference nucleic sequence, certain modifications such as in particular a deletion, truncation, alignment, chimeric fusion and/or substitution, in particular point substitution, and whose nucleic sequence exhibits at least 80%, preferably at least 90%, more preferably at least 98%, identity after optimum alignment with the reference nucleic sequence. Preferably, the specific or high stringency hybridization conditions will be such that they ensure at least 80%, preferably at least 90%, more preferably at least 98% identity after optimum alignment between one of the two sequences and the sequence complementary to the other.

Hybridization under high stringency conditions means that the temperature and ionic strength conditions are chosen so that they allow hybridization to be maintained between two complementary nucleic acid fragments. By way of illustration, high stringency conditions of the hybridization step for the purposes of defining the nucleotide sequences described above, will be advantageously the following.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution containing 0.15 M NaCl+0.015 M sodium citrate), 50% formamide, 7% sodium dodecyl sulphate (SDS), 10×Denhardt's, 5% dextran sulphate and 1% salmon sperm DNA; (2) hybridization proper for 20 hours at a temperature dependent on the size of the probe (i.e.: 42° C., for a probe of size>100 nucleotides) followed by 2 washes of 20 minutes at 20° C. in 2×SSC+2% SDS, 1 wash of 20 minutes at 20° C. in 0.1×SSC+0.1% SDS. The final wash is performed in 0.1×SSC+0.1% SDS for 30 minutes at 60° C. for a probe of size>100 nucleotides. The high stringency hybridization conditions described above for a polynucleotide of defined size may be adjusted by persons skilled in the art for larger- or smaller-size oligonucleotides, according to the teaching of Sambrook et al., 1989.

Among the nucleotide sequences exhibiting a percentage identity of at least 80%, preferably of at least 90%, more preferably of at least 98%, after optimum alignment with the sequences according to the invention, preference is also given to the variant nucleic sequences of the sequences of the invention, or of fragments thereof, that is to say all the nucleic sequences corresponding to allelic variants, that is to say individual variations of the sequences of the invention.

The expression “variant nucleotide sequence” is understood to mean any RNA or cDNA resulting from a mutation and/or variation of a splice site of the genomic DNA corresponding to the nucleotide sequences of the invention.

The subject of the present invention is also a polypeptide encoded by a nucleotide sequence in accordance with the invention.

The expression “polypeptide” is understood to mean, for the purposes of the present invention, either proteins or peptides.

According to a particular embodiment, the polypeptides in accordance with the invention comprise a polypeptide chosen from:
a) a polypeptide encoded by a nucleotide sequence in accordance with the invention,
b) a polypeptide exhibiting at least 80% identity with a polypeptide as defined in a),
c) a fragment of at least 5 amino acids of a polypeptide as defined in a) orb),
d) a biologically active fragment of a polypeptide as defined in a), b) or c), and
e) a modified polypeptide of a polypeptide as defined in a), b), c) or d).

The percentage identity is understood to be evaluated after optimum alignment of the relevant sequences. The expression polypeptide whose amino acid sequence exhibits a percentage identity of at least 80%, preferably of at least 90%, more preferably of at least 98% after optimum alignment with a reference sequence, is understood to mean the polypeptides exhibiting certain modifications in relation to the reference polypeptide, such as in particular one or more deletions or truncations, an extension, a chimeric fusion and/or one or more substitutions.

Among the polypeptides whose amino acid sequence exhibits a percentage identity of at least 80%, preferably of at least 90% and more preferably of at least 98%, after optimum alignment with a reference sequence such as a polypeptide in accordance with the present invention or with one of its fragments, preference is given to the variant polypeptides encoded by the variant nucleotide sequences as previously defined, in particular the polypeptides whose amino acid sequence exhibits at least one mutation corresponding in particular to a truncation, deletion, substitution and/or addition of at least one residue in relation to the polypeptide sequences of the invention or with one of their fragments.

The present invention also relates to a cell cloning and/or expression vector, characterized in that it comprises a nucleotide sequence according to the invention or that it encodes a polypeptide according to the invention. Such a vector may also contain the elements necessary for the expression and possibly for the secretion of the polypeptide in a host cell. Such a host cell is also a subject of the present invention.

The vectors comprising promoter and/or regulatory sequences also form part of the present invention. The said vectors preferably contain a promoter, signals for initiation and termination of translation, and appropriate regions for regulation of transcription. They should be able to be stably maintained in the cell and they may also possess particular signals so as to allow the secretion of the translated protein.

These different control signals are chosen according to the cellular host used. To this effect, the nucleic acid sequences according to the invention may be inserted into vectors which replicate autonomously in the chosen host or into integrating vectors of the chosen host.

Among the autonomously replicating systems, use is preferably made, according to the host cell, of the plasmid or viral type systems, it being possible for the viral vectors in particular to be adenoviruses (5), retroviruses, lentiviruses, poxviruses or herpesviruses (5a). Persons skilled in the art know the technologies which can be used for each of these systems.

When the integration of the sequence into the chromosomes of the host cell is desired, it is possible to use, for example, systems of the plasmid or viral type; such viruses are, for example, retroviruses (6) or AAVs (7).

Advantageously, the vectors in accordance with the invention contain a sequence allowing tissue-specific targeting and/or expression.

Among the nonviral vectors, preference is given to naked polynucleotides such as naked DNA or naked RNA according to the technique developed by the company VICAL, bacterial artificial chromosomes (BAC), yeast artificial chromosomes (YAC) for expression in yeast, mouse artificial chromosomes (MAC) for expression in murine cells and preferably human artificial chromosomes (HAC) for expression in human cells.

Such vectors are prepared according to methods commonly used by persons skilled in the art, and the clones resulting therefrom may be introduced into an appropriate host by standard methods such as for example lipofection, electroporation, heat shock, transformation after chemical permeabilization of the membrane, cell fusion.

The invention additionally comprises the host cells, in particular the eukaryotic and prokaryotic cells, transformed with the vectors according to the invention and the transgenic animals, preferably the mammals, except humans, comprising one of the said transformed cells according to the invention. These animals may be used as models, for studying the aetiology of inflammatory and/or immune diseases, and in particular inflammatory diseases of the digestive tube, or for studying cancers.

Among the cells which can be used for the purposes of the present invention, there may be mentioned bacterial cells (8), but also yeast cells (9), as well as animal cells, in particular mammalian cell cultures (10), and in particular Chinese hamster ovary (CHO) cells. There may also be mentioned insect cells in which it is possible to use methods using for example baculoviruses (11). A preferred cellular host for the expression of the proteins of the invention consists of COS cells.

Among the mammals according to the invention, preference is given to animals such as rodents, in particular mice, rats or rabbits, expressing a polypeptide according to the invention.

These transgenic animals are obtained, for example, by homologous recombination on embryonic stem cells, transfer of these stem cells into embryos, selection of the chimeras affected at the level of the reproductive lines, and growth of the said chimeras.

The transgenic animals according to the invention can thus overexpress the gene encoding the protein according to the invention, or their homologous gene, or may express the said gene into which a mutation is introduced. These transgenic animals, in particular mice, are obtained for example by transfection of a copy of this gene under the control of a strong promoter of a ubiquitous nature, or which is selective for a type of tissue, or after viral transcription.

The cells and mammals according to the invention can be used in a method for producing a polypeptide according to the invention, as described below, and can likewise serve as a model for analysis.

The transformed cells or mammals as described above may also be used as models in order to study the interactions between the polypeptides according to the invention and the chemical or protein compounds involved directly or indirectly in the activities of the polypeptides according to the invention, this being in order to study the different mechanisms and interactions involved.

They can in particular be used for the selection of products interacting with the polypeptides according to the invention, or their [lacuna] as a cofactor, or as an inhibitor, in particular competitive, or having an agonist or antagonist activity on the activity of the polypeptides according to the invention.

Preferably, the said transformed cells or transgenic animals are used as a model in particular for the selection of products which make it possible to control pathologies linked to abnormal expression of this gene.

The subject of the present invention is also a monoclonal or polyclonal antibody, a fragment of this antibody or a chimeric antibody capable of specifically recognizing a polypeptide in accordance with the present invention.

The specific monoclonal antibodies may be obtained according to the conventional hybridoma culture method well known to persons skilled in the art.

The antibodies according to the invention are for example humanized antibodies, Fab or F(ab′)² fragments. They may also be provided in the form of immunoconjugates or antibodies labelled so as to obtain a detectable and/or quantifiable signal.

The antibodies in accordance with the invention, but also the immunoconjugates, are therefore capable of specifically recognizing a polypeptide according to the present invention.

The specific polyclonal antibodies may be obtained from the serum of an animal immunized against the polypeptides of the invention, in particular produced by genetic recombination or by peptide synthesis, according to the customary procedures.

The importance of antibodies specifically recognizing the polypeptides, their variants or their immunogenic fragments, according to the invention,: should be noted in particular.

The subject of the invention is also the use of a nucleotide sequence in accordance with the present invention as a probe or a primer for the detection, identification, assay and/or amplification of nucleic acid sequences.

According to the invention, the nucleotide sequences which may be used as a probe or as a primer in methods for the detection, identification, assay and/or amplification of a nucleic acid sequence have a minimum size of 15 bases, preferably of 20 bases, or even better from 25 to 30 bases.

The probes and primers according to the invention may be directly or indirectly labelled with a radioactive or nonradioactive compound by methods well known to persons skilled in the art, so as to obtain a detectable and/or quantifiable signal.

The nonlabelled nucleic sequences according to the invention may be used directly as a probe or a primer.

The sequences are generally labelled in order to obtain sequences which can be used for numerous applications. The labelling of the primers or of the probes according to the invention is carried out with radioactive elements or with nonradioactive molecules.

Among the radioactive isotopes used, there may be mentioned ³²P, ³³P, ³⁵S, ³H or ¹²⁵I. The nonradioactive entities are selected from ligands such as biotin, avidin, streptavidin, dioxygenin, haptens, colourings, luminescent agents such as radioluminescent, chemoluminescent, bioluminescent, fluorescent and phosphorescent agents.

The nucleotide sequences according to the invention may thus be used as a primer and/or a probe in methods using in particular the PCR (polymerase chain reaction) technique (11a). This technique requires the choice of pairs of oligonucleotide primers flanking the fragment which has to be amplified. Reference may be made, for example, to the technique described in American Patent U.S. Pat. No. 4,683,202. The amplified fragments may be identified, for example after agarose or polyacrylamide gel electrophoresis, or after a chromatography technique such as gel filtration or ion-exchange chromatography, and then sequenced. The specificity of the amplification may be checked, using as primers, the nucleotide sequences of the invention and, as templates, plasmids containing these sequences or the derived products of amplification. The amplified nucleotide fragments may be used as reagents in hybridization reactions in order to demonstrate the presence, in a biological sample, of a target nucleic acid having a sequence complementary to that of the said amplified nucleotide fragments.

The invention also relates to the nucleic acids which can be obtained by amplification using primers according to the invention.

Other techniques for amplifying the target nucleic acid may be advantageously used as an alternative to PCR (PCR-like) using pairs of primers of nucleotide sequences according to the invention. The expression PCR-like is understood to mean any of the methods using direct or indirect reproductions of the nucleic acid sequences, or in which the labelling systems were amplified, these techniques are of course known. In general, they involve the amplification of DNA by a polymerase, when the original sample is an RNA, a reverse transcription should be carried out beforehand. A great number of methods allowing this amplification currently exist, such as for example the SDA (Strand Displacement Amplification) technique (12), the TAS (Transcription-based Amplification System) technique described by (13), the 3SR (Self-Sustained Sequence Replication) technique described by (14), the NASBA (Nucleic Acid Sequence Based Amplification) technique described by (15), the TMA (Transcription Mediated Amplification) technique, the LCR (Ligase Chain Reaction) technique described by (16), the RCR (Repair Chain Reaction) technique described by (17), the CPR (Cycling Probing Reaction) technique described by (18), the Q-beta-replicase amplification technique described by (19). Some of these techniques have since been improved.

In the case where the target polynucleotide to be detected is an mRNA, an enzyme of the reverse transcriptase type is advantageously used, prior to the use of an amplification reaction with the aid of the primers according to the invention or to the use of a method of detection with the aid of the probes of the invention, in order to obtain a cDNA from the mRNA contained in the biological sample. The cDNA obtained will then serve as a target for the primers or probes used in the method of amplification or detection according to the invention.

The probe hybridization technique may be carried out in various ways (20). The method most generally used consists in immobilizing the nucleic acid extracted from the cells of different tissues or cells in culture on a support (such as nitrocellulose, nylon, polystyrene) and in incubating, under well defined conditions, the immobilized target nucleic acid with the probe. After hybridization, the excess probe is removed and the hybrid molecules formed are detected by the appropriate method (measurement of the radioactivity, the fluorescence or the enzymatic activity linked to the probe).

According to another embodiment of the nucleic probes according to the invention, they may be used as capture probes. In this case, a probe, called “capture probe”, is immobilized on a support and serves to capture, through specific hybridization, the target nucleic acid obtained from the biological sample to be tested and the target nucleic acid is then detected by means of a second probe, called “detection probe”, labelled with an easily detectable element.

The nucleotide sequences according to the invention may moreover be of interest when they are used as antisense nucleotides, that is to say whose structure brings about, through hybridization with the target sequence, inhibition of the expression of the corresponding product. They may also be used as sense nucleotides which, through interaction with proteins involved in the regulation of the expression of the corresponding product, will induce either an inhibition, or an activation of this expression.

The subject of the invention is also the use of a nucleotide sequence according to the present invention for the production or the synthesis of a recombinant polypeptide.

The method of producing a polypeptide of the invention in recombinant form, which is itself included in the present invention, is characterized in that the transformed cells, in particular the cells or mammals of the present invention, are cultured under conditions allowing the expression of a recombinant polypeptide encoded by a nucleotide sequence according to the invention, and that the said recombinant polypeptide is recovered.

The recombinant polypeptides, characterized in that they can be obtained by the said method of production, also form part of the invention.

The recombinant polypeptides obtained as indicated above may be provided either in glycosylated or nonglycosylated form and may or may not have the natural tertiary structure.

The sequences of the recombinant polypeptides may also be modified in order to improve their solubility, in particular in aqueous solvents.

Such modifications are known to persons skilled in the art, such as for example the deletion of hydrophobic domains or the replacement of hydrophobic amino acids with hydrophilic amino acids.

These polypeptides may be produced from the nucleic acid sequences defined above, according to techniques for producing recombinant polypeptides known to persons skilled in the art. In this case, the nucleic acid sequence used is placed under the control of signals allowing its expression in a cellular host.

An effective system for producing a recombinant polypeptide requires having a vector and a host cell according to the invention.

These cells may be obtained by introducing into host cells a nucleotide sequence inserted into a vector as defined above, and then culturing the said cells under conditions allowing the replication and/or the expression of the transfected nucleotide sequence.

The methods used for purifying a recombinant polypeptide are known to persons skilled in the art. The recombinant polypeptide may be purified from cell lysates and extracts, from the supernatant of the culture medium, by methods used individually or in combination, such as fractionation, chromatography methods, immunoaffinity methods using specific monoclonal or polyclonal antibodies, and the like.

The polypeptides according to the present invention may also be obtained by chemical synthesis using one of the numerous known peptide syntheses, for example the techniques using solid phases (21) or techniques using partial solid phases, by condensation of fragments or by conventional synthesis in solution.

The polypeptides obtained by chemical synthesis and which may contain corresponding unnatural amino acids are also included in the invention.

The subject of the present invention is also a DNA chip, characterized in that it contains at least one nucleotide sequence in accordance with the present invention.

Actually, the nucleotide sequences according to the invention which it is envisaged to use as probe or as primer for the detection, identification, assay and/or amplification of nucleic acid sequences may be covalently or noncovalently immobilized on a support, this support being a DNA chip or a high density filter.

The expression “DNA chip” or “high density filter” is understood to mean a support to which DNA sequences are attached, it being possible for each of them to be located by its geographic localization. These chips or filters differ mainly in their size, the support material, and possibly the number of sequences attached thereto.

In particular, it is possible to carry out a synthesis in situ by photochemical targeting or by ink jet targeting. Other techniques consist in carrying out a synthesis ex situ or in attaching the probes to the support of the DNA chip by ink jet, electronic or mechanical addressing. These different methods are well known to persons skilled in the art.

The subject of the invention is also a protein chip comprising a polypeptide or antibody according to the invention.

Such a protein chip allows the study of interactions between the polypeptides according to the invention and other proteins or chemical compounds and can thus be useful for screening compounds which interact with the polypeptides according to the invention.

It may also be possible to use the protein chips according to the invention for detecting the presence of antibodies directed against the polypeptides according to the invention in the serum of patients to be tested. It may also be possible to use a protein chip comprising an antibody according to the invention, this time to detect the presence of polypeptides in the serum of patients which are capable of being recognized by the said antibodies.

The subject of the present invention is also the use of a compound chosen from a nucleotide sequence, a polypeptide, a vector, a cell or an antibody according to the invention for the preparation of a medicament.

The pathologies more specifically targeted are viral diseases and diseases characterized by the development of tumour cells or cellular degeneration such as Alzheimer's disease or schizophrenia. Thus, the abovementioned medicament is intended for the prevention and/or the treatment of these diseases. In particular, the targeted disease is cancer.

One of the benefits of the present invention is that it has demonstrated the involvement of a large number of nucleotide sequences in the phenomena of tumour suppression, tumour reversion, apoptosis and/or viral resistance. These sequences are therefore expressed differentially when one of these abovementioned processes is triggered. Consequently, in the presence of a patient in whom the onset of one of these processes is suspected or for whom it is desired to check the absence of such an onset, it is useful to be able to determine, or even quantify, the expression of one or more sequences in accordance with the invention from a biological sample from the said patient. Optionally, analysis of the expression of one or more of the said sequences may be accompanied by a comparison with a reference level of expression corresponding to that of a healthy individual.

Consequently, the invention therefore also comprises a method for the diagnosis and/or prognostic evaluation of a viral disease or a disease characterized by tumour development or cell degeneration comprising the analysis of the expression of at least one sequence of the invention from a biological sample from a patient to be tested.

According to a preferred embodiment, the said method comprises the following steps:
isolation of the messenger RNA from a biological sample obtained from a patient to be tested,
preparation of the complementary cDNA from the said messenger RNA,
optionally, amplification of a portion of the complementary DNA corresponding to at least one sequence of the invention, and
detection of the optionally amplified complementary DNA.

In particular, analysis of the expression of the sequence may be carried out by means of a DNA chip as described above.

Among the sequences of the present invention, some exhibit the characteristic of being receptors expressed at the surface of cells and in order to understand the mechanism thereof in the context of the abovementioned processes, it is useful to search for compounds capable of interacting with this receptor, that is to say of interacting with a polypeptide in accordance with the invention, it is also necessary to predict the secreted protein. This also applies to the polypeptides in accordance with the invention corresponding to secreted proteins (at the surface or outside the cells), hormone-like proteins, and the like. Consequently, the subject of the present invention is also a method for screening compounds capable of binding to a peptide in accordance with the invention and comprising the following steps:
bringing a polypeptide or a cell according to the invention into contact with a candidate compound, and
detection of the formation of a complex between the said candidate compound and the said polypeptide or the said cell.

A method for screening compounds may also be useful in relation to compounds capable of interacting with a nucleotide sequence according to the invention, or even with the sequences necessary for the expression or the regulation of these sequences. Indeed, the compounds are capable of interacting with the said sequences, the effect being to reduce, inhibit or on the contrary potentiate the expression of the sequences in question. Such a method comprises the following steps:
bringing a nucleotide sequence or a cell according to the invention into contact with a candidate compound, and
detection of the formation of a complex between the said candidate compound and the said nucleotide sequence or the said cell.

For reasons mentioned above, it may therefore be useful to search for and/or assay, in a biological specimen or sample from a patient to be tested, the presence of a nucleotide sequence according to the invention. Such a method of detection and/or assay comprises the following steps:
bringing a labelled nucleotide sequence according to the invention into contact with the biological sample to be tested, under the conditions necessary for the formation of a hybrid, and
detection and/or assay of the hybrid which may be formed between the said nucleotide sequence and the nucleic acid present in the said biological sample.

This method may additionally comprise a step for amplifying the nucleic acid of the said biological sample with the aid of primers chosen from the nucleotide sequences according to the invention.

In particular, this method may be carried out by means of the DNA chip described above.

Persons skilled in the art know how to carry out such a method and may in particular use a reagent kit comprising:
a) a nucleotide sequence according to the invention, used as a probe,
b) the reagents necessary for carrying out a hybridization reaction between the said probe and the nucleic acid of the biological sample,
c) the reagents necessary for the detection and/or assay of the hybrid formed between the said probe and the nucleic acid of the biological sample.

Such a kit may also contain positive or negative controls in order to ensure the quality of the results obtained.

Likewise, the detection and/or the assay of a polypeptide according to the invention can also be envisaged in the context of the present invention and this method therefore comprises the following steps:
bringing the biological sample into contact with a labelled antibody according to the invention, and
detection and/or assay of the complex formed by the said antibody of polypeptide present in the said sample.

Advantageously, this method may be carried out by means of the protein chip as described above. Here again, persons skilled in the art know how to carry out such a method and may in particular use a reagent kit comprising:
a) a monoclonal or polyclonal antibody according to the invention;
b) optionally reagents for preparing a medium favourable for the antigen/antibody reaction;
c) the reagents allowing the detection of the antigen/antibody complex.

Finally, the subject of the present invention is also a computer readable support or a computer support on which are recorded at least one nucleotide sequence according to the Claim 1 and/or at least one polypeptide sequence as defined in Claim 3 or 4. In particular, this support is chosen from the group comprising:
a) a floppy disk,
b) a hard disk,
c) a random access memory (RAM),
d) a read only memory (ROM),
e) a CD ROM.

The invention is not limited to the present description but encompasses on the contrary all the variants and will be understood more clearly in the light of the experimental data below.

### FIGURES

FIG. 1 represents a curve for tumour growth for the U937 and US4 groups in SCID mice.

FIG. 2 represents a curve for the body weight of mice carrying a U937 or US4 tumour.

FIG. 3 illustrates:

a/ the analysis of the expression of the TPT1/TCTP gene in the U937/US4.2 lines and in the MCF7/MCF7+SIAH1 lines by Northern blotting. The quantities of total transcripts are checked by monitoring GAPDH;

b/ Western blot analysis of the expression of the TCTP protein in the U937/US4.2 model, in the MCF7/MCF7+siah model and analysis of the expression of the murine homologue of TCTP, TCTP_MOUSE, in the M1/LTR6 system after 20 h of induction at 32° C.;

c/ the analysis of the expression of TCTP in U937 cells transfected with the antisense TPT1/TCTP. The clones I and III are compared with the control, U937 cells with the vector alone.

On the right hand panel, detection of the cleavage of PARP, a marker for apoptosis, in the clones I and III, U937 cells transfected with the antisense TPT1/TCTP. Jurkat cells treated with an anti-Fas (anti-CD95) antibody, an inducer of apoptosis, serve as a control;

d/ characterization of the apoptotic cells by double labelling FITC-Annexin V and propidium iodide (PI).

The U937 cells+control vector and the U937 cells+antisense TPT1/TCTP are simultaneously labelled with annexin V coupled to FITC (which binds to the phosphatidylserine of the apoptotic cells in the early phase) and PI (which only labels necrotic cells whose plasma membrane is damaged). Cytofluorometric analysis makes it possible to determine the percentage of apoptotic cells (Annexin+, PI−), necrotic cells (Annexin+, PI+ or Annexin−, PI+) and intact cells (Annexin−, PI−);

e/ evaluation of apoptosis in the cells transfected with the antisense TPT1/TCTP, by in situ labelling of the free 3′ ends of the nuclear DNA: TUNEL (“Terminal deoxyribonucleotidyl transferase-mediated dUTP nick end labelling”) method. This labelling makes it possible to demonstrate in situ the fragmentation of nuclear DNA resulting from the activation of endonucleases during apoptosis. The apoptotic cells appear in green.

f/ the study of the in vivo tumorigenicity of the U937 cells transfected with: the vector alone (top curve—91 tumours/100), an antisense presenilin 1 (second curve from the top—12 tumours/20), SIAH1 (middle curve—12 tumours/20) and an antisense TPT1/TCTP, clone I and III (the bottom 2 curves—8 tumours/20 and 4 tumours/20). 10 million cells injected into each flank and into each shoulder.

FIG. 4 represents cells cultured on an extra-cellular tumour matrix (Matrigel). The 184B5 line, mammary epithelial cells transformed with benzo(a)pyrene, is used as a control for the formation of acini. The tumour cells T47D and MCF7 form irregular colonies. The MCF7 cells transfected with SIAH1 partially recover an organized structure. The MCF7 and T47D cells transfected with the antisense TPT1/TCTP result in a cellular reorganization comparable to the normal formation of the acini. The cells which appear green (light spots in the figure) are labelled with an anti-E_Cadherin antibody coupled to FITC and the nuclei labelled with PI appear red (darker spots).

Western blot analysis of the expression of TCTP in the MCF7 and T47D cells transfected with the antisense TPT1/TCTP.

### 1—DATA RELATING TO THE U937 and US4 CELLS

As seen above, the present invention uses the parental cells U937 and the “derived” cells US4. Indeed, the US4 cells and the US3 cells (which are not involved in the context of the present invention) share certain characteristics. Their mode of production and their properties are presented below.

Selection and Characterization of US3 and US4 Cells

The U937 cells were subjected to two series of limiting dilution until a single clonal population is obtained. These cells were infected with the H-1 parvovirus. The cytopathic effect of the virus creates massive cell death which spares two resistant clones which are US3 and US4 after three months of continuous culture. The survival of the cells is defined as the relative number of viable cells in the culture infected with the H-1 virus compared with the untreated culture, as measured four days after reinfection. To measure the tumorigenicity, 10⁷ U937, US3 and US4 cells were subcutaneously injected into scid/scid mice (4 or 5 weeks old). The tumorigenicity is expressed by the number of tumours developed by the mice within two months following the injection.

The approach was as follows: using clonal populations of malignant cells, subclones were derived with a suppressed tumorigenic phenotype. This selection made by means of the H-1 parvovirus is carried out by the elimination of the tumour cells which are preferentially killed while sparing the normal cells. The selection of the cells resistant to the cytopathic effect of the H-1 parvovirus outside a sensitive tumour can give rise to cells which have a reduced malignant phenotype.

On this basis, a clonal population of U937 cells was isolated, these cells are sensitive to the cytopathic effect of H-1 parvovirus and the US3 and US4 clones are, for their part, resistant to the virus. The US3 and US4 clones have a strong suppressed tumorigenic phenotype while the parental U937 cells develop tumours in 80% of cases among the scid/scid mice which were infected with the parvovirus, the US3 cells form only a single tumour and the US4 cells develop one tumour per 20 inoculations with 10⁷ cells. The results are assembled in the table below.

### Resistance to the H-1 Parvovirus and Tumorigenicity of the U397, US3 and US4 Cells

| | Survival of the | Tumorigenicity |
|---|---|---|
| | cells to | in |
| | infection with | the scid/scid |
| Cell lines | the H-1 virus | mice |
| U937 | 0.4 | 16/20 |
| US3 | 96 | 0/10 |
| US4 | 89 | 1/20 |

### 2—MATERIALS AND METHODS

The aim is to compare the growth of subcutaneous tumours in SCID mice induced by subcutaneous injection of transfected U937 and US4 human leukaemic cell lines.

A. Leukaemic Cell Lines and Culture Conditions

All the injected cells of the U937 (ATCC) and US4 cell lines were provided in the form of cell suspensions in bottles supplemented with RPMI-1640 culture medium supplemented with 2 mM L-glutamine, 10% foetal calf serum and gentamycin.

The U937 cell line is a CD4+ human monocytic cell line derived from a patient having a diffuse histiocytic lymphoma (1).

The cells were counted in a haemocytometer and their viability was tested with exclusions of trypan blue dyes at 0.25%. The viability was 95.5% and 90.5% for the U937 cells and the US4 cells, respectively. The U937 and US4 cells were centrifuged and then resuspended in an RPMI medium before being injected into SCID mice.

B. The animals

10 female SCID mice in good health (CB17/IcrHsd), 31 weeks old and weighing between 20 and 25 g, were supplied by Harlan France (Gannat, France). The animals were observed for 7 days in a specific room belonging to the Applicant which is the unit for treating specific-pathogen-free animals before their treatment. The unit for treating animals (INRA, Dijon, France) is authorized by the French Ministers of Agriculture and Research (authorization No. A21100). The animal experiments are carried out according to the European Ethical Directives for the well-being of animals in the context of experimental neoplasia (3).

B.1. Environment

The animals were kept in rooms under controlled conditions of temperature (24±1° C.), humidity (55±1%), light period (12 h of light/12 h of darkness) and air renewal. The animals were kept under SPF conditions and the temperature and the humidity of the room were continuously monitored. The aeration system was programmed to give rise to 14 air renewals per hour without recirculation. Fresh air from outside passes inside a series of filters before being uniformly diffused into each room. A high pressure (2 mm) was maintained in the experimentation rooms in order to prevent contamination or diffusion of pathogens within a mouse colony. All the staff working under SPF conditions followed specific directives as regards hygiene and clothing when they were in the breeding area.

B.2. Breeding

The animals were housed in polycarbonate cages (UAR, Epinay sur Orge, France) which were equipped so as to provide them with food and water. The standard size of the cages used is 637 cm² for 10 mice according to the standard internal operating procedures. The litter for animals and made up of sterile wood shavings (UAR) and is replaced twice per week.

B.3. Food and Drink

The animal food was bought from Extralabo (Provins, France). The food was provided ad libitum and was placed on the metal lid at the top of the cage. Water was also provided ad libitum from bottles of water equipped with rubber taps. The water bottles were washed, sterilized and replaced once per week. The water supply was sterilized by filtration with a 0.2 μm absolute filter.

B.4. Identification of the Animal and the Cage

After random distribution, the animals were identified with 2 different numbers engraved on the two ears. Each cage was marked with a specific code.

C—Experimental Data and Treatments

C.1. Induction of Tumours in SCID Mice

Before cell injection, the SCID mice were randomly divided into two groups, in an amount of 5 mice per group. 10⁷ US4 or U937 tumour cells in 0.2 ml of RPMI medium were subcutaneously inoculated at time 0 for each site of injection into SCID mice. Each animal received 4 injections of tumour cells located in different areas, one in each flank and one in each shoulder.

C.2. Collection of the Tumours

When the tumours reached a volume of 1 500 mm³, the mice were killed and the tumours were collected, weighed and frozen in liquid nitrogen, stored at −80° C. and then specifically labelled.

C.3. Monitoring of the Mice

Isoflurane forene (Minerve, Bondouble, France) was used to anaesthetize the animals before injection of cells for sacrificing. After injection of tumour cells, the mice were observed for 5 hours. The viability, behaviour, body weight of the mice and the growth of the subcutaneous tumour were recorded twice per week.

During the period of experimentation, the animals were killed under anaesthetic with isoflurane by cervical dislocation if one of the following signs appears:
sign of suffering (cachexia, weakness, difficulty in moving or eating),
tumour growth up to 10% of the body weight,
tumour ulceration and persistently becoming nude,
position of the tumour interfering with movement and/or feeding,
loss of weight of 20% for three consecutive days.

An autopsy was performed on each animal to detect the possible presence of metastases or morphological abnormalities.

D—Presentation of the Data

D.1. Monitoring Parameters

The calculation for the median and mean survival time was expressed as follows:
Mean survival time=S₁/(S₂−NT)
With:
S1=sum of the daily survivors from day 0 up to the end of the experiment (without the survivors “not taken into account”*)
S2=number of animals at the beginning
NT=number of animals “not taken into consideration”*
   *“not taken into consideration”: they are animals with tumours which are smaller than the predetermined limit considered as resulting from a defect in implantation of the tumour.

D.2. System for Inhibition of the Tumour

The tumour size was measured twice per week with a compass (and the tumour volume in mm³) will be estimated according to the formula: (length×width²/2 (4). The experiments were stopped when the tumour sizes in the mice reached 1 500 mm³.
After sacrificing, the tumours were excised and weighed.
The curve for tumour growth for the US4 and U937 groups was noted using the mean of the tumour volumes.
The tumour doubling time for the US4 and U937 groups was defined as the period required to reach a mean tumour volume of 200% during the growth period.
The specific growth period over one or two doubling times (DT) from injections of cells is defined as follows:
   specific growth period=(DT US4−DT U937)/DT U937.
The growth period is calculated as the difference between the median growth time for the US4 group and the U937 group in order to reach the same tumour size.

D.3. Statistical Tests

All the statistical analyses were carried out with the StatView® software (Abacus Concept, Berkeley, USA). The statistical analyses of the changes in mean body weight, the tumour doubling time and the time to reach “V” were performed using the Bonferroni/Dunn test. A value p<0.05 was considered to be significant. All the groups were compared with each other.

E—Results

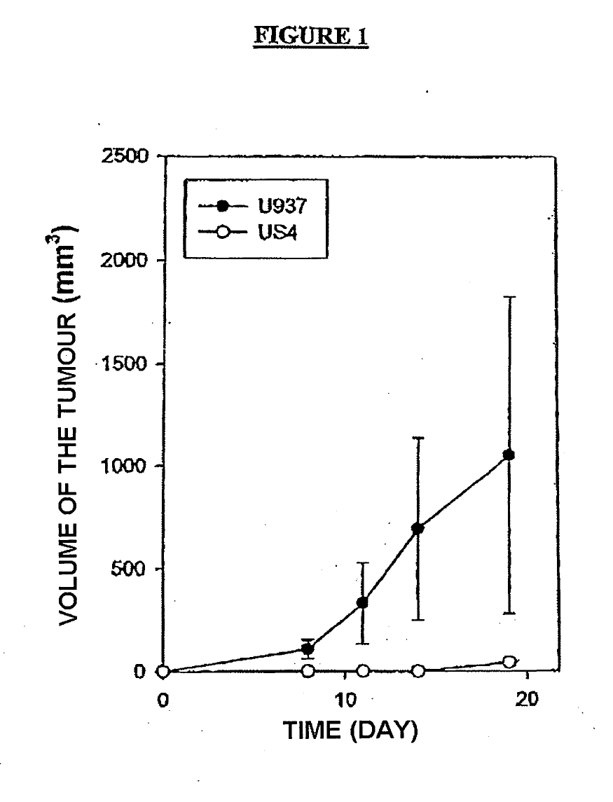

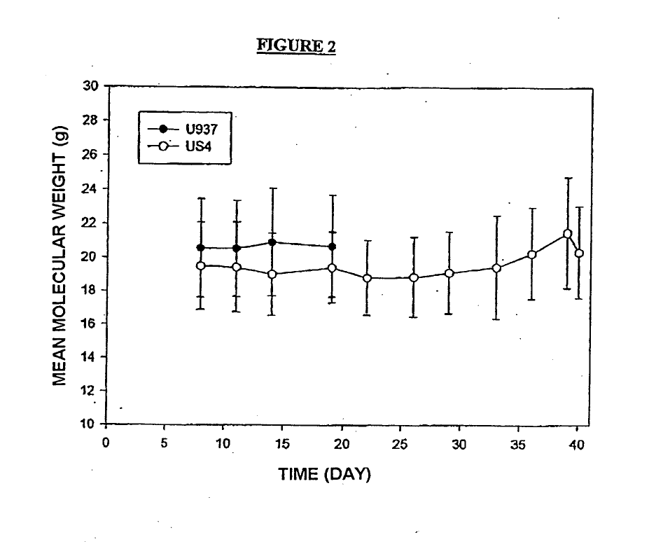

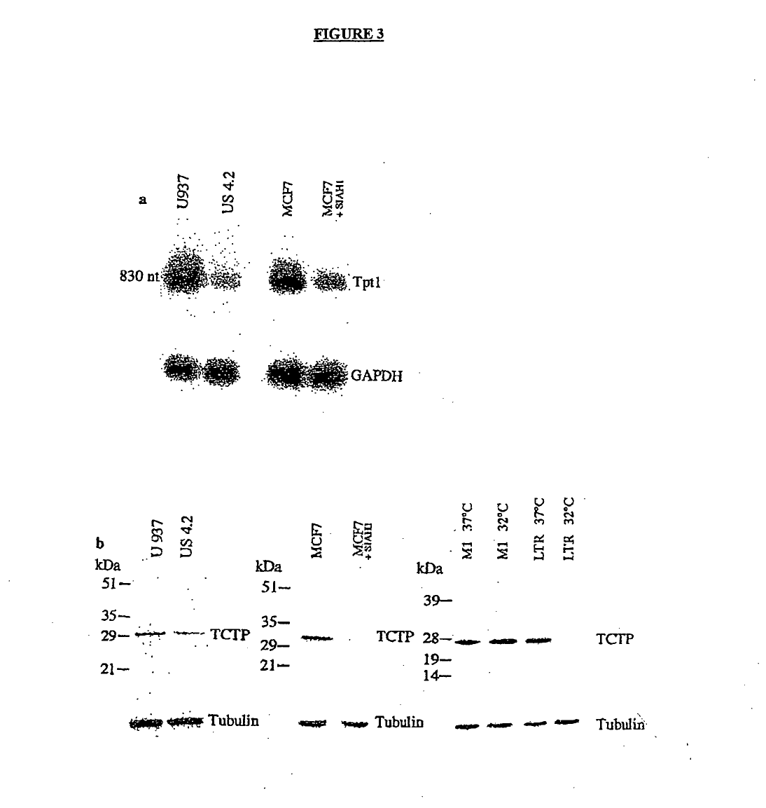

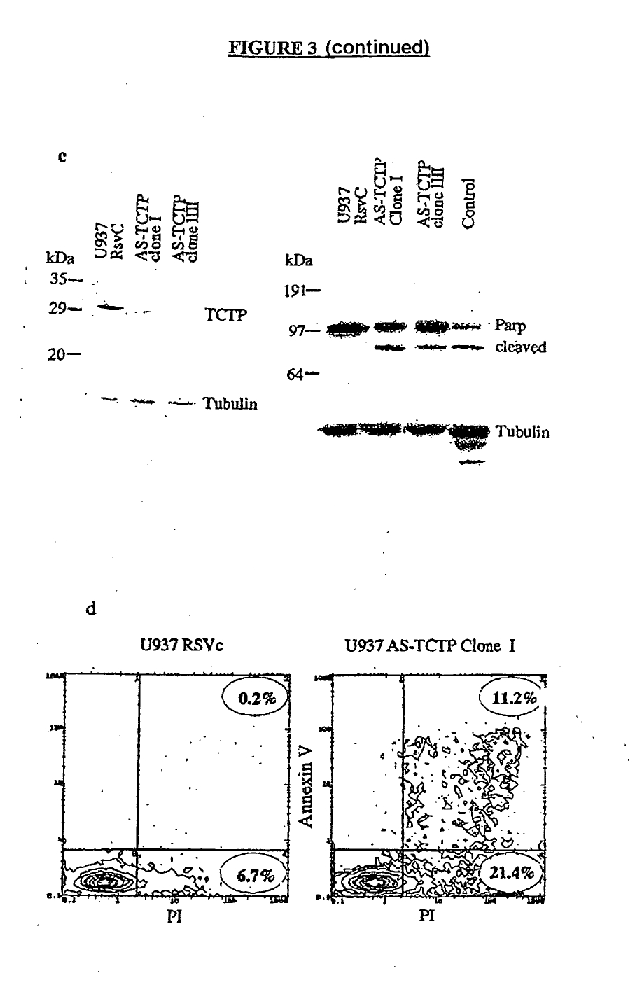

The curves for mean tumour volumes and body weights are shown in FIGS. 1 and 2 respectively.

No significant loss of body weight in SCID mice from the two groups was observed between day 8 and day 19.

A significant difference is observed as regards the time to reach “V” between the 2 groups of SCID mice whereas no significant difference was observed for the change in mean body weight (day 19−day 8) and for the doubling time.

The autopsies performed did not show the presence of metastases or of developments of suspicious nodosity. The tumours were collected on animals sacrificed after anaesthetic and cervical dislocation. The tumours were immediately placed in tubes, frozen in liquid nitrogen and stored at −80° C. The excised tumours had an ovoid shape, a moderate consistency and a pinkish colour. The interactions of the tumour with its environment (skin and muscle tissue) were limited and superficial.

F—Conclusions

The US4 cell line showed an uptake level significantly lower compared with the U937 cell line in the SCID mice.

The retardation of growth between the US4 and U937 tumours was 23.5 days and the doubling time was equivalent.

### 3—INVOLVEMENT OF TPT1/TCPT IN TUMOUR REVERSION

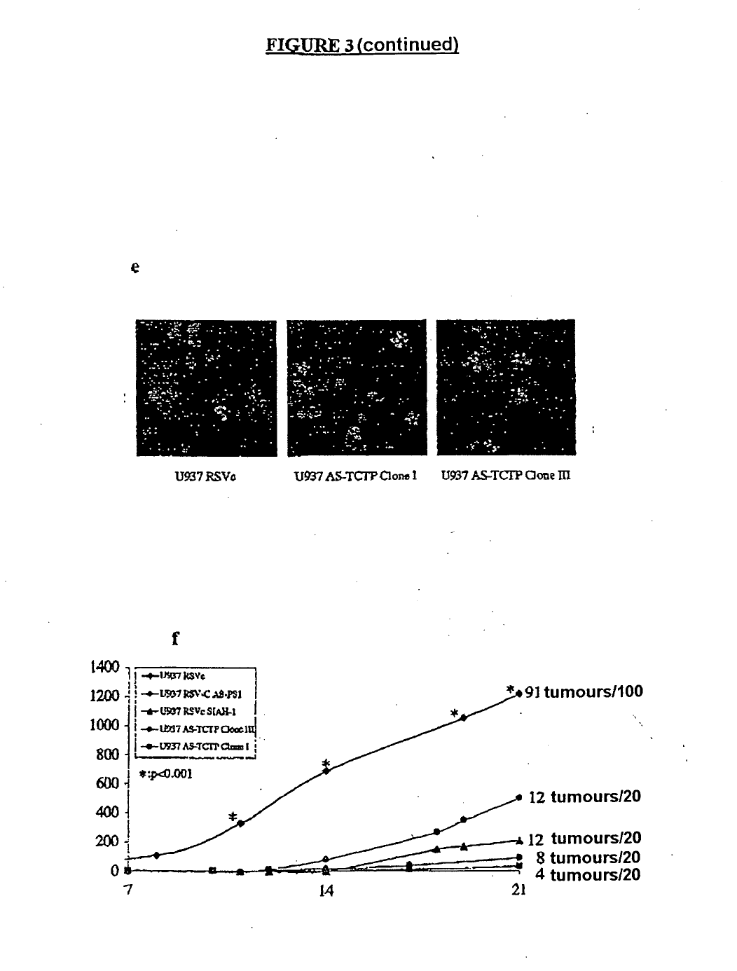

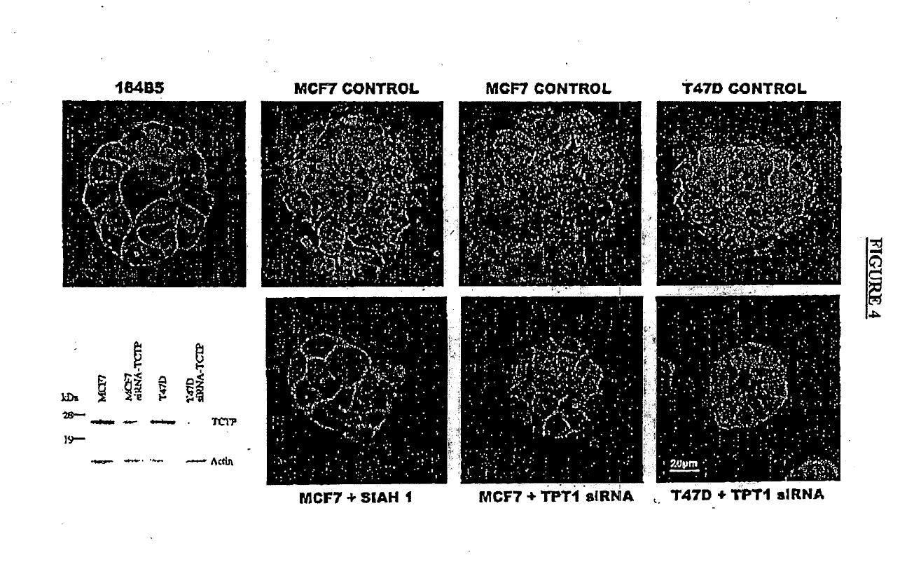

In order to study the involvement of TPT1/TCPT (Translationally controlled Tumour Protein encoding the Histamine Release Factor) in tumour reversion, the expression of the gene (FIG. 3a) and the expression of the protein (FIG. 3b) in various cellular models of tumour reversion: U937/US4.2, MCF7/MCF7+SIAH1 and M1/LTR6, were analysed. The level of expression of TPT1/TCTP is highly reduced in the revertant cells compared with the tumour cells.

To deal with the physiological aspect of this reduction, the U937 tumour cells were transfected with the antisense TPT1/TCTP. After having checked the drop in the expression of TPT1/TCTP (FIG. 3c, left panel), the inventors detected its consequences in apoptosis by three different methods: detection of the cleavage of PARP, labelling with Annexin V and TUNEL method (FIG. 3c, right panel; FIG. 3d; FIG. 3e, respectively).

The reduction in the expression of TPT1/TCTP in tumour cells in culture increases apoptosis.

The inventors then expressed in vivo the physiological consequences of this loss of TPT1/TCTP functions. We injected U937 cells transfected with antisense TPT1/TCTP into mice and observed a drastic reduction in the number of tumours formed when the expression of TPT1/TCTP is reduced (FIG. 3f).

In parallel with these results, the reduction of TPT1/TCTP, obtained by transfection of double-stranded RNA specific for TPT1/TCTP (siRNA), into MCF7 and T47D cells, cultured on an extracellular matrix, leads to a cellular reorganization of acini which is comparable to a normal structure (FIG. 4).

### REFERENCES

(1) Sundström C. and Nilsson K., Int. J. Cancer, 17, 565, 1976.
(2) Principe d'éthique de l'expérimentation animale [Principle of ethics of animal experimentation], Directive No. 86/609 EEC of 24 Nov. 1986, Decree No. 87/848 of 19 Oct. 1987, Application Order of 19 Apr. 1988.
(3) United Kingdom co-ordinating committee on cancer research guidelines for welfare of animals in experimental neoplasia, Br. J. Cancer, 77: 1-10, 1998.
(4) Bissery M. C. et al., Bull. Cancer, 78: 587, 1991.
(5) Perricaudet et al. (1991). La Recherche 23: 471.
(5a) Epstein (1992), Médecine/Sciences, 8, 902.
(6) Temin (1986), Retrovirus vectors for gene transfer. In Kucherlapati R., ed. Gene.
(7) Carter (1993), Curr. Op. Biotechnology 3, 533.
(8) Olins and Lee (1993), Curr. Op. Biotechnology 4: 520.
(9) Buckholz (1993), Curr. Op. Biotechnology 4, 538.
(10) Edwards and Aruffo (1993), Curr. Op. Biotechnology 4, 558.
(11) Luckow (1993), Curr. Op. Biotechnology 4, 564.
(11a) Rolfs, A. et al. (1991), Berlin: Springer-Verlag.
(12) Walker (1992), Nucleic Acids Res. 20: 1691.
(13) Kwoh et al. (1989), Proc. Natl. Acad. Sci. USA, 86, 1173.
(14) Guatelli et al. (1990), Proc. Natl. Acad. Sci. USA, 87, 1874.
(15) Kievitis et al. (1991), J. Virol. Methods, 35, 273.
(16) Landegren et al. (1988) Science 241, 1077.
(17) Segev (1992), Kessler C. Springer Verlag, Berlin, New York, 197-205.
(18) Duck et al. (1990), Biotechniques, 9, 142.
(19) Miele et al. (1983), J. Mol. Biol., 171, 281.
(20) Matthews et al. (1988), Anal. Biochem., 169, 1-25.
(21) Stewart and Yound (1984), Solid phase peptides synthesis, Pierce Chem. Company, Rockford, 111, 2nd ed. (1984).

1. An isolated nucleotide sequence comprising a nucleotide sequence chosen from the group comprising:
a) SEQ. ID NO. 72,
b) a nucleotide sequence of at least 15 consecutive nucleotides of a sequence as defined in a),
c) a nucleotide sequence having a percentage identity of al least 80%, after optimal alignment, with a sequence defined in a) or b),
d) a nucleotide sequence which hybridizes under high stringency conditions with a sequence defined in a) or b), and
e) a complementary nucleotide sequence or the RNA sequence corresponding to a sequence as defined in a), b), c) or d).

2-30. (canceled)


SEQUENCE LISTING (1020 sequences)

SEQ ID NO: 1 - DNA, 178 bp, HOMO SAPIENS
gytctggaagctttttattgttgctccaagtttaagaattttcactgatggcagaaaaccaacacttttagacaatctcccgcatagccctgcccagcttctgaatcttggtcttgacagacatgtcaacatatttctcgccaatgcgcacaatcattccacccaagattgacggatc

SEQ ID NO: 2 - DNA, 182 bp, HOMO SAPIENS
tttgggttttttcttaaacctcccttaaagattctttgatgctttgctctatcactgtagacctggtctttttccccccagctttttcttttttacattctgggttgctattttcagattaataatttgatgaccccatcacagtaccaaaataccccccaaaatgaagttcaaatttgatc

SEQ ID NO: 3 - DNA, 172 bp, HOMO SAPIENS
gatccggaaatatggcctcaatatgtgccgccagtgtttccgtcagtacgcgaaggatatcggtttcattaagttggactaaatgctcttccttcagaggattatccggggcatctactcaatgaaaaaccatgataattctttgtatataaaataaacatttgaaaaaacc

SEQ ID NO: 4 - DNA, 168 bp, HOMO SAPIENS
gatcggtgacatcgtcacagtgggcgagtgccggcctctgagcaagacagtgcgcttcaacgtgctcaaggtcaccaaggctgccggcaccaagaagcagttccagaagttctgaggctggacatcggcccgctccccacaatgaaataaagttattttctcattccc

SEQ ID NO: 5 - DNA, 167 bp, HOMO SAPIENS
caaaccctgttcttgggtgggtgtgggtataatactaagttgagatgatatcatttacgggggaaggcgctttgtgaagtaggccttatttctcttgtcctttcgtacagggaggaatttgaagtagatagaaaccgacctggattactccggtctgaactcagatc

SEQ ID NO: 6 - DNA, 163 bp, HOMO SAPIENS
gatctcaggcatcggattatttcttctgtaaatatttcagaatgtatctctccaagatgagagctcattaaaagataattacaaagcttatcacatccaaaagaattatcaataattttgaaatattattaaacgtgtaataaatgttcaaagttccacttgc

SEQ ID NO: 7 - DNA, 161 bp, HOMO SAPIENS
gatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgtttaaataaatgtaaaaactgcc

SEQ ID NO: 8 - DNA, 160 bp, HOMO SAPIENS
gatcaatgaagtgagaaattgttcagaaggatacagtttgtttttagatgtcctttgtccaatgtgaacatttattcatattgttttgattaccctcgtgttactacaagatggcaataaatactatgggattgtttgtattaaaaaaaaaaaaaaataa

SEQ ID NO: 9 - DNA, 308 bp, HOMO SAPIENS
gatctctggcagtggaggaagtctctttaagaaaatagtttaarcaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaa

SEQ ID NO: 10 - DNA, 159 bp, HOMO SAPIENS
gatcacctctgagacccaccttgctcataaacaaaatgcccatgttggtcctctgccctggacctgtgacattctggactatttctgtgtttatttgtggccgagtgtaacaaccatataataaatcacctcttccgctgttttagctgaagaattaaa

SEQ ID NO: 11 - DNA, 151 bp, HOMO SAPIENS
attcggytcagtctaatcctttttgtagtcactcataggccagacttagggctaggatgatgattaataagagggatgacataactattagtggcaggttagttgtttgtagggctcatggtaggggtaaaaggagggcaatttctagatc

SEQ ID NO: 12 - DNA, 149 bp, HOMO SAPIENS
gatcctactcctttggagtaaaactagtgcttaccagtttccaattgtatttagcttctggttggaatttgaaaaaaaaaagaaaaaaagaaaaagaaaacctaaataaaataggtgaaagttccctgactattcaggtgaatacaccc

SEQ ID NO: 13 - DNA, 148 bp, HOMO SAPIENS
gatccaagaggaagaatccagctgcctatgaaaatgacaaatgagcaacgcatccggatgacggttccctgtctctgaaagacctttctctggaagaggagtctgcattgtagtgtctcaaagacacaataaacttcctatggtctgc

SEQ ID NO: 14 - DNA, 146 bp, HOMO SAPIENS
gatctgacccaccagtttgtacatcacgtcctgcaygtcccacaccattttttcatgaccttgtartatactggtctctgtgctatagtggaatctttggttttgcatcatagtaaaataaaataaacccatcacatttggaacac

SEQ ID NO: 15 - DNA, 145 bp, HOMO SAPIENS
gatctggctgaaccagttccacaaggttactgtatacatagcctgagtttaaaaggctgtgcccacttcaagaatgtcattgttagactttgaaatttctaactgcctacctgcataaagaaaataaaatcttttaaatcaaaaa

SEQ ID NO: 16 - DNA, 140 bp, HOMO SAPIENS
gatctggtctagttaacctagaagtatttttgtctcttagaaatacttgtgatttttataatacaaaagggtcttgactctaaatgcagttttaagaattgtttttgaatttaaataaagttacttgaatttcaaacatc

SEQ ID NO: 17 - DNA, 139 bp, HOMO SAPIENS
tttttgctgtaggcccgggtggttgctgccraaatgggcaagttcatgaaacctgggaaggtggtgcttgtcctggctggacgctactccggacgcaaagctgtcatcgtgaagaacattgatgatggcacctcagatc

SEQ ID NO: 18 - DNA, 138 bp, HOMO SAPIENS
actgaatgacgtttaatgaacaaatgaggaattggttatttatactctgtggacgtttattaaggctctggcaacactgtggagctggcctgtgtccccgagaagaagagtcagtcacaggggggtctccatctgatc

SEQ ID NO: 19 - DNA, 231 bp, HOMO SAPIENS
gtgaatgtaaaacatttaatttaaaaatgttgatactacaatatataaaatagctattataaatgcacatagtgtattctatagctgccaggtttacttttttttttttttaaaggaaactgtaagttacactgtggttaagacttgyatcttcacccttgaaaaagcccacattctatcacagtgatgtatggtcagacttaacagccccaattgttaaacacttggatc

SEQ ID NO: 20 - DNA, 138 bp, HOMO SAPIENS
gatccaggtgactctgacatcattagaagcatgccagaacagactggtgaaaagtaaaccttttcacctacaaaatttcacctgcaaaccttaaacctgcaaaattttcctttaataaaatttgcttgttttaaaaac

SEQ ID NO: 21 - DNA, 138 bp, HOMO SAPIENS
gagttaaatacaggcttttttttttaatctgtaattgatttacatttgattttaagaagacactgtttacatcaggtcaccacaaagcagatttacatacaaccttcaattgcggaaaaacaaagtttctttttgatc

SEQ ID NO: 22 - DNA, 137 bp, HOMO SAPIENS
gatccttttatcctgctacagtctttagggagctaaaaaaaaaaaaaaagcaggggctgccaaaactcttgatttcatatttccttctctaaatatatatgtatcctgttttttggataaaattttaccaagaatcc

SEQ ID NO: 23 - DNA, 133 bp, HOMO SAPIENS
gatcccaaatttgaagtcatcgaaaaaccccaggcctgaagaaataaagtaaaattaatctggtaatttgtcacggattagttgtacaactagttagaagtttcagaataaacatgcatttcataactgtcaa

SEQ ID NO: 24 - DNA, 131 bp, HOMO SAPIENS
gggttattaaataatttgtttattgtacggcatttacaaagaaaacagacaatgccctcagtagaaagaataaaaatgtatttagggctttatttttaactgacagcaaatagaaatcctttagtgagatc

SEQ ID NO: 25 - DNA, 130 bp, HOMO SAPIENS
gatcatgttaccatatcaagctgaaaatgtcaccactatctggagatttcgacgtgttttcctctctgaatctgttatgaacacgttggttggctggattcagtaataaatatgtaaggcctttcttttt

SEQ ID NO: 26 - DNA, 128 bp, HOMO SAPIENS
gatcaattacctaactgttccttggtctatttatgtataagaatgctttttaaagcacatgtctcattttaaatgacgcacaaactgaagatgttaataaaatttaagagtaatacaawraaaaacaa

SEQ ID NO: 27 - DNA, 126 bp, HOMO SAPIENS
gatcctcgaacggaaagccaaatctcgccaagtaggaaaggaaaagggcaaatacaaggaagaaaccattgagaagatgcaggaataaagtaatcttatatacaagctttgattaaaacttgaaac

SEQ ID NO: 28 - DNA, 125 bp, HOMO SAPIENS
gcaggttagcattttattagataaaaacaggaaggagggtgcctacccacccctgcctrctactggctctgaagggcactcctcaacttccccaagaaagaggacgcgyctctgacactgtgatc

SEQ ID NO: 29 - DNA, 239 bp, HOMO SAPIENS
gatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaaagaaaa

SEQ ID NO: 30 - DNA, 119 bp, HOMO SAPIENS
gatctaaataagtgtttcaaggaaagttttcytaagcaaatgtaatattacctcatttgggcatcattactctgttaattctatatcaaaggaaataaacttgctacttgcactaaatg

SEQ ID NO: 31 - DNA, 117 bp, HOMO SAPIENS
gatcgggtagctcaaaagaaggcaagcttcctcagagctcaggagcgggctgctgagagctaaacccagcaattttctatgattttttcagatatagataataaacttatgaacagc

SEQ ID NO: 32 - DNA, 117 bp, HOMO SAPIENS
gatctggacagcagaatgttataacgcaagttcatgtgttgctcccarctccattctcttwtctctcgtgcaaccagtttgcccattctcttcctattacttgctccagggataggt

SEQ ID NO: 33 - DNA, 117 bp, HOMO SAPIENS
gttaatgaaaataagctttattacatcaagtaataaatacatacaaagatgcaaacagttttagtcattttcttccagatgtttttatcaacttacaataaacgcagaactgagatc

SEQ ID NO: 34 - DNA, 116 bp, HOMO SAPIENS
gatcactgagtgtacagaagagagaaattcaaacaaaatattgctgttcttcagttttgtttgtggaatttgaaattactcaaatttaaaataaattactggactgtggaaataac

SEQ ID NO: 35 - DNA, 116 bp, HOMO SAPIENS
gatcaccttagtttgattctattttttagcttgcaaaaagtgacttatattccaaagaaattaaaatgttgaaatccaaatcctagaaataaaatgagttaacttcaaacatttca

SEQ ID NO: 36 - DNA, 110 bp, HOMO SAPIENS
gatcaaggtctgcaagggaattcttgtgtgctgctttccatttgacaccgcagttctgttcagccatcagaagagagacaaggaattaaaaatttctttttaatcctgtt

SEQ ID NO: 37 - DNA, 110 bp, HOMO SAPIENS
gatctttagcacagtgatatcaccatgacttcacagacatggtctagaatctgtacccttacccacatatgaagaataaaattgattaaaggtttttttggtgagacttt

SEQ ID NO: 38 - DNA, 106 bp, HOMO SAPIENS
cacactacaatagttaattttatttgttcaagagctcagattgcaagcattaaaccaagcataggctttgattctgtgagcccaaattcacatattgaagaagatc

SEQ ID NO: 39 - DNA, 101 bp, HOMO SAPIENS
gatctccaaagtggtgagtttatgtgtgatttttattttgtttatgctcttctgtattttccgaatttcatacaataaatatctgttacttttacaatatg

SEQ ID NO: 40 - DNA, 205 bp, HOMO SAPIENS
gatccacttctgtgattaagtaaatggatgtctcgtgatgcgtctacagttatttattgttacatccttttccagrcactgtagatgctataataaaaatagctgtttggtaaccatagtttcacttgttcaaagctgtgtaatcgtgggggggtactatctcaactgcttttgtattcattgtattaaaagaatctgtttaaac

SEQ ID NO: 41 - DNA, 98 bp, HOMO SAPIENS
gatctaagagttacctggctacagaaagaagatgccagatgacacttaagacctacttgtgatatttaaatgatgcaataaaagacctattgatttgg

SEQ ID NO: 42 - DNA, 94 bp, HOMO SAPIENS
caatttgagagcaggtactgtttattaaccaaccagcttagaaaaataatcatggtagacaccttagttcattcttctaataagcctgttgatc

SEQ ID NO: 43 - DNA, 94 bp, HOMO SAPIENS
atagttcggagctgtttattgagggcaggggaagaatgtcataaggtacattatcataaggttacagagcacagtgtcatgaggttgattgatc

SEQ ID NO: 44 - DNA, 92 bp, HOMO SAPIENS
gatctgatgttccttttatttataacaatgttaaatgcaattgtcttgtaccttgagttgagtattacacattaaagtaaagtacaagctgt

SEQ ID NO: 45 - DNA, 89 bp, HOMO SAPIENS
gatcatctaattctctgtggaatgaatacacacatatatattacaagggataatttagaccccatacaagtttataaagagtcattgty

SEQ ID NO: 46 - DNA, 89 bp, HOMO SAPIENS
gatcacccccaggaggggaaaatggtggcaatgccttttatatattatgtttttactgaaattaactgaaaaaatatgaaaccaaaagt

SEQ ID NO: 47 - DNA, 88 bp, HOMO SAPIENS
acsaatgaggcaatttattaacccagcatggtttgttctaatgcttcttgttggcagctgccacctgtccggcgattctgtccagatc

SEQ ID NO: 48 - DNA, 86 bp, HOMO SAPIENS
gatctgtgtttgctctgacgaatggaatttatcctcacaaattggtgttctaaatgtcttaagaacctaattaaatagctgactac

SEQ ID NO: 49 - DNA, 77 bp, HOMO SAPIENS
gatcttgtacccgctaaaactgcacagctcgaccttcatggcgacagctccacggaaagacaaaagatggcgaarag

SEQ ID NO: 50 - DNA, 75 bp, HOMO SAPIENS
gatctacatacaaacaatgcaaccaactatccaagtgttataccaactaaaacccccaataaaccttgaacagtg

SEQ ID NO: 51 - DNA, 205 bp, HOMO SAPIENS
gagatggagttttattcttgttgcccaggctagagtacaatggtgcagtcccagctcaacctccatctcctgagttccaagcagttcccctgtctcagcctcccgagtagctgggattacagacatgcgccaccatgcccaactaatttttttgcatttttagtagagacagggtttcaccatgttwkccaggatggtctcgatc

SEQ ID NO: 52 - DNA, 74 bp, HOMO SAPIENS
gatccaccacccagcttcacccctcacccccagcggctcaccatggggatggcagcaataaaatattttcccrc

SEQ ID NO: 53 - DNA, 74 bp, HOMO SAPIENS
cgatcttaagtcatacattttaattgtgtasaggttgtwcaactgaaggaataaatgtctattaaactaaavaa

SEQ ID NO: 54 - DNA, 72 bp, HOMO SAPIENS
gatccacgatacagtgctttggcaaagttaagtgaaaaaagcaagcctttaatgcctataaagtccagacag

SEQ ID NO: 55 - DNA, 72 bp, HOMO SAPIENS
gaaacagcacaggaaaagtgactgctttaatgtattaggcaaaattttacataaaatcagaaatctatgatc

SEQ ID NO: 56 - DNA, 69 bp, HOMO SAPIENS
gatctcattccagagaaagaggtagaaggtcaaggagcacatcaaaaacaagagacaaaaaaaacaaaa

SEQ ID NO: 57 - DNA, 68 bp, HOMO SAPIENS
gatgttaatgactttactttgagatatgatggaaaaatattacaggtacacatggaaaagacatgatc

SEQ ID NO: 58 - DNA, 66 bp, HOMO SAPIENS
gatctctttgtagccatcctgttaaatttgtaaacaatctaattaaatgrcatcagcactttaacc

SEQ ID NO: 59 - DNA, 64 bp, HOMO SAPIENS
ggaagtctaagtaggaatttatttacacggacactcaatcatagataaggcgctgtctaggatc

SEQ ID NO: 60 - DNA, 64 bp, HOMO SAPIENS
atggtgaaaagatatatatatatatatattcagaattaggcagctggactcartttagatgatc

SEQ ID NO: 61 - DNA, 200 bp, HOMO SAPIENS
gatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctc

SEQ ID NO: 62 - DNA, 62 bp, HOMO SAPIENS
gatcagaatgttctaaattttgactagttactttttattatgagttaatatagtttagcagt

SEQ ID NO: 63 - DNA, 59 bp, HOMO SAPIENS
gatccagttctaagtgtcatcttttattatgaagacaataaaatcttgagtttatgttc

SEQ ID NO: 64 - DNA, 58 bp, HOMO SAPIENS
cacgtcataagtttattgacaaacatatctagtatggcatatgagttctagtttgatc

SEQ ID NO: 65 - DNA, 58 bp, HOMO SAPIENS
aatttttctttagtttattgaaagaaatgtttacaacatgacaccaacagaagggatc

SEQ ID NO: 66 - DNA, 58 bp, HOMO SAPIENS
gatcacaccactgcactccagcctaggcaacagagactctgtctcaagaaaaacaaaa

SEQ ID NO: 67 - DNA, 53 bp, HOMO SAPIENS
gatctcctcccttgtttaaatgtcaataaatgccccaactgctttgtaagtgc

SEQ ID NO: 68 - DNA, 50 bp, HOMO SAPIENS
gagacagggtcttgttckatcaccccaggctggagtgcagtggtgtgatc

SEQ ID NO: 69 - DNA, 49 bp, HOMO SAPIENS
gatctgttcagtgtcactctgtaccctcaacatatatcccttgtgcgac

SEQ ID NO: 70 - DNA, 49 bp, HOMO SAPIENS
gaaatggagtcttgctctgtcgcccaggctggagtacaatggcgtgatc

SEQ ID NO: 71 - DNA, 43 bp, HOMO SAPIENS
gctattaatcctgccacaatatttttaattacgtacaaagatc

SEQ ID NO: 72 - DNA, 193 bp, HOMO SAPIENS
caaatgagtttaaatgcattttatttttagacaacctacatgacatgtttttcttaaaaacaatgcctccactccaaataaatcacagtcaaaataaatgaagagctcaagatgacatcagtcccatttgtcttaagtcctggtgttgtgtggatgacaagcagaagccagttatgatgacaggtgatagatc

SEQ ID NO: 73 - DNA, 42 bp, HOMO SAPIENS
gatccctgcccagcaccttcctatagagatgactttaaaagg

SEQ ID NO: 74 - DNA, 41 bp, HOMO SAPIENS
tttttgtggttttctttttaattatctttagtcttgtgatc

SEQ ID NO: 75 - DNA, 39 bp, HOMO SAPIENS
gatcatggcatttaaataaaagattgaaagattaaaaac

SEQ ID NO: 76 - DNA, 35 bp, HOMO SAPIENS
gatctcgtgaagcccgcaaggaccgaacaccccca

SEQ ID NO: 77 - DNA, 35 bp, HOMO SAPIENS
gcagattaaggcttgttttattttaatggctgatc

SEQ ID NO: 78 - DNA, 35 bp, HOMO SAPIENS
gtcagcaaaaatctttttaataagagagtaggatc

SEQ ID NO: 79 - DNA, 30 bp, HOMO SAPIENS
gatccatctgcctttgtggctgctgcccct

SEQ ID NO: 80 - DNA, 27 bp, HOMO SAPIENS
gatctgacccctattgtggtagaagac

SEQ ID NO: 81 - DNA, 20 bp, HOMO SAPIENS
cttctgaatttttttagatc

SEQ ID NO: 82 - DNA, 14 bp, HOMO SAPIENS
gatcagcgctttac

SEQ ID NO: 83 - DNA, 182 bp, HOMO SAPIENS
gatcaaagacatcctcatccagtatgaccggaccctgctggtagctgaccctcgtcgctgcgagtccaaaaagtttggaggccctggtgcccgcgctcgctaccagaaatcctaccgataagcccatcgtgactcaaaactcacttgtataataaacagtttttgagggattttaaagtttc

SEQ ID NO: 84 - DNA, 182 bp, HOMO SAPIENS
gacaattgttatttagtttttatttcataatcataaacttaactctgcaatccagctaggcatgggagggaacaaggaaaacatggaacccaaagggaactgcagcgagagcacaaagattctaggatactgcgagcaaatggggtggaggggtgctctcctgagctacagaaggaatgatc

SEQ ID NO: 85 - DNA, 188 bp, HOMO SAPIENS
gatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactc

SEQ ID NO: 86 - DNA, 156 bp, HOMO SAPIENS
gatcacctctgagacccaccttgctcataaacaaaatgcccatgttggtcctctgccctggacctgtgacattctggactatttctgtgtttatttgtggccgagtgtaacaaccatataataaatcacctcttccgctgttttagctgaagaatt

SEQ ID NO: 87 - DNA, 98 bp, HOMO SAPIENS; identical to SEQ ID NO: 41

SEQ ID NO: 88 - DNA, 132 bp, HOMO SAPIENS
gatccaggtgactctgacatcattagaagcatgccagaacagactggtgaaaagtaaaccttttcacctacaaaatttcacctgcaaaccttaaacctgcaaaattttcctttaataaaatttgcttgtttt

SEQ ID NO: 89 - DNA, 30 bp, HOMO SAPIENS; identical to SEQ ID NO: 79

SEQ ID NO: 90 - DNA, 178 bp, HOMO SAPIENS
attgttatttagtttttatttcataatcataaacttaactctgcaatccagctaggcatgggagggaacaaggaaaacatggaacccaaagggaactgcagcgagagcacaaagattctaggatactgcgagcaaatggggtggaggggtgctctcctgagctacagaaggaatgatc

SEQ ID NO: 91 - DNA, 184 bp, HOMO SAPIENS
gatcagaaaaagaaagaagccaaagagaaaggtacctgggttcaactaaagcgccagcctgctccacccagagaagcacactttgtgagaaccaatgggaaggagcctgagctgctggaacctattccctatgaattcatggcataataggtgttaaaaaaaaaataaaggacctctgggctac

SEQ ID NO: 92 - DNA, 86 bp, HOMO SAPIENS; identical to SEQ ID NO: 48

SEQ ID NO: 93 - DNA, 161 bp, HOMO SAPIENS; identical to SEQ ID NO: 7

SEQ ID NO: 94 - DNA, 265 bp, HOMO SAPIENS
gatcccagactggttcttgaacagacagaaggatgtaaaggatggaaaatacagccaggtcctagccaatggtctggacaacaagctccgtgaagacctggagcgactgaagaagattcgggcccatagagggctgcgtcacttctggggccttcgtgtccgaggccagcacaccaagaccactggccgccgtggccgcaccgtgggtgtgtccaagaagaaataagtctgtaggccttgtctgttaataaatagtttatatacc

SEQ ID NO: 95 - DNA, 180 bp, HOMO SAPIENS
gttacataaattaacccatttattataggccagtgatgtctcaaagagtagaggagcgtctactggtctttcaactccttcagtcttctgatggcggactttaccgtgacagcggaagtggtattgtacgtccaggcaccgccagccactgtcttcatgcaggaaccacagtgccagatc

SEQ ID NO: 96 - DNA, 163 bp, HOMO SAPIENS
gatcgtttccagatgagaattcacaagcgactcattgacttgcacagtccttctgagattgttaagcagattacttccatcagtattgagccaggagttgaggtggaagtcaccattgcagatgcttaagtcaactattttaataaattgatgaccagttgtt

SEQ ID NO: 97 - DNA, 130 bp, HOMO SAPIENS; identical to SEQ ID NO: 25

SEQ ID NO: 98 - DNA, 167 bp, HOMO SAPIENS; identical to SEQ ID NO: 5

SEQ ID NO: 99 - DNA, 182 bp, HOMO SAPIENS; identical to SEQ ID NO: 83

SEQ ID NO: 100 - DNA, 216 bp, HOMO SAPIENS
gactgtcctaaattgtttattaagtatgaattttacaaactttacttatattagcggtaacggtggagctggagagtattgcgccttctccaagctgcccggcgagagccaccaatagtgtggtggaacttgtggccctttccaaggccacggctctttcggcctgcagatgtcagcccacgcatctccctgtgcttgtggactggtttggtgatc

SEQ ID NO: 101 - DNA, 168 bp, HOMO SAPIENS; identical to SEQ ID NO: 4

SEQ ID NO: 102 - DNA, 43 bp, HOMO SAPIENS; identical to SEQ ID NO: 71

SEQ ID NO: 103 - DNA, 42 bp, HOMO SAPIENS; identical to SEQ ID NO: 73

SEQ ID NO: 104 - DNA, 159 bp, HOMO SAPIENS
gttgtcccagatttattgaaaataatacagcactacagaaaaaattcaaacaggtccccgaggcgttttgaaattcatcccaactgtaggctgagtgacctgaaggttggacagactgccgaagtccaaaagcttcagcatttccttagtgtcaggatc

SEQ ID NO: 105 - DNA, 149 bp, HOMO SAPIENS
rctaggcaaagaactttattaatctttgtttcaaacttgattcccaggcttcttcggcttaattagctgcaaagaatgaattgtgtataagcaaaaactgaaaagagctgcagtgtccaaggggcttgggcttaaaaatattagagatc

SEQ ID NO: 106 - DNA, 35 bp, HOMO SAPIENS; identical to SEQ ID NO: 76

SEQ ID NO: 107 - DNA, 77 bp, HOMO SAPIENS
caccctcatgttcatacacctatcccccattctcctcctatccctcaaccccgacatcattaccgggttttcctctt

SEQ ID NO: 108 - DNA, 49 bp, HOMO SAPIENS
gatctgttcagtgtcactctgtaccctcaacatatatcccttgtgcgay

SEQ ID NO: 109 - DNA, 59 bp, HOMO SAPIENS; identical to SEQ ID NO: 63

SEQ ID NO: 110 - DNA, 35 bp, HOMO SAPIENS; identical to SEQ ID NO: 77

SEQ ID NO: 111 - DNA, 151 bp, HOMO SAPIENS
attcggttcagtctaatcctttttgtagtcactcataggccagacttagggctaggatgatgattaataagagggatgacataactattagtggcaggttagttgtttgtagggctcatggtaggggtaaaaggagggcaatttctagatc

SEQ ID NO: 112 - DNA, 113 bp, HOMO SAPIENS
ctccgctactgagacacggcggacacacacaaacacagaaccacacagccagtcccaggagcccagtaatggagagccccaaaaagaagaaccagcagctgaaagtcgggatc

SEQ ID NO: 113 - DNA, 90 bp, HOMO SAPIENS
gatccaaatttataagaaagtactttaaaaatcagacaaaagacttaagaattactatgttaaatttctaatccaaatgtaaaacaaaaa

SEQ ID NO: 114 - DNA, 172 bp, HOMO SAPIENS; identical to SEQ ID NO: 3

SEQ ID NO: 115 - DNA, 126 bp, HOMO SAPIENS; identical to SEQ ID NO: 27

SEQ ID NO: 116 - DNA, 97 bp, HOMO SAPIENS
gatcacttgagcccagaagttcaaggctgcaatgagccatgattacaccacggcactacaaccttggtggcacagtgagaacctgactcttaaaaar

SEQ ID NO: 117 - DNA, 199 bp, HOMO SAPIENS
gatcagctgaaggttcatgggttttaagtgcttgtggctcactgaagcttaagtgaggayttccttgcaatgagtagaatttcccttctctcccttgtcacaggtttaaaaacytcacagcttgtataatgtaaccatttggggtccgcttttaacttggactagtgtaactccttcatgcaataaactgaaaagagcc

SEQ ID NO: 118 - DNA, 165 bp, HOMO SAPIENS
gaaggaaaatttgtattatttcaattatttttatgtacagaaaactcaacagtgtacatttaacccagtttagtggcaagttctttagcctttgccttttcgagcttggygatacgagccacagacttaggacccaggacattgccaccccagtgacggcggatc

SEQ ID NO: 119 - DNA, 142 bp, HOMO SAPIENS
aattgttgyttctttctagtggtctttgtaagagtgtagaagcattccttctttgataatgttaaatttgtaagtttcaggtgacatgtgaaaccttttttaagatttttctcaaagttttgaaaagctattagccaggatc

SEQ ID NO: 120 - DNA, 111 bp, HOMO SAPIENS
gatcagcttttacagctgcactctattcagctgcagcttcatgctaaaactggtcaaagtgctgactctggtaccattaaggcaaaattggaargagagcttgaggcaaac

SEQ ID NO: 121 - DNA, 103 bp, HOMO SAPIENS
gatcaccgcccagtatgggctagagcaggtcttcatcatgccttgtcttttttaactgagaaaggagattttttgaaaagagtrcaattaaaaggacattgtc

SEQ ID NO: 122 - DNA, 94 bp, HOMO SAPIENS
caatttgagagcaggtactgtttattaaccaaccagcttagaaaaataatcatggymgacaccttagttcattcttctaataagcctgttgatc

SEQ ID NO: 123 - DNA, 94 bp, HOMO SAPIENS
ggttgacaaagagggtatttatttagggtttactgggtacagggagaagggctggatggcttgggatgcagagagagacccttcccctgggatc

SEQ ID NO: 124 - DNA, 86 bp, HOMO SAPIENS
gatcctcgggaagaacaaagctaaagctgccttttgtctgttattttatttttttgaagtttaaataaagtttactaattttgacc

SEQ ID NO: 125 - DNA, 62 bp, HOMO SAPIENS; identical to SEQ ID NO: 62

SEQ ID NO: 126 - DNA, 65 bp, HOMO SAPIENS
gatcataggaatattasgagttacctctctgtggaggtattgacttgtaatctcattttcctttc

SEQ ID NO: 127 - DNA, 58 bp, HOMO SAPIENS; identical to SEQ ID NO: 64

SEQ ID NO: 128 - DNA, 22 bp, HOMO SAPIENS
gatcacagccgaaggagtaaag

SEQ ID NO: 129 - DNA, 219 bp, HOMO SAPIENS
ctttttaacctttatttacaaagaacacaactcctcttgacacaaacacacacacatttcaaggaggagcccattaagaacaacaacagactggcgagaatgacggggccttattgaacactgtcaataaggaacactggtctgtgtaccccagactaggggtcactgcaaacattttctcagatatgacttttcacctccaagacaggaaaagagatc

SEQ ID NO: 130 - DNA, 214 bp, HOMO SAPIENS
agtccatgaggcattttatttgtaaatatatgtattacatccctagaaaaagaatcccaggattttccctcctgtgtgttttcgtcttgcttcttcatggtccatgatgccagctgaggttgtcagtacaatgaaaccaaactggcgggatggaagcagattattckgccatttttccaggtctttgagttgcacgtcaaatctggggctgatc

SEQ ID NO: 131 - DNA, 192 bp, HOMO SAPIENS
gatcaatgccactgacccttctgcccgcacgctgacccactacaagtcagctgtcaaagcttaggtggtctttatgctgtaatgtgaaagcaaatatttcttcgtgttggatggggactgtgggtagataggggaacaatgagagtcaactcaggctaacttgggtgtgaaaaaaataaaagtcctaaatct

SEQ ID NO: 132 - DNA, 190 bp, HOMO SAPIENS
gatcatcaattttgtctcactacctacaaatggaatttcatcttgkttccatgctgagtagtgaaacagtgacaaagctaatcataataacctacatcaaaagagaactaagctaacactgctcactttctttttaacaggcaaaatataaatatatgcactctagaatgcacratggtttagtcactaa

SEQ ID NO: 133 - DNA, 155 bp, HOMO SAPIENS
gatcatagcttgaactgaagccaaggacagagtgctgcccccttcggcatttactgatgtgccctctttaaatcatgatgttatctaacccaaacccagacccaggacctagtcacagctccaacctacacttcctattaatcttaaaacaaagc

SEQ ID NO: 134 - DNA, 149 bp, HOMO SAPIENS; identical to SEQ ID NO: 12

SEQ ID NO: 135 - DNA, 145 bp, HOMO SAPIENS; identical to SEQ ID NO: 15

SEQ ID NO: 136 - DNA, 143 bp, HOMO SAPIENS
gaggaattaacagtctttattgggctcagaccaggagtccgtgggtcttgaggacctctgtgtatttgtcaattttcttctccacgttcttctcggcctgtttccgtagcctcatgagctgtttcttcttccggtagtggatc

SEQ ID NO: 137 - DNA, 138 bp, HOMO SAPIENS
actgaatgacgtttaatgaacaaatgaggaattggttatttatactctgtggacgtttattaaggctctggcaacactgtggagctggcctgygtccccgagaagaagagtcagtcacaggggggtctccatctgatc

SEQ ID NO: 138 - DNA, 137 bp, HOMO SAPIENS; identical to SEQ ID NO: 22

SEQ ID NO: 139 - DNA, 135 bp, HOMO SAPIENS
ggtttatttaaaaatagaaaacagcaacctttttttgttttatagcctacttctcaaaattgtygtgtgattagtgacaacgggggaatctacaatgctcacatcacagtaaactaccactgacttaytaggatc

SEQ ID NO: 140 - DNA, 133 bp, HOMO SAPIENS; identical to SEQ ID NO: 23

SEQ ID NO: 141 - DNA, 133 bp, HOMO SAPIENS
gtgtttactacaaactgtttaattgtttcttatcccaataactttacaaatatagaaccacatgctaktctgggggtgctgtgcagtgagtcactacaaactcgctcaggcacagcttaatgccgctgagatc

SEQ ID NO: 142 - DNA, 121 bp, HOMO SAPIENS
gatccaataacaaatacctatatgctctttacttattttatcagttgataaaattttgtgtcattggtttatctgtttatcaactttttggggggaggaattttaaagtgatgataccaac

SEQ ID NO: 143 - DNA, 120 bp, HOMO SAPIENS
aacagtttctaaaacatttttattgtaaaaagttcaagaagccatttacaagccaaaaagtatcagaattaaataacacataatttttatagacacatttttctgtacaaagggctgatc

SEQ ID NO: 144 - DNA, 117 bp, HOMO SAPIENS; identical to SEQ ID NO: 31

SEQ ID NO: 145 - DNA, 113 bp, HOMO SAPIENS
gctgtcttccggaaccttctcttcctgacattctttagaggcagagtaattccgtggttccagataaactttttctctttgtccttatcctttttcttgcttgctttaggatc

SEQ ID NO: 146 - DNA, 106 bp, HOMO SAPIENS
cacaytacaatagttaattttatttgttcaagagctcagattgcaagcattaaaccaagcataggctttgattctgtgagcccaaattcacatattgaagaagatc

SEQ ID NO: 147 - DNA, 102 bp, HOMO SAPIENS
gcaggatgagacacatttattctacatctggtcacagtaaatctcacccagaccacaggcaggatgccacgtggctacaggcagctggcggaaatggagatc

SEQ ID NO: 148 - DNA, 101 bp, HOMO SAPIENS; identical to SEQ ID NO: 39

SEQ ID NO: 149 - DNA, 101 bp, HOMO SAPIENS
gatcattgttctactttgtatttactactgtgtgaatcagttgattctacttcaggtccttgcttaaattcctgtcactaaatggaaatgtgtttggtttt

SEQ ID NO: 150 - DNA, 97 bp, HOMO SAPIENS
gatcgcaaggctgaggatgccaggagggactatgaaaaagccatgaaagaatatgaagggggccgaggcgagtcttctaagagggacaagtcaaaag

SEQ ID NO: 151 - DNA, 95 bp, HOMO SAPIENS
gatgttttctttttatttttctttggaattgtaaaggagtttactgttttaatacattgttcttctggctgaggctggtcaaagctacactgatc

SEQ ID NO: 152 - DNA, 95 bp, HOMO SAPIENS
aaacctttaatcaattttattcttcatatgtgggtaagggtacagattctagaccatgtctgtgaagtcatggtgatatcactgtgctaaagatc

SEQ ID NO: 153 - DNA, 94 bp, HOMO SAPIENS
gatcatatgctgtttgtagccataaggtaaatcatgtctcttccaatcatgactttggaactccctgaataataaaaatgagagttgagaaaac

SEQ ID NO: 154 - DNA, 92 bp, HOMO SAPIENS; identical to SEQ ID NO: 44

SEQ ID NO: 155 - DNA, 79 bp, HOMO SAPIENS
gctcaggactatttrctttcagagcacaaaacaggttacagacaggtgtgtgccaggagtcgcaagatttggctggatc

SEQ ID NO: 156 - DNA, 77 bp, HOMO SAPIENS
gatcttgtacccgctaaaactgcacagctcgaccttcatggcgacagctccacggaaaracaaaagatggcgaagag

SEQ ID NO: 157 - DNA, 74 bp, HOMO SAPIENS
gatcagagggtagtacagaagccctgactcatgccttgagtacataccatacagcaaataaatgktagcaaaac

SEQ ID NO: 158 - DNA, 72 bp, HOMO SAPIENS
gatcttaagattgaaagtgatgttcaagaaccaactgaaccagaggatgaccttgacattatgcttggcaat

SEQ ID NO: 159 - DNA, 72 bp, HOMO SAPIENS
gatcatccttcctggcaaataaattcccgtttctatccaaaagagcaataaaaagttttcagtgaaatgtgc

SEQ ID NO: 160 - DNA, 71 bp, HOMO SAPIENS
gatcttaagycatacattttaattgtgtagaggttgttcaactgaaggaataaatgtctattaaactaaam

SEQ ID NO: 161 - DNA, 66 bp, HOMO SAPIENS
ctattatttgktcttcctcctcatcaccaaaagtctgttcctgcatggtggtaaaggagagagatc

SEQ ID NO: 162 - DNA, 64 bp, HOMO SAPIENS
atgattcaaggatttattaagtcatacatgcaaaacataatgctaattgcattagcaaaagatc

SEQ ID NO: 163 - DNA, 56 bp, HOMO SAPIENS
gacagtttaaacaaaaattttaatttgtggtttatttcataatcccaaactggatc

SEQ ID NO: 164 - DNA, 36 bp, HOMO SAPIENS
gatcacaagactaaagataattaaaaagaaaaccac

SEQ ID NO: 165 - DNA, 33 bp, HOMO SAPIENS
aatctttcaatcttttatttaaatgccatgatc

SEQ ID NO: 166 - DNA, 33 bp, HOMO SAPIENS
cttttgattaaagaagttcaagtcatctagatc

SEQ ID NO: 167 - DNA, 23 bp, HOMO SAPIENS
gatctggcacaagatagaatggy

SEQ ID NO: 168 - DNA, 20 bp, HOMO SAPIENS
cttctgaatttttttagatc

SEQ ID NO: 169 - DNA, 91 bp, HOMO SAPIENS
atttaaaaaaccttcggtgcaatattaaaaagcaatacagccagctggagcgacaatcaacagaaagaaaagaggggcatatttagagatc

SEQ ID NO: 170 - DNA, 68 bp, HOMO SAPIENS; identical to SEQ ID NO: 57

SEQ ID NO: 171 - DNA, 156 bp, HOMO SAPIENS
gatctcaggcatcggattatttcttctgtaaatatttcagaatgtatctctccaagatgagagctcattaaaagataattacaaagcttatcacatccaaaagaattatcaataattttgaaatattattaaacgtgtaataaatgttcaaagttc

SEQ ID NO: 172 - DNA, 155 bp, HOMO SAPIENS
gatccttcattagaaaagtctatacagtttgcattgaggcagaatttacatgaaataggtgagcggtgtgttgaagaactcaagcatttcattgcagagtatgatacttccactcaagattttggagagcctttttagatttttctgctcaggct

SEQ ID NO: 173 - DNA, 86 bp, HOMO SAPIENS
gatcatctgaagaaaattcatcagttttctgcttaactctatatcttatgtgattctgatattacaataaaattatggtaaacttt

SEQ ID NO: 174 - DNA, 36 bp, HOMO SAPIENS
gatcaacaaaattgatagaccgctagcaagactaat

SEQ ID NO: 175 - DNA, 55 bp, HOMO SAPIENS
cacgactccagttactatgatgacaggtcagaccatgagcgagagaggcacaaag

SEQ ID NO: 176 - DNA, 81 bp, HOMO SAPIENS
gcattcaagacaccagcatctgttttatttttgacagcagcgtctggtccttatatattcatctaacactttcagtagatc

SEQ ID NO: 177 - DNA, 232 bp, HOMO SAPIENS
gatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtac

SEQ ID NO: 178 - DNA, 145 bp, HOMO SAPIENS
actgtgaaaatctttcttttattactgtctcatgtggcaaaataacattttatacaaataagtcacctagtttactttggacacaaagacaataaaaactggaatgtgattttggggaagcgggaaggaggaggaaggacagatc

SEQ ID NO: 179 - DNA, 82 bp, HOMO SAPIENS
gatcatcatgtggggattaccattgttcctggagtactcctacccttagttgaatttccttattaaagttatatttttctat

SEQ ID NO: 180 - DNA, 57 bp, HOMO SAPIENS
gtatgaataaatgctttcagttcacttcagtatacacctagaaggaacttcaggatc

SEQ ID NO: 181 - DNA, 53 bp, HOMO SAPIENS; identical to SEQ ID NO: 67

SEQ ID NO: 182 - DNA, 180 bp, HOMO SAPIENS
ttttattttttttgtgcgtgtctaataaccaggaaaaaaataaagcttaggttttaaaaagatttaaaaataatctgtttcagaaactgtcaaatgtaccatatttgtattaagagttgttgggaatttttgtacaatgaatttacatttatttatggtgacatatttacgcttgtgatc

SEQ ID NO: 183 - DNA, 49 bp, HOMO SAPIENS; identical to SEQ ID NO: 70

SEQ ID NO: 184 - DNA, 52 bp, HOMO SAPIENS
gatcaggagttcaagaccagcctggccaacatggtgaaaccctgtctctatt

SEQ ID NO: 185 - DNA, 204 bp, HOMO SAPIENS
gatcagcagccctctggaagtgagggagaggatgatgatgcggaggctgccttgaagaaagaagttggtgacattaaggcatctacagagatgaggttaagaagattccagtcagtggaaagtggagcaaataacgttgtcttcatcaggacacttgggatagagcctgagaaattggtgcatcatattctccaggatatgtac

SEQ ID NO: 186 - DNA, 117 bp, HOMO SAPIENS
gatctggacagcagaatgttataacgcaagttcatgtgttgctcccaactccattctcttttctctcgtgcaaccagtttgcccattctcttcctattacttgctccagggataggt

SEQ ID NO: 187 - DNA, 98 bp, HOMO SAPIENS
gatctaagagttacctggctacagaaagaagatgccagatgacacttaacacctacttgccacattcaaatgatgcaataaaagacctattgatttgg

SEQ ID NO: 188 - DNA, 71 bp, HOMO SAPIENS
gatctgggattttgtttttattttgaaatgggagcttttttgtttacaagttcattaaaaactaaaaactg

SEQ ID NO: 189 - DNA, 91 bp, HOMO SAPIENS
gtttattgggtcagtttatttttctcctttacagatattgaaaactcttgggctgaactgtggaatagtaatttcgaattaaatccagatc

SEQ ID NO: 190 - DNA, 307 bp, HOMO SAPIENS
gatctctggcagtggaggaagtctctttaagaaaatagtttaagcaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgccgtttagtttttaaagatggaactccaccctttgcttggttttaagtatgtatggaatgttatgataggacatagtagtagcggtggtcagacatggaaatggtggggagacaa

SEQ ID NO: 191 - DNA, 134 bp, HOMO SAPIENS
aacacttctactaacagctggcttctacaaagacttaaaatattaggatatttgactcttctgaggtgtgttcacatgctactggtactcagagattgctttgtcaccttggggttattttcaagcctacgatc

SEQ ID NO: 192 - DNA, 124 bp, HOMO SAPIENS
agattttcttcctcctcaaaacttccatttctattgtaatattcttaatgacaatctttttttttttttagcagtgtatgtttgaaacagccaaagatggcgatgaaccaagtgtaaattgatc

SEQ ID NO: 193 - DNA, 56 bp, HOMO SAPIENS
aagaggctcccattatgttgcccaggctggatttgaactcctgggctcaagcgatc

SEQ ID NO: 194 - DNA, 234 bp, HOMO SAPIENS
gatcaccgtgacatccgaggtgcctttctccaaaaggtatttgaaatatctcaccaaaaaatatttgaagaagaataatctacgtgactggttgcgcgtagttgctaacagcaaagagagttacgaattacgttacttccagattaaccaggacgaagaagaggaggaagacgaggattaaatttcatttatctggaaaattttgtatgagttcttgaataaaacttgggaacc

SEQ ID NO: 195 - DNA, 68 bp, HOMO SAPIENS
agtacagccaaagttttaaatacagaaagcacaagaataaaccaatggtaacatgtagaatctagatc

SEQ ID NO: 196 - DNA, 74 bp, HOMO SAPIENS
gatccaccacccagcttcacccctcacccccagcggctcaccatggggatggcagcaataaaatattttcccac

SEQ ID NO: 197 - DNA, 46 bp, HOMO SAPIENS
gatcctgatttaagccatggattaaaattataatccattaagagac

SEQ ID NO: 198 - DNA, 141 bp, HOMO SAPIENS
ccaaatgtgatgggtttattttattttactatgatgcaaaaccaaagattccactatagcacagagaccagtatactacaaggtcatgaaaaaatggtgtgggacgtgcaggacgtgatgtacaaactggtgggtcagatc

SEQ ID NO: 199 - DNA, 148 bp, HOMO SAPIENS
ttaatttatgaaggcatcatgtcactgcaaagccgttgcagataaactttgtattttaatttgctttggtgatgattactgtattgacatcatcatgccttccaaattgtgggtggctctgtgggcgcatcaataaaagccgtccttg

SEQ ID NO: 200 - DNA, 226 bp, HOMO SAPIENS
gatccatcatccgcaatgtaaaaggccccgtgcgcgagggcgacgtgctcacccttttggagtcagagcgagaagcccggaggttgcgctgagcttggctgctcgctgggtcttggatgtcgggttcgaccacttggccgatgggaatggtctgtcacagtctgctccttttttttgtccgccacacgtaactgagatgctcctttaaataaagcgtttgtgtttc

SEQ ID NO: 201 - DNA, 35 bp, HOMO SAPIENS; identical to SEQ ID NO: 78

SEQ ID NO: 202 - DNA, 47 bp, HOMO SAPIENS
gatctcaccactgcactccagcttgggggacagggcaagtctgtctc

SEQ ID NO: 203 - DNA, 103 bp, HOMO SAPIENS
gatcgcgaataaaaatcaacaaatgtgaaagcccagaaaaatatattcgtatttctggttttgctggatttttacatttttatataataaaaatgttattttg

SEQ ID NO: 204 - DNA, 93 bp, HOMO SAPIENS
gagacagggtctcacttgctctgtcacccaggctggagtgcagcggtgcaatctcggctcactgcagccttgacctcccaggctcaagagatc

SEQ ID NO: 205 - DNA, 41 bp, HOMO SAPIENS
aagctgtaattttcacaatattttaattctgttctgagatc

SEQ ID NO: 206 - DNA, 162 bp, HOMO SAPIENS
cgggttttttgctttatttattcagagagtgaaactgtgttacagtgagctaaatttttagctgtacttagaatgtgctccctcctacatttcactctggaaaagtgaagtatcttatcatttcttagaaacctggatgtgaaactagtcaatattaagatc

SEQ ID NO: 207 - DNA, 162 bp, HOMO SAPIENS
gatcacgttaggtcagacgtttgagaccagcctgacccacatggagaaatcctgtctctactcaaaatacaaaattagccgagcatggtggcacatgcctataatcccagctacatcgccccattgcactccagcctggaaaacaagagcaaaactctgtct

SEQ ID NO: 208 - DNA, 46 bp, HOMO SAPIENS
gcaaaaaaatcaagtttttatttcaaatatttgaatctaatagatc

SEQ ID NO: 209 - DNA, 81 bp, HOMO SAPIENS
ccgagtcaatattttattaaaaaacacacaaacacatgcacactggtaacaaaggcagagatggagaggagacacaagatc

SEQ ID NO: 210 - DNA, 50 bp, HOMO SAPIENS
gagacagggtcttgttctatcaccccaggctggagtgcagtggtgtgatc

SEQ ID NO: 211 - DNA, 154 bp, HOMO SAPIENS
gatcctgctgctgtaatgggaacccctcccccatttacttctccacctcccgtcctccccatcattggtttttttttgtgtgtcaactgtgccgtttttattttattccttttattttcccccttttcacagagaaataaaggtctagaagtag

SEQ ID NO: 212 - DNA, 88 bp, HOMO SAPIENS
accaatgaggcaatttattaacccagcatggtttgttctaatgcttcttgttggcagctgccacctgtccggcgattctgtccagatc

SEQ ID NO: 213 - DNA, 151 bp, HOMO SAPIENS
ttttttgtgagtgctaaattggcatcatatgtcaagttttatcagaataataaagacttcattgattcacttggcctgttacatgaacagaatatggcaaaaatgagactacttacttttatgggaatggcagtagaaaataaggaaattt

SEQ ID NO: 214 - DNA, 120 bp, HOMO SAPIENS
ggttttaaacctttaatgagaaaaaaatatataataccgagctcaaaaacactgtgtttggtgtcattgggtcaagggttggggatacacacggcagaattcggaaacagggtatagatc

SEQ ID NO: 215 - DNA, 51 bp, HOMO SAPIENS
gggatacacattcgcttttaataaaaaagaaatttcctcctacaattgatc

SEQ ID NO: 216 - DNA, 203 bp, HOMO SAPIENS
gatcttctccctagaaaaaaatggacatatgtacatgcacacaaacccacaatttgtgtgtactctcaaaaatctgggctccaggttaaaaattcctatgttaagggaaaagctagtcactttataaaggttaagggtgtaaaagtatattaagtttcccttgcacatgatgcatcatgcaggaatttaacaacaacaacaaa

SEQ ID NO: 217 - DNA, 75 bp, HOMO SAPIENS
attttatttatttatttgtttattttgagacagagtcttgctctgttgcccaggatggaatgcagtggtgtgatc

SEQ ID NO: 218 - DNA, 64 bp, HOMO SAPIENS
gatcgtaatgtttatcatgttacttccccacccctacattttttgaaataaaataaggaatttt

SEQ ID NO: 219 - DNA, 45 bp, HOMO SAPIENS
gatctgcatgtttgcgtcagctcggccgcctcgctgaagaaaaag

SEQ ID NO: 220 - DNA, 102 bp, HOMO SAPIENS
gatcaggagcgtctgggcaagcagctcaagttacgtgcagaaagggaagaaaaggagaagctgaaagaggaggccaagcgggccaaggaggaggccaagaag

SEQ ID NO: 221 - DNA, 18 bp, HOMO SAPIENS
gatcaaagcatcaagatt

SEQ ID NO: 222 - DNA, 45 bp, HOMO SAPIENS
gatcttggaactcccaactttaatttggtgtaataaaaatgatgc

SEQ ID NO: 223 - DNA, 206 bp, HOMO SAPIENS
gagacagagtctcactctgttgtccagcctggagggcactggcaccatctcagcttccaggttcaagcaattctcctgcctcagcctccatagtggctggggttacaggtcgcgccactgtgcctagccaatttttgtgtttttagtagagatggggttttaccatgttggccaggctggtcttgaactcctgtcctcaagtgatc

SEQ ID NO: 224 - DNA, 53 bp, HOMO SAPIENS
acagttattttgcttttaataaagtaaatgtaatgacaggagtagggaggtga

SEQ ID NO: 225 - DNA, 112 bp, HOMO SAPIENS
gatcttgatggtgtttctttccccaaaaattgacttagatattaaaatttggtgcttataagagagagttaaaaaaaaataggattgcttcaattaaaattacaaaagagac

SEQ ID NO: 226 - DNA, 128 bp, HOMO SAPIENS
gatcaagcgtgctttccttatcgaggagcagaaaatcgttgtgaaagtgttgaaggcacaagcacagagtcagaaagctaaataaaaaaatgaaacttttttgagtaataaaaatgaaaagacgctgt

SEQ ID NO: 227 - DNA, 38 bp, HOMO SAPIENS
agcttttgcttattttattcaatatatttgaaatgatc

SEQ ID NO: 228 - DNA, 54 bp, HOMO SAPIENS
ttttttgttggcgggggagatggtactataacttgttatttatcagggcagatc

SEQ ID NO: 229 - DNA, 72 bp, HOMO SAPIENS
ggagaccatactgacttgattattcttagaaatgcacttgacctgcttttgccaaagcttgccagagtgatc

SEQ ID NO: 230 - DNA, 67 bp, HOMO SAPIENS
gatcaatgaaccatctcttctgggcagttttgttgaaaataaaggtttttctttgatttcaagaatg

SEQ ID NO: 231 - DNA, 48 bp, HOMO SAPIENS
gatcacgtcactgcaccccagcccaggcaacagtgtgagaccctgtct

SEQ ID NO: 232 - DNA, 223 bp, HOMO SAPIENS
gcttttaaaatatttattgagcagagaaaaaggaagggagctaaaaagatgaaatagatgttgagttaacagctttcttccaagttctggtggacttcagactttaaatgtgtggctcaaacttcaattctccagctggccctgatggaggaattccttgttgtaattttttttgttccatctgctgtatgacctgatgatattccagcttctgagcctccag

SEQ ID NO: 233 - DNA, 49 bp, HOMO SAPIENS
gatcacaccactgcaccccagcctaggtggcagagggagaccttgtctc

SEQ ID NO: 234 - DNA, 154 bp, HOMO SAPIENS
agtaagatactgagataaaaataatttaaaaaaatccaggatgaacaagtttcaaaatgcatagtgttctgtgcatgagtccattttctttaaactctgaaagaataaagtggtaagaaaacaagaaaaaacactgctgctgcattctctgatc

SEQ ID NO: 235 - DNA, 225 bp, HOMO SAPIENS
gatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaag

SEQ ID NO: 236 - DNA, 36 bp, HOMO SAPIENS
tttgccaaataatatttctccttttatagatggatc

SEQ ID NO: 237 - DNA, 246 bp, HOMO SAPIENS
gatagtaaaataattttatttagtttcttattttaaaaaaggtagcatttcaacatataaatttagactcaagattacagtgtatatttgccaaaaggaacaccccgaaagacggccagcctcagagctgagagggcacagggaggcacactcctcacacatgggagtttaagcagaagtgctttaagacttcacagcggcatttccctgttcctcagccccgcctccagggccatcactttgggg

SEQ ID NO: 238 - DNA, 73 bp, HOMO SAPIENS
gatcccaggaattctaatgtgagagactcctggccaaccccaggcattctaataaagagacttctgtctgacc

SEQ ID NO: 239 - DNA, 256 bp, HOMO SAPIENS
ttatactgattttgatatcatgttcttttcatagtcgtattttgccaccgtaaacataaaaaaaaaaaaaaagatttccaaaatgccgttttcagaacctgggttttaatagcagtattgaatttgtaagcttagtagttgcagaaattgaacactaggtggcactcagttatcttaacaggggaagtactgatacaattgttgacttttcttttactatgtgtaagaaataccccaaacatgaaaagattgtttt

SEQ ID NO: 240 - DNA, 176 bp, HOMO SAPIENS
gatcgggctactacaaagttctgggaaagggaaagctcccaaagcagcctgtcatcgtgaaggccaaattcttcagcagaagagctgaggagaagattaagagtgttgggggggcctgtgtcctggtggcttgaagccacatggagggagtttcattaaatgctaactactttttc

SEQ ID NO: 241 - DNA, 166 bp, HOMO SAPIENS
  misc_feature (116)..(116): n = a, g, c or t
cgatcatatgggatgtctgaagaaatatgaaactgtgcaagacattctgaaagaattctttgatttacgattaagttattacggtttacgtaaggagtggcttgtgggaatgttgngagcagaatctacaaagcttaacaatcaagcccgtttcattttagagaag

SEQ ID NO: 242 - DNA, 67 bp, HOMO SAPIENS
ggttttgtagagatggtatgtcactattgtgaccaggctggtctcaaactcctgacatcaagtgatc

SEQ ID NO: 243 - DNA, 231 bp, HOMO SAPIENS
gatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttc

SEQ ID NO: 244 - DNA, 169 bp, HOMO SAPIENS
gttctggtgttaaagaacccagtataaggcaagaagaaaaatcagctaaggagaacttctgcttcctgtcaagatggagaaacaggaacagtttactcttccatccttttcattttcagagtgtttcttggtttccaaagagcttccacatctaagctcccatttgatc

SEQ ID NO: 245 - DNA, 62 bp, HOMO SAPIENS
ggtgaaaagatatatatatatatatattcagaattaggcagctggactcaatttagatgatc

SEQ ID NO: 246 - DNA, 155 bp, HOMO SAPIENS
tcttttttccggcgttcaagatgtcgaagcgaggacgtggtgggtcctctggtgcgaaattccggatttccttgggtcttccggtaggagctgtaatcaattgtgctgacaacacaggagccaaaaacctgtatatcatctccgtgaaggggatc

SEQ ID NO: 247 - DNA, 53 bp, HOMO SAPIENS
gatcaggatgccactgcactccaacttgggcgacatagtgagactacgtctcc

SEQ ID NO: 248 - DNA, 231 bp, HOMO SAPIENS
ggagaaacgttgtgtttaatggtaaagcttaacacaccccagcaccaagaatggtgtggagttgcagtagcaacaggccggtgaccccccatggagcctcacatggcgaagaggatgaggaaggcgaccatcaaacagaaaagccccatggcctcagacagggcaaagccaagaatggcataggagaagagctgctgcttgagagacgggttcctggcatagccaatgatc

SEQ ID NO: 249 - DNA, 148 bp, HOMO SAPIENS
gagtttttacgggtgttcatttattgactgctgggaatacagcctattgagaatacatggcatgcatttggtggagaaatcaactaaatctgaaatgaagccattcagtaagcctgctgtgagttaataaggtagcacagattagatc

SEQ ID NO: 250 - DNA, 53 bp, HOMO SAPIENS
attttacagcacaagctttatgaggaataggagaacacatttttttcacatta

SEQ ID NO: 251 - DNA, 188 bp, HOMO SAPIENS
gatctagggcaggctgtcttccagtccatgtgttctcggtcgccgtagacagcgctctggctaccaccgtgaggctacttgaactgtcaggggcatctgcctaaaccagaatcttttgtcagaaaccttaacccaacaaaacaaatcttgagtagctcatgcccggctcttaagaattttgtctgttt

SEQ ID NO: 252 - DNA, 65 bp, HOMO SAPIENS
gatcttagttgcttatttgttttgactcttgtgtgctgtgggcactggagtagagatttctggag

SEQ ID NO: 253 - DNA, 36 bp, HOMO SAPIENS
gtattggttttatttaaattttacagaaacctgatc

SEQ ID NO: 254 - DNA, 168 bp, HOMO SAPIENS
ctagtaattcttatggtattgctgggtctctcaggaatatgtatcatttgattttgagcatgtggggttaaggtattagattactaccacaaaccgtagacccctgcatggcaccacatttattttcaggagtagatgttacatggcaggtatcaaaatgtgatgatc

SEQ ID NO: 255 - DNA, 106 bp, HOMO SAPIENS
gctttgaaaggaaagtttattatttcccaatgttctttacattttcatttggaaatatcattcctgacagaaatagatacattataccttcgaaagcagaaagatc

SEQ ID NO: 256 - DNA, 144 bp, HOMO SAPIENS
gatcattaaatgaaagatttttgaaacagatgtttatgtaaaatgtagcaatattttatgcttatggtccagtatttcattcagtaactatcttgggggtcctttttacttgaacctctagtaattcttgagctgtctcttggg

SEQ ID NO: 257 - DNA, 62 bp, HOMO SAPIENS
gctagttcagtgaaacagagggaatgaagaagaaactaagcaatctgtaactggttgtgatc

SEQ ID NO: 258 - DNA, 76 bp, HOMO SAPIENS
gatctaaggacttcgatggttccagccttcaaagaaaaaataatttaataaaaatttagttagaaaaaataacccc

SEQ ID NO: 259 - DNA, 94 bp, HOMO SAPIENS; identical to SEQ ID NO: 43

SEQ ID NO: 260 - DNA, 72 bp, HOMO SAPIENS; identical to SEQ ID NO: 54

SEQ ID NO: 261 - DNA, 162 bp, HOMO SAPIENS
gatcgagtcaagatgagttagtggagctgggcttggccagggagtctggggacaaggaagcagattttcctgattctggctctagcttccctgccaagattttggtttttatttttttatttgaactttagccgtgtaataaactcaccagtggcaaaccag

SEQ ID NO: 262 - DNA, 66 bp, HOMO SAPIENS
gatctctttgtagccatcctgttaaatttgtaaacaatctaattaaatggcatcagcactttaacc

SEQ ID NO: 263 - DNA, 81 bp, HOMO SAPIENS
gtgtttataacaggttttactttttttcttaaaatagagatttcttactaaataccattttatttcatttcttcacagatc

SEQ ID NO: 264 - DNA, 90 bp, HOMO SAPIENS
cagtttattgtatacacacacatttcactggtggttgttttttttaaaaagtctgtacaattgacaagacaatacaaactagctacgatc

SEQ ID NO: 265 - DNA, 39 bp, HOMO SAPIENS
gatctgaaagaggtccagaataaaatatattttgatatt

SEQ ID NO: 266 - DNA, 124 bp, HOMO SAPIENS
gaagaggcagaccagctttatctttgaacccaatcactgggtgcccaactataactctgctatcttcgaaatgatgagatggggcacactcccctaacagtatttagctagaccaacaaggatc

SEQ ID NO: 267 - DNA, 23 bp, HOMO SAPIENS
gcaatgtttagaacattttatta

SEQ ID NO: 268 - DNA, 28 bp, HOMO SAPIENS
ctccaaggagtagctttattcagggatc

SEQ ID NO: 269 - DNA, 134 bp, HOMO SAPIENS
gatcaaggtagtcttagttttaaaaagaatacacatggaaataaggagaggataaaattaattgccgagttccacttgaatgaccattcttttattttatgaaaatacagttaagtacattatattctttcttt

SEQ ID NO: 270 - DNA, 200 bp, HOMO SAPIENS
gaccaaaggtatattaatatattgattctttgaaaatgagaattgagacatgggctaaaattaaatacatcaacaacaaaaatcttgtaaggagaggttaaaagcagttatcttcaaaaagtaggtctgagtttatagagtggaggtaacttatttgttatgagctctttaggataaggtatatttgtttcattttgatc

SEQ ID NO: 271 - DNA, 157 bp, HOMO SAPIENS
gatcgctccctctgtaaggaacaaaacgttctcagctgatgtcacggtaggtttagattctgtagagtgttccccaacccgcaccgttctgtacctctcacaccactgcttgcccgggcagtagtggctgaccaataaagacctttacctgttttgt

SEQ ID NO: 272 - DNA, 45 bp, HOMO SAPIENS
gagacagagtctctgttgcctaggctggagtgcagtggtgtgatc

SEQ ID NO: 273 - DNA, 54 bp, HOMO SAPIENS
gatcggacaaagggtttagtaagcaactcaaaataaagtcatttgaattgcctc

SEQ ID NO: 274 - DNA, 125 bp, HOMO SAPIENS
gcaggttagcattttattagataaaaacaggaaggagggtgcctacccacccctgcctgctactggctctgaagggcactcctcaacttccccaagaaagaggacgcgtctctgacactgtgatc

SEQ ID NO: 275 - DNA, 48 bp, HOMO SAPIENS
gatcgcgccactgcactccagcccggtgacaaagcgagactctgtctc

SEQ ID NO: 276 - DNA, 43 bp, HOMO SAPIENS
gatccagctcagaacgattggaaataaatttgaaatgtaaccg

SEQ ID NO: 277 - DNA, 71 bp, HOMO SAPIENS
gagacagggtcttgctctgttgcccaggctggagtacagtggtgtaatcatagctcactgccgcctagatc

SEQ ID NO: 278 - DNA, 254 bp, HOMO SAPIENS
gatccctttatgaaccttgtgatagatgaatgtgtggagatggcgactagtggacaacagaacaatattggaatggtggtaatacgaggaaatagtatcatcatgttagaagccttggaacgagtataaataatggctgttcagcagagaaacccatgtcctctctccatagggcctgttttactatgatgtaaaaattaggtcatgtacattttcatattagactttttgttaaataaacttttgtaatagtc

SEQ ID NO: 279 - DNA, 51 bp, HOMO SAPIENS
gatcacaacactgcactccagcctgggcgacaagcaagactccgtctcaag

SEQ ID NO: 280 - DNA, 17 bp, HOMO SAPIENS
agatttgcattccgatc

SEQ ID NO: 281 - DNA, 51 bp, HOMO SAPIENS
gatcataaattaatcaaaatgcactttttaataaaaactgatacttaaaat

SEQ ID NO: 282 - DNA, 148 bp, HOMO SAPIENS
ggtttaaatactgcactctattttttaaggactctgtgcatggtatttccggtggcagatgtgtaagattttttatgtaaatcgtgttgttttagcagaggactatcagacctgacagcagctggacatcgaggtcctcactggggtg

SEQ ID NO: 283 - DNA, 89 bp, HOMO SAPIENS
gatcacaccgcagacatttagatttttatacccaaggcactttaaaaaaatgttttataaatagagaataaattgaattcttgttccat

SEQ ID NO: 284 - DNA, 268 bp, HOMO SAPIENS
ccaagccaaactgtatccagctttattaaagatactttccataaacaatcatggtatttcaggcaggacatgggcagacaatcgttaacagtatacaacaactttcaaactcccttcttcaatgggctaccaaaaatcagaaagccactataaaacccaatgaagtcttcatctgatgctctgaacagggaaagtttaaagtgagggttgacatttcacatttagcatgttgtttaacaacttttcacaagccgaccctgactttcag

SEQ ID NO: 285 - DNA, 231 bp, HOMO SAPIENS
gtgaatgtaaaacatttaatttaaaaatgttgatactacaatatataaaatagctattataaatgcacatagtgtattctatagctgccaggtttacttttttttttttttaaaggaaactgtaagttacactgtggttaagacttgcatcttcacccttgaaaaagcccacattctatcacagtgatgtatggtcagacttaacagccccaattgttaaacacttggatc

SEQ ID NO: 286 - DNA, 178 bp, HOMO SAPIENS
gctctggaagctttttattgttgctccaagtttaagaattttcactgatggcagaaaaccaacacttttagacaatctcccgcatagccctgcccagcttctgaatcttggtcttgacagacatgtcaacatatttctcgccaatgcgcacaatcattccacccaagattgacggatc

SEQ ID NO: 287 - DNA, 124 bp, HOMO SAPIENS
gatctaaagtgaccttgatggacagtggaagaaattacaacatggaattcctcgaataacaatttattgactttaaataattttgtctaatgctacatatacacaattaaaaaacctttacact

SEQ ID NO: 288 - DNA, 205 bp, HOMO SAPIENS
gatccacttctgtgattaagtaaatggatgtctcgtgatgcgtctacagttatttattgttacatccttttccaggcactgtagatgctataataaaaatagctgtttggtaaccatagtttcacttgttcaaagctgtgtaatcgtgggggggtactatctcaactgcttttgtattcattgtattaaaagaatctgtttaaac

SEQ ID NO: 289 - DNA, 133 bp, HOMO SAPIENS
gatctggtctagttaacctagaagtatttttgtctcttagaaatacttgtgatttttataatacaaaagggtcttgactctaaatgcagttttaagaattgtttttgaatttaaataaagttacttgaatttc

SEQ ID NO: 290 - DNA, 58 bp, HOMO SAPIENS
gatcgttctcaggccctccccccggagtacttcagaatgcaataaatcaaaataatgg

SEQ ID NO: 291 - DNA, 134 bp, HOMO SAPIENS
gatctcaggggaagggaggggttactgctgtatcagccacaacctttgcaatccatagattctccatcaggtataaccaagtgttttaaaatcagtccaagaagtgaggtattttgtaccttattttcatcatt

SEQ ID NO: 292 - DNA, 75 bp, HOMO SAPIENS; identical to SEQ ID NO: 50

SEQ ID NO: 293 - DNA, 94 bp, HOMO SAPIENS
gatcctagcacatacttgtgtgtatatacagacatgttacagtgccaagtgtacttcctactgtggtgtgatttaaaaaattgaaataccctat

SEQ ID NO: 294 - DNA, 154 bp, HOMO SAPIENS
cacgactaagagccttctgagcccagcgacttctgaagggccccttgcaaagcaatagggcttctgcctaagcctctccctccagccaataggcagctttcttaactatcctaacaagccttggaccaaatggaaataaagctttttgatgcag

SEQ ID NO: 295 - DNA, 49 bp, HOMO SAPIENS
gatcatgccactgcactccagcctaggcaacagaatgagactccatctc

SEQ ID NO: 296 - DNA, 64 bp, HOMO SAPIENS
tttttggttaaacaatcattttattgcttgagtacacagacaaatttatgcgaccagcgagatc

SEQ ID NO: 297 - DNA, 174 bp, HOMO SAPIENS
tttgggttttttcttaaacctcccttaaagattctttgatgctttgctctatcactgtagacctggtctttttccccccagctttttcttttttacattctgggttgctattttcagattaataatttgatgaccccatcacagtaccaaaataccccccaaaatgaagttcaa

SEQ ID NO: 298 - DNA, 127 bp, HOMO SAPIENS
atgtctagcttctgagtctagatgaaagacagtatgtttcagagaacattggatatcagtttttcccacagcagggactgtgagagacaaccagcagcatcctctttgtaatcacagggcagggatc

SEQ ID NO: 299 - DNA, 16 bp, HOMO SAPIENS
ggcagagatttagatc

SEQ ID NO: 300 - DNA, 151 bp, HOMO SAPIENS
gatcaaattgtgcagtactttgtgcattctggattttaaaagttttttattatgcattatatcaaatctaccactgtatgagtggaaattaagactttatgtagtttttatatgttgtaatatttcttcaaataaatctctcctataaacc

SEQ ID NO: 301 - DNA, 131 bp, HOMO SAPIENS
aaatacaggcttttttttaatctgtaattgatttacatttgattttaagaagacactgtttacatcaggtcaccacaaagcagatttacatacaaccttcaattgcggaaaaacaaagtttctttttgatc

SEQ ID NO: 302 - DNA, 89 bp, HOMO SAPIENS
gatcatctaattctctgtggaatgaatacacacatatatattacaagggataatttagaccccatacaagtttataaagagtcattgtc

SEQ ID NO: 303 - DNA, 160 bp, HOMO SAPIENS; identical to SEQ ID NO: 8

SEQ ID NO: 304 - DNA, 59 bp, HOMO SAPIENS
agagacagagtctccctatgttgcctaggcttgtctcaaactcctggactcaagtgatc

SEQ ID NO: 305 - DNA, 65 bp, HOMO SAPIENS
ggttttacatttaagcttttaatccatcttaaattggtttttgtatgtggtgtaaggtagagatc

SEQ ID NO: 306 - DNA, 59 bp, HOMO SAPIENS
gatccagctgtgcttaagagccagtaatgtcttaataaacatgtggcagcttttgtttg

SEQ ID NO: 307 - DNA, 49 bp, HOMO SAPIENS
gatctcaccactgcactccagcctgggcaacagagtgagactcagtctc

SEQ ID NO: 308 - DNA, 70 bp, HOMO SAPIENS
gctttaaactggggctttatgtggccaggtacaagcccaggcagctctgtgactttcccgcgccgggatc

SEQ ID NO: 309 - DNA, 151 bp, HOMO SAPIENS
gttttaactttttattaaaatgcttaggatacagattgactttctttcgtaaatgactgttttacttttcctgaagtaggacatatatgcactctgataaaacagaatgagaagtcataattcatgggaattcctatacaaggtgctgatc

SEQ ID NO: 310 - DNA, 139 bp, HOMO SAPIENS
tttttgctgtaggcccgggtggttgctgccgaaatgggcaagttcatgaaacctgggaaggtggtgcttgtcctggctggacgctactccggacgcaaagctgtcatcgtgaagaacattgatgatggcacctcagatc

SEQ ID NO: 311 - DNA, 23 bp, HOMO SAPIENS
tcctcttgttgctcccacagatc

SEQ ID NO: 312 - DNA, 94 bp, HOMO SAPIENS
gatgcacccaccttggccttccacaatgctgggattaccacgcccagcctgaaattttaatatacatactatttttatactgatgataacagac

SEQ ID NO: 313 - DNA, 35 bp, HOMO SAPIENS
gctttgaaatttagaaacaaattttatttaagatc

SEQ ID NO: 314 - DNA, 69 bp, HOMO SAPIENS; identical to SEQ ID NO: 56

SEQ ID NO: 315 - DNA, 22 bp, HOMO SAPIENS
gatcagtatttcaaccagatgg

SEQ ID NO: 316 - DNA, 23 bp, HOMO SAPIENS
gatcgttttaaagttatgaaggc

SEQ ID NO: 317 - DNA, 164 bp, HOMO SAPIENS
aggtgtcagtacttttaatgttgtgtacatcaaattacagtacaaagatgactataaacaagatgcagccctcggtttccatgaacagcacactgttacagtaaaccaagtttatattccaccatcaagtgtggctctcccatgacttcgctttgtgatggatc

SEQ ID NO: 318 - DNA, 110 bp, HOMO SAPIENS
gtttgaagttaactcattttatttctaggatttggatttcaacattttaatttctttggaatataagtcactttttgcaagctaaaaaatagaatcaaactaaggtgatc

SEQ ID NO: 319 - DNA, 100 bp, HOMO SAPIENS
ggttattttagagtttgcagtatacatttatagttaatctaagatgactttcaaacaatagtacttgctttattgtgatatttgccttcttgtggtgatc

SEQ ID NO: 320 - DNA, 54 bp, HOMO SAPIENS
gatcacttgagaccaggaattcaagaccagcctgggtacaagtgagaacctgtc

SEQ ID NO: 321 - DNA, 219 bp, HOMO SAPIENS
ccccgaagtcaagtcttaaagcatgttggatttcagaaacatgagtttattcatatcttctggactgagcagccgcctccgtttgattagaagcgcttgttcacacatatttacacacccgcttctggcgcccacggccggaaccgccaggagccagaaggcgagcttggcgagttttgtgtgcttttgggtaacgcacgaccagtactggaagagatc

SEQ ID NO: 322 - DNA, 93 bp, HOMO SAPIENS
cagctcacaagacagttttattgaattagttgcatgcaggagcaattctgttcttcccatgagcagcagagtcgagtgttagagtgcaggatc

SEQ ID NO: 323 - DNA, 267 bp, HOMO SAPIENS
gatcatgttctgtacatgactacaaatagtccgaacggtagccagttcctttctgttaccccaccatttgtcaacccggagcctcttttttttctttccaagaaggctgagttctacattgatgtgattgaagtccctccgcagggttcctctggggcccttcacgataactgtgcgtcccttcagagtaatgtcgacattttctggaatgtcgacagtctgattgctgagaatagtcttcattctcgcagtagacgcagcaaaaaa

SEQ ID NO: 324 - DNA, 91 bp, HOMO SAPIENS
gatctttaagtatacctcttccccagtttggggaggaaaggactcagtttgcaccctttttgtatgtaaaataaaatgtcttacctttctt

SEQ ID NO: 325 - DNA, 170 bp, HOMO SAPIENS
aacgaagaagtgagtattttattattttgctgtacagctgttgcttcactatataaaaacagcaccagcaaatgcagtgtattgcaaaattaagatagtgttgttcttcatctgacactgtacaagcaacaaaaacttcttcactcccagttatttccaatggaaagatc

SEQ ID NO: 326 - DNA, 215 bp, HOMO SAPIENS
accaatagtgccaaaacatttactgaaagttttcaaatcttgcaaaaatgttacaaaaagtgagcaaatgtggtgaaactaatgacaaattagacattgaaaaccaacatctggttaagtgtaggcaggcatgtaagggaaagacaacgtccataggcagttactatggtaaaggactgcatttgactcttttacacatattatttcatttgatc

SEQ ID NO: 327 - DNA, 94 bp, HOMO SAPIENS
aagtagctaagaacaattgctctgaagacgcaaagctaaactgctaacatccaaacacggcctgacagtgtaactgcaaactctgctcctgatc

SEQ ID NO: 328 - DNA, 131 bp, HOMO SAPIENS
ggtaattaagaatttattagacttgaaatagtcaaaagatgagagataatttgtttcatttctattcccttttctgtaaccaagatgaaggccaaagttctcgtttgaatatttgctactaccaccaagat

SEQ ID NO: 329 - DNA, 171 bp, HOMO SAPIENS
gatccgcctggcagccattgcagagtcaggggtagagcggcaagtacttttgggagaccagatacccaaattcgccgttgccactttaccacccgcctgaatcctgggattctagtatgcaataagagatgccctgtactgatgcaaaatttaataaagtttgtcacagag

SEQ ID NO: 330 - DNA, 199 bp, HOMO SAPIENS
cttttattagtttttattagtttttcaaaagtatcagaaataacagaatgttgggagaagataaatctgccttttgaaaccaaatatttaatattttcattaaattgtttcaatacaacttcaggcaaatgccaactggaagaccaagcccagaaattcagaggaaataatcttccaaacaaggaacaccaaggtgatc

SEQ ID NO: 331 - DNA, 61 bp, HOMO SAPIENS
gtagagacagggtcttgttatgttgccagggttggtctccaactcctgagctcaagtgatc

SEQ ID NO: 332 - DNA, 117 bp, HOMO SAPIENS
gatcatacgaggcatgtaataccaagaattgttactttacaatgttcccttaagcaaaattgaatttgctttgaacttttagttatgcacagactgataataaacctctaaacctgc

SEQ ID NO: 333 - DNA, 128 bp, HOMO SAPIENS
gatcaattacctaactgttccttggtctatttatgtataagaatgctttttaaagcacatgtctcattttaaatgacgcacaaactgaagatgttaataaaatttaagagtaatacaaaaaaaaacaa

SEQ ID NO: 334 - DNA, 230 bp, HOMO SAPIENS
gatcttgcctaaagagcagtggaccaaatatgaagaggaaaatttctaccttgaaccgtatctgaaagaggttattcgggaaagaaaagaaagagaagaatgggcaaagaagtaatcatgtagttgaagtctgtggatgcagctgttatgaagatggttaaacttgaaacaaacaattttaagaattatttggtctgaagatgttttactttaaataaatgtctattgtg

SEQ ID NO: 335 - DNA, 296 bp, HOMO SAPIENS
cttttcctttctagccacacaacagtgttatttcaagaaaacaaaacaagtcagagaaagaaaaattacctattacctattggcacatttctgttacgtcaagtctgggcttaagccctgttggtcctgttaagctctttttgttctgtatcaaacaggagtgtcttgagttctttctaatccccatacatcatgtctttgaaatgacaaaagtcccatcctttgttgcccgtgcagtaatctgcttgtggaactctttaagaacatccagctggccaggcctgatgtggatgtat

SEQ ID NO: 336 - DNA, 35 bp, HOMO SAPIENS
gatcagaggcttaaagaaaaggatagattaaaacc

SEQ ID NO: 337 - DNA, 190 bp, HOMO SAPIENS
gatctagttattttaataaaggaactctagaagtttcaagtggccagtgaagtgcgaggaatagagagtaaaggaaataattaataatggcgaccctctgttaagtggtttactttttccattaatataaatttcatttggaaagattaaaaaaacacgtttgattgaacttgataaatcagaaatattt

SEQ ID NO: 338 - DNA, 114 bp, HOMO SAPIENS
gatcttcgtttgtgtgtgtgtatacatgtctagatgtgtttatttgtatgtacacttattgttatatgttgtgtcaaccaaatcagcttataaataaaaaagcactcataaatt

SEQ ID NO: 339 - DNA, 119 bp, HOMO SAPIENS
gatcaagaatttgggtgggagaaaagaaagtgggttatcaagggtgatttgaaattttctgcagcattaaagctggcgcttaataagaataagtaataataaagaaatttctaacattc

SEQ ID NO: 340 - DNA, 50 bp, HOMO SAPIENS
gatcacatcactgtactccagtctgggcaacagagcaagaccttgtctcg

SEQ ID NO: 341 - DNA, 245 bp, HOMO SAPIENS
gtctttgtacaaaattttattaaaggtctttagagagcaacatccagactccagaatacagctgccaaggagaccctgttatgctgtggggactggctggggcatggcaggcggctctggcttcccacccttctgttctgagatgggggtggtgggcagtatctcatctttgggttccacaatgctcacgtggtcaggcaggggcttcttagggccaatcttaccagttgggtcccagggcagca

SEQ ID NO: 342 - DNA, 65 bp, HOMO SAPIENS
gaagtttttagtttattaatgttcttgcgaaaaatccagacttgaactgccgaaggtcccagatc

SEQ ID NO: 343 - DNA, 44 bp, HOMO SAPIENS
cctgttaaaattcaacagctttattgtgaaagaatccagagatc

SEQ ID NO: 344 - DNA, 181 bp, HOMO SAPIENS
gatcatcgggggagggtagtgaagctcaccactgtcctttgttttggagatttttatttttgcataagtagtccatcctatacagatagctgattaactgtattcccctttcccctatggctgctggtgtaaataaactgcatctccccattggtaaacagtaataaaattttaaaaaatg

SEQ ID NO: 345 - DNA, 200 bp, HOMO SAPIENS; identical to SEQ ID NO: 61

SEQ ID NO: 346 - DNA, 215 bp, HOMO SAPIENS
gatctgcaagagctgcatcagtcaaacaccggggataaatctggatttgggttccggcgtcaaggtgaagataatacctaaagaggaacactgtaaaatgccagaagcaggtgaagagcaaccacaagtttaaatgaagacaagctgaaacaacgcaagctggttttatattagatatttgacttaaactatctcaataaagttttgcagctttc

SEQ ID NO: 347 - DNA, 49 bp, HOMO SAPIENS
gatcagatattccttacatttttcaaaactactgcatgtttaaaacttc

SEQ ID NO: 348 - DNA, 205 bp, HOMO SAPIENS
gagatggagttttattcttgttgcccaggctagagtacaatggtgcagtcccagctcaacctccatctcctgagttccaagcagttcccctgtctcagcctcccgagtagctgggattacagacatgcgccaccatgcccaactaatttttttgcatttttagtagagacagggtttcaccatgttttccaggatggtctcgatc

SEQ ID NO: 349 - DNA, 55 bp, HOMO SAPIENS
gatcgcgctattgcactccagcctgggcaacaagagcaaaactccatctcaaaaa

SEQ ID NO: 350 - DNA, 83 bp, HOMO SAPIENS
gatcctgtctgtgctcgtctcttgttgttcatctttaaaagaaacccactttccatttgcgataagagtcttcagtttgttgc

SEQ ID NO: 351 - DNA, 75 bp, HOMO SAPIENS
gatctttctgtgcctcagttttctcatctctgaaatgggacttattctagtatggggttattatgtataaaaaag

SEQ ID NO: 352 - DNA, 78 bp, HOMO SAPIENS
gatcaccgactgaaaatattgttttacaatagttctgtggggctgttttttgttattaaacaaataatttagatggtg

SEQ ID NO: 353 - DNA, 131 bp, HOMO SAPIENS; identical to SEQ ID NO: 24

SEQ ID NO: 354 - DNA, 109 bp, HOMO SAPIENS
gatcatccaagcagttatacctatcaatagctcatttcttttttattgctaagtggtattctctagtatggatgactgtaccacaaataaagatgaagtgaacacttcc

SEQ ID NO: 355 - DNA, 81 bp, HOMO SAPIENS
gaggaagagcaaagctgtcaaataagctactatatcagaagggacataaactgaactagtgccattctgacacacaggatc

SEQ ID NO: 356 - DNA, 114 bp, HOMO SAPIENS
gatctcagttctgcgtttattgtaagttgataaaaacatctggaagaaaatgactaaaactgtttgcatctttgtatgtatttattacttgatgtaataaagcttattttcatt

SEQ ID NO: 357 - DNA, 157 bp, HOMO SAPIENS
ggaggtgctcactcaatgtttattgaacagcgaatacagaaaagacagtccctggagctctatatgtggaaagtagcccttctggacagaaagaatattcgtggtccatgtggtttgagtctgttaagaaggacactaaggcacatggctggtgatc

SEQ ID NO: 358 - DNA, 49 bp, HOMO SAPIENS
gagacagggtctcgccaggttagtctccaacttctaggctcaagtgatc

SEQ ID NO: 359 - DNA, 95 bp, HOMO SAPIENS
aagtgaataataccagtcttcaaagaaaacaaggtgaagacctattgcttcaataatcaagaatgctttgtgtgttttgaggtaggagcatgatc

SEQ ID NO: 360 - DNA, 330 bp, HOMO SAPIENS
gatcgcacgccccccgtggcggcgacgacccattcgaacgtctgccctatcaactttcgatggtagtcgccgtgcctaccatggtgaccatgttcattataatctcaaaggagaaaaaaaccttgtaaaaaaagcaaaaatgacaacagaaaaacaatcttattccgagcattccagtaacttttttgtgtatgtacttagctgtactataagtagttggtttgtatgagatggttaaaaaggccaaagataaaaggtttctttttttttccttttttgtctatgaagttgctgtttattttttttggcctgtttgatgtatgtgtgaaa

SEQ ID NO: 361 - DNA, 64 bp, HOMO SAPIENS; identical to SEQ ID NO: 59

SEQ ID NO: 362 - DNA, 13 bp, HOMO SAPIENS
gggcacaatgatc

SEQ ID NO: 363 - DNA, 51 bp, HOMO SAPIENS
gatcccttctgttggtgtcatgttgtaaacatttctttcaataaactaaag

SEQ ID NO: 364 - DNA, 203 bp, HOMO SAPIENS
gtaagtagtggaggagttggtattttcctgggtatttttagctattacatacctcaattttctaataattgtggtgaacagctgaacttagacttgaacttctgtactcataaacagctgattagaatattgctatctggcttaaattaaaatgcaaaaaatactattagtttcatttcaaaatgtgattgggcatagggatc

SEQ ID NO: 365 - DNA, 56 bp, HOMO SAPIENS
agtttttcaacaaggtttcttttatttatttagcaagaaattactctttattgatc

SEQ ID NO: 366 - DNA, 142 bp, HOMO SAPIENS
gatcaaaaaggtttataaaacagagtaatcttgttggttcaccattgagaccgtgaagatactttgtattgtcctattagtgttatatgaacatacaaatgcatctttgatgtgttgttcttggcaataaattttgaaaagt

SEQ ID NO: 367 - DNA, 238 bp, HOMO SAPIENS
gatctatgtggtgaaaatgcacaggagcttggtagactgcgggggaaagagagagctcctttcgccatgttttaccagtctgctgttataacctcttaggttgtatcctttaatttccagccttttaggttagtttctgtaacagaacaagtgagtctgggatgaagtcctcaaagtacttcaaatggtaattgttttgcttttgtaatagcttaacaaataaacctaggttttctat

SEQ ID NO: 368 - DNA, 116 bp, HOMO SAPIENS; identical to SEQ ID NO: 34

SEQ ID NO: 369 - DNA, 235 bp, HOMO SAPIENS
aaaggcatgataggagtttatttacagacaagacacagcaaccagcacccactcaggctctgaccaggggaggtggtagacaagaccccagatatatataaatatacatataggtcccagcaccatcagcaccagaacgttccccagccaggtccctgccagaaggaccaagagtcaggcaggattgaggacatggaaaggagagggccccagtctttgccaggtatcagtgatc

SEQ ID NO: 370 - DNA, 14 bp, HOMO SAPIENS
gatcagcgctttac

SEQ ID NO: 371 - DNA, 30 bp, HOMO SAPIENS
gatcttcaataaattttttctttaaatttc

SEQ ID NO: 372 - DNA, 60 bp, HOMO SAPIENS
gatcatagatttctgattttatgtaaaattttgcctaatacattaaagcagtcacttttc

SEQ ID NO: 373 - DNA, 132 bp, HOMO SAPIENS
gatcttaaacattgttttgtagtgtatattacttgtccattcctttaaggggagcagcctgcactcttttgtagattacttttgggggatatattttgagaatgatgaaacggaataaaattgtaaaaaact

SEQ ID NO: 374 - DNA, 99 bp, HOMO SAPIENS
gatctggaaagaattcacaggaagatagtgaggactcagaagacaaagatgtgaagaccaagaaggatgattttcactcagcagaggatagtgaagatg

SEQ ID NO: 375 - DNA, 27 bp, HOMO SAPIENS
gatctgacccctattgtggtagaagac

SEQ ID NO: 376 - DNA, 64 bp, HOMO SAPIENS
ggagagatggggtctcaccactgtgttgcctaggctggtcttgaactccagggctcaagtgatc

SEQ ID NO: 377 - DNA, 42 bp, HOMO SAPIENS
aaaaaaaccttactttttgtttaatttgttttgccaaacaaa

SEQ ID NO: 378 - DNA, 148 bp, HOMO SAPIENS; identical to SEQ ID NO: 13

SEQ ID NO: 379 - DNA, 82 bp, HOMO SAPIENS
gaatttacattttacttaaaaaaaggggggtgggggaagtacatacacaaggaggaaaatggggtgaggaacaataaagatc

SEQ ID NO: 380 - DNA, 49 bp, HOMO SAPIENS
gatcctggccaagtcacatcactcctctgtgactcagtttactccttgt

SEQ ID NO: 381 - DNA, 94 bp, HOMO SAPIENS
gatcaaacctattgccactattattaatattctatcagttttaaccagtgtgataaacattaaacaaaaatgacaaagattattactataaggg

SEQ ID NO: 382 - DNA, 32 bp, HOMO SAPIENS
agcttttctggcttgtttaatgctgcgggatc

SEQ ID NO: 383 - DNA, 207 bp, HOMO SAPIENS
gatcctttatatgtgtgttttgggggagctatgataagttttatggcaaacggttggtattgttaactttttattgtcatcaaaagttcataaaagtcctattaatccccatattcttctactgcccttaactctggtatacaccaaaaagaaatctttactttccttgttttatcattataaaaataaagtattttgctagtatgg

SEQ ID NO: 384 - DNA, 247 bp, HOMO SAPIENS
gatcctggactgggtaccttacatcaatggcaagttcaagaaggataattaattacacaaacccttcacagactgctctggtgcctggtggtgctagctccccccacctcagcacctgctgcatctggagcagcccaagcctcaggatggacaagaggaaacccacagctcagcttcaggcttcttatgtttctgaaaacagcttggatattttaatgcacgttgcattaaacctcactgaaacctg

SEQ ID NO: 385 - DNA, 33 bp, HOMO SAPIENS
gatcctaataatcaacttcacgtaattaaaaaa

SEQ ID NO: 386 - DNA, 55 bp, HOMO SAPIENS
gatcgtgaagagaggaatgagaaaaaagctcaaattcaggaaatgaaaaagagag

SEQ ID NO: 387 - DNA, 152 bp, HOMO SAPIENS
aaagaagtaagcctttatttccttgttttgcaaataaaactggctaagttggttgctttttggtgattagtcaaagagaccaaatcccatatcctcgtccgactcctccgactcttccttggcttcaagcacttcagggttgtagatggatc

SEQ ID NO: 388 - DNA, 140 bp, HOMO SAPIENS
acctttaatctctccttttcatttttattttagttcagcttatgcaagattttgtaccttgtgaaagaatttgtttactgaataaaaaatattgcttaagatttaaagcaattaaagcaaaataagttgctgaagggaga

SEQ ID NO: 389 - DNA, 129 bp, HOMO SAPIENS
gatcaatgtaggtattaagaatttaagaatttgctttaaataaaacctttaccacataaaaataagagtattatgttaataaaacatcatgaaaatgaatttcctattttatgtagtaggaaactgtgc

SEQ ID NO: 390 - DNA, 42 bp, HOMO SAPIENS
caggtctctcttcagctcttcctgcacttcaatttgcagatc

SEQ ID NO: 391 - DNA, 131 bp, HOMO SAPIENS
gatccagcccagtttataggttctgttacatctaacttcccaactgtctctagtctggtcacttctccccatctgcccagcaaagatgaggtcgattggacttgtaaaagattaccttttctaaacagttt

SEQ ID NO: 392 - DNA, 114 bp, HOMO SAPIENS
gatctaaataagtgtttcaaggaaagttttcttaagcaaatgtaatattacctcatttgggcatcattactctgttaattctatatcaaaggaaataaacttgctacttgcact

SEQ ID NO: 393 - DNA, 165 bp, HOMO SAPIENS
gatccatctgcctttgtggcagcagccccagctaaggttgaagccaaggaagagtcggaggagtcggacgaggatatgggatttggtctctttgactaatcaccaaaaagcaaccaacttagccagttttatttgcaaaacaaggaaataaaggcttacttcttt

SEQ ID NO: 394 - DNA, 91 bp, HOMO SAPIENS
gatcatcccttgacagtagtttgccttcatctcacctttcatttgtcccaaattcaccttatttaataaagtcccatatgttgtctcactc

SEQ ID NO: 395 - DNA, 106 bp, HOMO SAPIENS
gatctcagaagaagccagcgagcctgtcaacaactggatgtccagaaaaatattcaggttcccagggaagcatggtactggttgaggcttgaagaggagactgaag

SEQ ID NO: 396 - DNA, 119 bp, HOMO SAPIENS
gatcagaatctcttgggttctcttcttcgggtcagtctcctgtttacatttttgttcacagtatttaatttctcacccttggatatagcctaagcattaattaatattacaacaaattc

SEQ ID NO: 397 - DNA, 100 bp, HOMO SAPIENS
gatcaaggtctgcaagggaattcttgtgtgctgctttccatttgacaccgcagttctgttcagccatcagaagagagacaaggaattaaaaatttctttt

SEQ ID NO: 398 - DNA, 89 bp, HOMO SAPIENS; identical to SEQ ID NO: 46

SEQ ID NO: 399 - DNA, 173 bp, HOMO SAPIENS
gatctatttctagaagaactgctgggtcaaaggctctgtacatttacaattgactgattctgttaaatcatcttctaaaaaagcttcactgcattatattcctaccaagaatgtacaagaatatggcctgtttttctatatgtgaaataaagggattggattgtttctctctg

SEQ ID NO: 400 - DNA, 224 bp, HOMO SAPIENS
gatctaaagatttctctatccaatgaatctaacaaagtcaatggaaattgaactctagaattgtctctagaaaacatagcttcttactgaacttgaacatttttacaacattcactggtttttgttttgttagctaataatctataatagttgagtatctctgggaatggggagggaaattatatgtaatagagcttaaaaataaagtgtcaatttccaagggc

SEQ ID NO: 401 - DNA, 60 bp, HOMO SAPIENS
gatctcatttgagcctcacaacgactctggggcaaaggagactgtaaaacaaacaaaaac

SEQ ID NO: 402 - DNA, 48 bp, HOMO SAPIENS
gagatggaatttcgctcttgtcacccaagctggagtgcaatggcgatc

SEQ ID NO: 403 - DNA, 154 bp, HOMO SAPIENS
gatcgattctgtatttttagtagagatggggtttcacccagctactcgggaggctgaggcaggagaatcacttgaaccggggagttggaggttgcagtgagccgagattatgtcactccagcctgggcaacagagtgagactccgtcttaggag

SEQ ID NO: 404 - DNA, 199 bp, HOMO SAPIENS
gatctcttttttttacaccggatgtgacttgtcactggacatgctaatgtcagtcgttactccttatgaagcagacacggacaggtctacagcattacataatagatgtgtcaataaactgtgtgtgtctgtgggtgcatacgtacgtattctagagtagaagttaccataaggttcacacaccagaaacataaaaata

SEQ ID NO: 405 - DNA, 128 bp, HOMO SAPIENS
gatcctaggagagcccccgggacaggacgcgggggcggtagaaagagcacagagaagacaggaggagcgcccgccttccgggtcccagcatcagaggcaggcaaaacacagaaatgagattttggtcc

SEQ ID NO: 406 - DNA, 117 bp, HOMO SAPIENS
gggtttgttatattttattttccaagtaatactgattaaaaatatttacacacctacttttaagtccacagtttagcactcttatttgtatggctttaaaagttatagcacaagatc

SEQ ID NO: 407 - DNA, 103 bp, HOMO SAPIENS
gatctgaaaaacaattcctaaaagattgacttttccagaaaactagctacacaatgcattgcgtctatcatgttaaaacgtgcattagacacaaatacaaaaa

SEQ ID NO: 408 - DNA, 150 bp, HOMO SAPIENS
aacaaactatgtaagagtatggattaagagaagggaatgaggctggaatgtgagtcagaaaatacctgctctaattatagctttgagccaacctctaattctggattcagtgtttttatcagtaaactaaaaggtctatactagatgatc

SEQ ID NO: 409 - DNA, 264 bp, HOMO SAPIENS
gatccattgatttgtagacctgtgcctcacatgacatactaaccaaccaactatgtttctctgtttgcttcacataatacttaaactaactagactacaacataaaaatatacacttaataatagattataaaaataacataatatggtattggttaactgtaaaaaataatagaacttttggaataaatatagttagttggttaatgttagatagaatataagaagattttgtatttggggaataaagagggtggttgggtgg

SEQ ID NO: 410 - DNA, 42 bp, HOMO SAPIENS; identical to SEQ ID NO: 73

SEQ ID NO: 411 - DNA, 130 bp, HOMO SAPIENS; identical to SEQ ID NO: 25

SEQ ID NO: 412 - DNA, 305 bp, HOMO SAPIENS
gatccattgatttgtagacctgtgcctcacatgacatactaaccaaccaactatgtttctctgtttgcttcacataatacttaaactaactagactacaacataaaaatatacacttaataatagattataaaaataacataatatggtattggttaactgtaaaaaataatagaacttttggaataaatatagttagttggttaatgttagatagaatataaaaagattttgtattttaaaataaatatagttagttggttaatgttagatagaatataagaagattttgtatttggggaataa

SEQ ID NO: 413 - DNA, 86 bp, HOMO SAPIENS; identical to SEQ ID NO: 48

SEQ ID NO: 414 - DNA, 182 bp, HOMO SAPIENS
gaaactttaaaatccctcaaaaactgtttattatacaagtgagttttgagtcacgatgggcttatcggtaggatttctggtagcgagcgcgggcaccagggcctccaaactttttggactcgcagcgacgagggtcagctaccagcagggtccggtcatactggatgaggatgtctttgatc

SEQ ID NO: 415 - DNA, 59 bp, HOMO SAPIENS; identical to SEQ ID NO: 63

SEQ ID NO: 416 - DNA, 54 bp, HOMO SAPIENS
caaggaggcaggaaagcaattaagtggtcacctcaacataagggggacatgatc

SEQ ID NO: 417 - DNA, 98 bp, HOMO SAPIENS; identical to SEQ ID NO: 41

SEQ ID NO: 418 - DNA, 64 bp, HOMO SAPIENS
gatcatccttcctggcaaataaattcccgtttctatccaaaagagcaataaaaagttttcagtg

SEQ ID NO: 419 - DNA, 166 bp, HOMO SAPIENS
gatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggttt

SEQ ID NO: 420 - DNA, 49 bp, HOMO SAPIENS; identical to SEQ ID NO: 108

SEQ ID NO: 421 - DNA, 116 bp, HOMO SAPIENS
gatcaccttagtttgattctattttttagcttgcaaaaagtgacttatattccaaagaaattaaaatgttgaaatccaaatcctagaaataaaatgagttaacttcaaayatttca

SEQ ID NO: 422 - DNA, 53 bp, HOMO SAPIENS
gcacttacaaagcagttggggcatttattgacatttaaacaagggaggagatc

SEQ ID NO: 423 - DNA, 97 bp, HOMO SAPIENS
gatcagtttattcagaattgctctataatttaccaattgtatgtatttaacctattcttgtggaaaaaaaaggtctttcattatatctttatttctg

SEQ ID NO: 424 - DNA, 268 bp, HOMO SAPIENS
cattttaattattttattatttttttggtccctaacattcagtctaagggagtaccaaccaaccacccaaccaccctctttattccccaaatacaaaatcttcttatattctatctaacattaaccaactaactatatttattttaaaatacaaaatctttttatattctatctaacattaaccaactaactatatttattccaaaagttctattattttttacagttaaccaataccatattatgttatttttataatctattatta

SEQ ID NO: 425 - DNA, 162 bp, HOMO SAPIENS
gatcctgacactaaggaaatgctgaagcttttggacttcggcagtctgtccaaccttcaggtcactcagcctacagttgggatgaatttcaaaacgcctcggggacctgtttgaattttttctgtagtgctgtattattttcaataaatctgggacaacagc

SEQ ID NO: 426 - DNA, 43 bp, HOMO SAPIENS
gatctttgtacgtaattaaaaatattgtggcaggattaatagy

SEQ ID NO: 427 - DNA, 47 bp, HOMO SAPIENS
gatccagcacacctcctcggtttttttttcaataaaagtttagaaag

SEQ ID NO: 428 - DNA, 138 bp, HOMO SAPIENS; identical to SEQ ID NO: 20

SEQ ID NO: 429 - DNA, 70 bp, HOMO SAPIENS
gatctcttgcctagttaaagggcttcagaaggctgcatataaagccgtcaattatgacaaactaaaaaac

SEQ ID NO: 430 - DNA, 62 bp, HOMO SAPIENS
gatcagaatgttctaaattttgactagttactttttattatgagttaatatagtttagcagy

SEQ ID NO: 431 - DNA, 203 bp, HOMO SAPIENS
gatttgctgtcatcagattcactgtcagataaagattttctttttgtaccatctttttctttgccagctttctgagagttaagaaacgcttcaatcaattctggacaatctaaattttcttcaggttcccaagtattgtcagcatctgtaaatcccttccacttcaggaaatattccactttcccattcactacacgtcgatc

SEQ ID NO: 432 - DNA, 159 bp, HOMO SAPIENS
gatcacctctgagacccaccttgctcataaacaaaatgcccatgttggtcctctgccctggacctgtgacattctggactatttctgtgtttatttgtggccgagtgtaacaaccatataataaatcacctcttccgctgttttagctgaagaattaar

SEQ ID NO: 433 - DNA, 75 bp, HOMO SAPIENS; identical to SEQ ID NO: 50

SEQ ID NO: 434 - DNA, 48 bp, HOMO SAPIENS
gatcaacaaaaatacctgcccacattaagctcctgttctagggaagac

SEQ ID NO: 435 - DNA, 168 bp, HOMO SAPIENS; identical to SEQ ID NO: 4

SEQ ID NO: 436 - DNA, 143 bp, HOMO SAPIENS
gatccactaccggaagaagaaacagctcatgaggctacggaaacaggccgagaagaacgtggagaagaaaattgacaaatacacagaggtcctcaagacccacggactcctggtctgagcccaataaagactgttaattcctc

SEQ ID NO: 437 - DNA, 49 bp, HOMO SAPIENS; identical to SEQ ID NO: 70

SEQ ID NO: 438 - DNA, 49 bp, HOMO SAPIENS
gatcattcttttatagagcatatttgccaataaagcttttggaagccgg

SEQ ID NO: 439 - DNA, 38 bp, HOMO SAPIENS
ggctttaaagtgaatcgtttttatttaaaacagggatc

SEQ ID NO: 440 - DNA, 34 bp, HOMO SAPIENS
cagattaaggcttgttttattttaatggctgatc

SEQ ID NO: 441 - DNA, 17 bp, HOMO SAPIENS
ctgaatttttttagatc

SEQ ID NO: 442 - DNA, 140 bp, HOMO SAPIENS; identical to SEQ ID NO: 16

SEQ ID NO: 443 - DNA, 138 bp, HOMO SAPIENS; identical to SEQ ID NO: 21

SEQ ID NO: 444 - DNA, 126 bp, HOMO SAPIENS
gtttcaagttttaatcaaagcttgtatataagattactttattcctgcatcttctcaatggtttcttccttgtatttgcccttttcctttcctacttggcgagatttggctttccgttcgaggatc

SEQ ID NO: 445 - DNA, 125 bp, HOMO SAPIENS
gatctggattactatgtaaattcacagcagtaagataatataaattttgttgaatgtattaacatcatatggtctgaaaatgtgggtttttatttggcacatttaaataaaatgtttctaactag

SEQ ID NO: 446 - DNA, 108 bp, HOMO SAPIENS
cctttagaattattttattaaatcataaatgtacaacagcttcttaactctacacacgcacttaaatttttttaaaggaaaaacgttatgtcttattacaccatgatc

SEQ ID NO: 447 - DNA, 72 bp, HOMO SAPIENS
gatctaacacaatgtcttaagtataataggtagtctctgtttgtaaaataaatgacttaaatttaaaacatc

SEQ ID NO: 448 - DNA, 58 bp, HOMO SAPIENS; identical to SEQ ID NO: 64

SEQ ID NO: 449 - DNA, 68 bp, HOMO SAPIENS
gtatttttagtagagayggggtttcaccrtgttggtcaggctggtctcgaactsctgacctygtgatc

SEQ ID NO: 450 - DNA, 193 bp, HOMO SAPIENS
gtaattagtagacatggtcttctacccataagctccattacattaaaagaacaaagtgggcttaacatctcacttaagataattcarcattattatcagttttaaaaaaaccaaaaatatcttagaaacaaatcagtctgctttaacaaattgcattcatgctaatgaaattttaatcttctttgtcatgatc

SEQ ID NO: 451 - DNA, 156 bp, HOMO SAPIENS
gatcaaaaagaagatactatcaacgaatttcagaacctaaagtctcttatggaaaacacatttaaatgcaatgacaaggctgatacatcttctttaataataaacaataaattgatttgtaatgraacagttgaagtacctaaggacagcaaatct

SEQ ID NO: 452 - DNA, 167 bp, HOMO SAPIENS
gatcttaatttttctaaaatataaagcatatagtataacatttcacaaaatcaaaatattgtaatgccaggtattgtaactagatgtttaatgatgtgaactgtgtaaattgtttaatagcaatttgtcattactatggaagagacaataaaaatataagcaatatc

SEQ ID NO: 453 - DNA, 161 bp, HOMO SAPIENS
ggcagtttttacatttatttaaacagaaaacgtgcacatgagctgcctactcattttcttcactgcgcagcctggcattggggttggtgactctgatggccagttgggcagctctttccacgatggctttgcggttcttggaggaaacattgtgagcgatc

SEQ ID NO: 454 - DNA, 157 bp, HOMO SAPIENS
gtaatyttcttttattaaaaacatttcctaaaaaatgtgtcttttcctttatgtctgggtgatatatatgactataagctggccttgagcactgtgtggagaggaaaaaggacagctaggagtcttcccaagcatcctatttgtggcttcagagatc

SEQ ID NO: 455 - DNA, 151 bp, HOMO SAPIENS
gatcacttgagcccaagggttagaggctgcagtgagctatgatggtgccaccacactccagactgggcaacagagcatgaccctgtctcaaagaagagggaaaagagcattttctcacctatttttactaacaatgatgctctattttagc

SEQ ID NO: 456 - DNA, 145 bp, HOMO SAPIENS
ccctacaaaataatttattggaacacacagctacagcactctatgtacaagcacattgacgctcctgactatcctcaactaggggacccttttcttcccccttgccttgcggacctcttctatcaaatctttcaggtactggatc

SEQ ID NO: 457 - DNA, 149 bp, HOMO SAPIENS
gatctatcctttacttgaaagcttttgaaaagtggaaaggtcattttgttgcatttccccatttcttgtttttaraagaccaacaaatytcaagccctataaatggcttgtattgaacttttacatttgaattaaagatgttaaacatg

SEQ ID NO: 458 - DNA, 127 bp, HOMO SAPIENS
acatgaaggcaatttattaacagaaaatattttgaggaatcttrttcacagacggcgaccacggcgaccccccttcctgcgagtgctgtcagaggggatgggggtgacatcctcaatccgcccgatc

SEQ ID NO: 459 - DNA, 105 bp, HOMO SAPIENS
gatcccatccatcatactacatcttctcaaatattgagaatattgtcttttaaatatgtttgtatacatrtacacatatatatatacatatataatgaaactgtg

SEQ ID NO: 460 - DNA, 105 bp, HOMO SAPIENS
gatccagctggaaagctgaagtatttcgataaactgaattgagaaatacttcttcaagttatgatgcttgaaagtyctcaataaagttcacggtttcattmccmc

SEQ ID NO: 461 - DNA, 101 bp, HOMO SAPIENS
gatcycctcagaaggcaggagtgctgccctctcccatggtgcccgtgcctctgtgctgtgtatgtgaaccacccatgtgagggaataaacctggcactagg

SEQ ID NO: 462 - DNA, 94 bp, HOMO SAPIENS; identical to SEQ ID NO: 43

SEQ ID NO: 463 - DNA, 86 bp, HOMO SAPIENS
gatcagtgtctctagtcctacccagttttaaagttcatggtaagatttgacctcatctcccgcaaataaatgtattggtgatttgg

SEQ ID NO: 464 - DNA, 85 bp, HOMO SAPIENS
gatccccaaaaagagttcattcaatttcttatgactratgaggaaacagtagataaagctccacctcattctaaaataggtctag

SEQ ID NO: 465 - DNA, 77 bp, HOMO SAPIENS
cttttcgccatcttttgtctttccgtggagctgtcgccatgaaggtcgagctgtgcagttttagcgggtacaagatc

SEQ ID NO: 466 - DNA, 72 bp, HOMO SAPIENS; identical to SEQ ID NO: 55

SEQ ID NO: 467 - DNA, 56 bp, HOMO SAPIENS
gatctcattccagagaaagwggtagaaggtcaaggagcacatcaaaaacaagagac

SEQ ID NO: 468 - DNA, 61 bp, HOMO SAPIENS
ccagctctgcaaagagtttcattgtctgtttatatacagcttgtttgaacagctcaggatc

SEQ ID NO: 469 - DNA, 50 bp, HOMO SAPIENS
gaggtggagtttcactcttgtcgcccaggctggagtgcagtagtatgatc

SEQ ID NO: 470 - DNA, 48 bp, HOMO SAPIENS
gatcgtgccactgaactccaacctgccaatagagcgagactccgtcty

SEQ ID NO: 471 - DNA, 35 bp, HOMO SAPIENS
gatcctactctcttattaaaaagatttttgctgac

SEQ ID NO: 472 - DNA, 106 bp, HOMO SAPIENS; identical to SEQ ID NO: 38

SEQ ID NO: 473 - DNA, 105 bp, HOMO SAPIENS
gatctgggtggtgcttgtgtgggtgtatgtataatacataaaaatttatcaggcagtacacttaagatttatacacttaatagtatctttgttatatctcagcaa

SEQ ID NO: 474 - DNA, 104 bp, HOMO SAPIENS
gatcaaatatcactctcctacttacaggactcaacatactagtcacagccctatactccctctacatatttaccacaacacaatggggctcactcacccaccac

SEQ ID NO: 475 - DNA, 103 bp, HOMO SAPIENS
gatctgaggaggcttcgtgggcttttgggtcctctaactaggactccctcattcctagaaatttaaccttaatgaaatccctaataaaactcagtgctgygtt

SEQ ID NO: 476 - DNA, 103 bp, HOMO SAPIENS
gatctgaatctctgacttattgattatggaacctgtcaagtagttttcaactctcycagtgaggataattaaacatgctcagyctgagtcacctctaagtgtc

SEQ ID NO: 477 - DNA, 101 bp, HOMO SAPIENS
atttatrctctgtggacgtttattaaggctctggcaacactgtggagctggcctgtgtccccgagaagaagagtcagtcacaggggggtctccatctgatc

SEQ ID NO: 478 - DNA, 90 bp, HOMO SAPIENS
gatcctggaggaccctgggccccaggccagctcccatcgctgggggacggtgarcggccatgtgttaatgttacgatgtttttaaaagac

SEQ ID NO: 479 - DNA, 89 bp, HOMO SAPIENS
gagatagggtctcactctgtcacccaggctggaatgcaatggagcaaacatggctcagtgcagccttgacctcctgggctcaaacgatc

SEQ ID NO: 480 - DNA, 89 bp, HOMO SAPIENS
gagacagggtctcactttgtctcctaagctggagtgcagtggcgcagtcacagctcactgctgcctcaacctcctgggcttaagtgatc

SEQ ID NO: 481 - DNA, 88 bp, HOMO SAPIENS
gatctggacagaatcgccggacaggtggcagctgccaacaagaagcattagaacaaaccatgctgggttaataaattgcctcattcgy

SEQ ID NO: 482 - DNA, 88 bp, HOMO SAPIENS
gatcactacaaggaggagaaggactaacaccacagatgacagccccacctccttgagctttatttacctaaatttaaaggtatttctt

SEQ ID NO: 483 - DNA, 84 bp, HOMO SAPIENS
cagkgaagaatgaattctagttaagctctaggaacagaaatggtccttctaaaggagcctaccagggcccctgtgaatacgatc

SEQ ID NO: 484 - DNA, 84 bp, HOMO SAPIENS
gatccccccagcaaggataacattcaaaggagctcacatttatggaatggatgaatcaataaattaattcactttaacaaaaaa

SEQ ID NO: 485 - DNA, 84 bp, HOMO SAPIENS
cttggcaagcattcatttattcacgtaacataagccagacactatgccaggggctggcgatacagaaatgagtaagacatgatc

SEQ ID NO: 486 - DNA, 83 bp, HOMO SAPIENS
ggattccaacaaaacagttttattaaaataaggcacaatrtttgatatggcaaaattcacagaggattggttaatataagatc

SEQ ID NO: 487 - DNA, 82 bp, HOMO SAPIENS
gtaacttttgaattccgtattttttaatggtctatggtctacaaacactcatgtgcaaacacatacacgcagaatagggatc

SEQ ID NO: 488 - DNA, 81 bp, HOMO SAPIENS
rractgtaaatgtattttattttgtaagtgacaatcaaaatatatttgtgctactgattttttcaaatattaaatgggatc

SEQ ID NO: 489 - DNA, 81 bp, HOMO SAPIENS
gatcctagcccagatgattctactggaaattacattgctgtaggaaacatgacccctgttattgaagtgtgggaccttgat

SEQ ID NO: 490 - DNA, 79 bp, HOMO SAPIENS
gatcatgatgtagagtgtggttatgaactttatagttgttttatatgttgctataataaagaagtgttctgcattcgtc

SEQ ID NO: 491 - DNA, 76 bp, HOMO SAPIENS
gctacattttactttattttgttgtaaggaaaaccaattgactaagttgtccccaaaatgttagtgttcactgatc

SEQ ID NO: 492 - DNA, 75 bp, HOMO SAPIENS
ttttgtttttttggaggcgttgagacctgcttgatttattccaagtatttaatacacaatgacgcaactgtgatc

SEQ ID NO: 493 - DNA, 72 bp, HOMO SAPIENS
gatcaaaagtctacataactaatacagctgagctatgtagtatgctatgattaaatttacttatgtaacttt

SEQ ID NO: 494 - DNA, 71 bp, HOMO SAPIENS
gatcctactcctttggagtaaaactagtgcttaccagtttccaattgtatttagcttctggytggaatttg

SEQ ID NO: 495 - DNA, 69 bp, HOMO SAPIENS
attactattattgttcccctaagtggattggctttgtgcttggggctatttgtgtgtatgctgatgatc

SEQ ID NO: 496 - DNA, 68 bp, HOMO SAPIENS; identical to SEQ ID NO: 57

SEQ ID NO: 497 - DNA, 68 bp, HOMO SAPIENS
gatcaacagttactgtgactgagtcggcccattctgtttagaaatatattttaaatgtttagtaattg

SEQ ID NO: 498 - DNA, 67 bp, HOMO SAPIENS
acgyaggactttaatcgttgaacaaacgaacctttaatagcggctgcaccattgggatgtcctgatc

SEQ ID NO: 499 - DNA, 64 bp, HOMO SAPIENS
atggtgaaaagatatatatatatatatattcagaattaggcagctggactcagtttagatgatc

SEQ ID NO: 500 - DNA, 63 bp, HOMO SAPIENS
gaatcatggtagctgcatcacaaaagctccaatcaagagggtggtaaaaagaaggcacagatc

SEQ ID NO: 501 - DNA, 62 bp, HOMO SAPIENS
ggcccagctaattttttatttatttgtagagatggagtcgcgctacgtggcccatgctgatc

SEQ ID NO: 502 - DNA, 62 bp, HOMO SAPIENS
gatctcctgagtccaggaattagagaccggcctaggcaatatagtgagacctcctctctctc

SEQ ID NO: 503 - DNA, 62 bp, HOMO SAPIENS
acagacaaagtgggaggttttatttcttggtctcttcctccttggataaagtcttgatgatc

SEQ ID NO: 504 - DNA, 60 bp, HOMO SAPIENS
atttatatttgagacagagtcttaacactgttgcccaggctggagtgcaatggcgtgatc

SEQ ID NO: 505 - DNA, 58 bp, HOMO SAPIENS
caccactcasctaatttttgtattttcagtagagatgaggtttcaccatcaggtgatc

SEQ ID NO: 506 - DNA, 58 bp, HOMO SAPIENS
aatttttctttagtttattgaaagaaatgtttacaacaygacaccaacagaagggatc

SEQ ID NO: 507 - DNA, 57 bp, HOMO SAPIENS
ataagtgcattattttcatagcacttatcactacctgttgtatattaatcaatgatc

SEQ ID NO: 508 - DNA, 56 bp, HOMO SAPIENS
gatcatttacaggaaatccatgtgttcagtgttgaaaggcataaaatctttttaaa

SEQ ID NO: 509 - DNA, 53 bp, HOMO SAPIENS
gatcttgtttgttgagggagatgggactataataaaattgaatgctgaagttg

SEQ ID NO: 510 - DNA, 52 bp, HOMO SAPIENS
cagcaacatacttctcaatttcttcaggatttaaaatcttgagggattgatc

SEQ ID NO: 511 - DNA, 49 bp, HOMO SAPIENS
gatcatgtcactgcattccagcctgggcaacagaggaagactccgtctc

SEQ ID NO: 512 - DNA, 48 bp, HOMO SAPIENS
gatcacaccactgcactccagcctggcaacagagcgagactccatctc

SEQ ID NO: 513 - DNA, 44 bp, HOMO SAPIENS
ctgtacaaaacctgaaatatttttacttttttaaaactttgatc

SEQ ID NO: 514 - DNA, 41 bp, HOMO SAPIENS; identical to SEQ ID NO: 74

SEQ ID NO: 515 - DNA, 39 bp, HOMO SAPIENS; identical to SEQ ID NO: 75

SEQ ID NO: 516 - DNA, 37 bp, HOMO SAPIENS
ccagttgtaactaatattcctctgtatttattggatc

SEQ ID NO: 517 - DNA, 33 bp, HOMO SAPIENS
cctgctgcatttgctgctttttcagccatgatc

SEQ ID NO: 518 - DNA, 33 bp, HOMO SAPIENS
tttgttttttttgtctttttcactttcccgatc

SEQ ID NO: 519 - DNA, 32 bp, HOMO SAPIENS
ctctttgaaaagtgctttatttcagtaagatc

SEQ ID NO: 520 - DNA, 28 bp, HOMO SAPIENS
ggttgtaggattattggctatcttgtac

SEQ ID NO: 521 - DNA, 290 bp, HOMO SAPIENS
gatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaact

SEQ ID NO: 522 - DNA, 254 bp, HOMO SAPIENS
gagggagctgtaattatttttattggttcgttcagcaatatttcttaacttctggggaaaatacatggtattgccatttcttaattatcacagacactgaactcaaaaattaaaggaagttcgttgcagatgctttttttttagagacaggacaattcttgagtgccgtccttggccttcaccctgattcctgtgagcagatgacttcctctgaagcagcctgattttgtgtatcctttcaccaatcaaggatc

SEQ ID NO: 523 - DNA, 241 bp, HOMO SAPIENS
gatctagtattagattcacagaactgaaaggttaattaagaatgttggttattttcacgggagcgtgtgatatttaacattaatattttatttgacactacagcctgtaatactgctctctttcaaaagcaagttttcagatttttataacagacccattttgttttatgaaacatgttcattacagaaacatgagaaaatagagataagtaaaaagaataaaagtcactattatcctacc

SEQ ID NO: 524 - DNA, 216 bp, HOMO SAPIENS
gatcttttaagtttccttccctacccagtccccattttctggtaaggtttctaggaggtctgttaggtgtacatcctgcagcttattggcttaaaatgtactctccttttatgtggtctctttggggccgattgggagaaagagaaatcaatagtgcaactgttttgatactgaatattgacaagtgtctttttgaaataaagaacyagtccctcc

SEQ ID NO: 525 - DNA, 198 bp, HOMO SAPIENS
gatcagatatacgagaacgccatgattgctgctggacttgttgacgaccctagggccatggtgrgccgcttgaatgagctgcttgtcaaggccctggagcgacactgacagccagggggccagaaggactgacaccacagatgacagccccacctccttgagctttatttacctaaatttaaaggtatttcttaaccc

SEQ ID NO: 526 - DNA, 193 bp, HOMO SAPIENS
caaatgagtttaaatgcattttatttttagacaacctacatgacatgtttttcttaaaaacaatgcctccactccaaataaatcacagtcaaaataaatgaagagctcaagatgacatcagtcccatttgycttaagtcctggtgttgtgtggatgacaagcagaagccagttatgatgacaggtgatagatc

SEQ ID NO: 527 - DNA, 189 bp, HOMO SAPIENS
ccagtcaatgaaagggacactttattgaggctccagggccacggggcctgggcaggaggctgcccttcaaggaaagaaccaccttatttgaccttcttcttgggacgcaggttgttggtgtgaccacacttcttcttgcggcagttgacagcacgagggtgaaggcgagcatagcacttgcggcagatc

SEQ ID NO: 528 - DNA, 183 bp, HOMO SAPIENS
gttcttttaactcatctgtatttgttacaactttaagcagaatgtgactcgagcactacatttccatccacaagactgggtctgagttatttttgaacagctttacgatatgcttaggtaggcttttaactttgctcctccaaacaatacttttctttggaaaacaagccctgtggagagatc

SEQ ID NO: 529 - DNA, 182 bp, HOMO SAPIENS
gacaattgttatttagtttttatttcatartcataaacttaactctgcaatccagctaggcatgggagggaacaaggaaaacatggaacccaaagggaactgcagcgagagcacaaagattctaggatactgcgagcaaatggggtggaggggtgctctcctgagctacagaaggaatgatc

SEQ ID NO: 530 - DNA, 178 bp, HOMO SAPIENS
gatcccttttttattttgtggttttttaatatagtataaaaatccttttaaaaaaacaacaatctgtgtgcctctctggttgtttctcttttttattattactcctgagttgatgacattttttgttagatttcatggtaattctcaagtgcttcaatgatgcagcatttcttgcact

SEQ ID NO: 531 - DNA, 167 bp, HOMO SAPIENS
caagtatcacaatgtttattgatagatacaagtatataaaatcagggcatgaacatgacttgataaattaagtagacttaatttcaatactataataggagggaccaattcaaattctcaccatttgtttcacacccacaaaaaccacttcaagggcattaacgatc

SEQ ID NO: 532 - DNA, 166 bp, HOMO SAPIENS
ggaaagytgaatattttattatttacacatataaagtgagaatgaaaattgggcatggggcaagggcaggaagatgactccagttcagtcggtgatgatgagctcgtccaccccccagtcttcatagctcccatctggcaggtaacggcgaatgatgatggggatc

SEQ ID NO: 533 - DNA, 165 bp, HOMO SAPIENS
gatctttccattggaaataactggaagtgaagaggttttgttgcttgtacagtgtcagatgaggaacaccactatcttaattttgcgatacactgcatttgctggtgctatttttatacagtgaagcaacagctttgcagcaaaataataaaatacttcttcgtt

SEQ ID NO: 534 - DNA, 164 bp, HOMO SAPIENS
gatcaccaaaaaagggggaaaggagcccataaaattaaactacctcycccctcaaaaataaaataaaataaaataaaataaaaacacccacacccctgcagctaacctgacaactaccttcattcacagtgctttatacttaaaccaggatggaggaaatgaat

SEQ ID NO: 535 - DNA, 163 bp, HOMO SAPIENS; identical to SEQ ID NO: 6

SEQ ID NO: 536 - DNA, 163 bp, HOMO SAPIENS
gatcatctttagattattcttcagacagtgagcagtcaagtgttcaggccacacagtcagcccaggaaaaagagaagcagggccaaatggaaagaacacataataaacaaraaaaaaacagarkygrakaaaaatccaagtctgaacgggaatgccctcattc

SEQ ID NO: 537 - DNA, 162 bp, HOMO SAPIENS
cttagtgaagattgcattttaatgctctgacaactcccatttcttcattgtcagaataacctggatagaggccaaagcttaaaacaatttgtacatatgtaaaaagaaaagaaaaatgagaaacctaaattgccgtaagagttcagggcagcagagcagatc

SEQ ID NO: 538 - DNA, 161 bp, HOMO SAPIENS
gatccagattcaatcgactgggtttatgtcccttcacatagtttttaaggttatttatttaaatgtctaatgtattttattgtaacagacattgttttgccaacattgcctatttcagtggcacgtcatctagttttaaaaaaataaaacattttaaatgg

SEQ ID NO: 539 - DNA, 153 bp, HOMO SAPIENS
agtggtataactatatttattgtgcctgagaggcaaggtgagggaaaaatctcaacagaagcaagtttggggaaaatctggagtccccagtaaaaagcaggaaggtctctgctgtactcatcacagaatggaagagagggctctcaatagatc

SEQ ID NO: 540 - DNA, 150 bp, HOMO SAPIENS
cccttgttcagacattttatttgaatttatgacagtgatggggatttgactgagatgccttatggagaagtaccccaccctctatgargacagaatcactcyctgccattcattctgcctgatgctaacaacacgcagctgatttaggga

SEQ ID NO: 541 - DNA, 146 bp, HOMO SAPIENS
gatctgacccaccagtttgtacatcacgtcctgcatgtcccacaccattttttcatgaccttgtaatatactggtctctgtgctatagtggaatctttggttttgcatcatagtaaaataaaataaacccatcacatttggaacac

SEQ ID NO: 542 - DNA, 140 bp, HOMO SAPIENS
gatttaaaagattttattttctttatgcaggtaggcagttagaaatttcaaagtctaacaatgacattcttgaagtgggcacagccttttaaactcaggctatgtatacagtaaccttgtggaactggttcagccagatc

SEQ ID NO: 543 - DNA, 137 bp, HOMO SAPIENS
gatcaagttcccgctgccccaccgggtyctgcgccgtcagcacaagccacgcttcaccaccaagaggcccaacaccttcttctaggtgcagggccctcgtccgggtgtgccccaaataaactcaggaacgccccggt

SEQ ID NO: 544 - DNA, 135 bp, HOMO SAPIENS
gatctctccttcagtctgctctgtttaattctgctgtctgctcttctctaatgctgcgtccctaattgtacacagtttagtgatatctaggagtataaagttgtcgcccatcaataaaaatcacaaagttggttt

SEQ ID NO: 545 - DNA, 132 bp, HOMO SAPIENS
gaggctattatctcctttattcaaaaaataaatatttcactttgattcaacatatttcaaatcacattaagcacagagaagaaatacagtccctttggagattgtgaacacgtgttggaaagaggcatgatc

SEQ ID NO: 546 - DNA, 132 bp, HOMO SAPIENS
rgtcacacaattttaattgtgaattatagtgaatggtaaacagtaaaagagtaattacatgaaaagctccatattttgcatgtttcaactgttcttcattgtctttacttgtaaggtgttgaaggtgggatc

SEQ ID NO: 547 - DNA, 125 bp, HOMO SAPIENS
gatcacttytgaataagcagtttgcctttataaaaacttgctgcctgactaaagattaacaggttatagtttaaatttgtaattaattctaccatcttgcaataaagtgacaattgaatraaaaa

SEQ ID NO: 548 - DNA, 121 bp, HOMO SAPIENS
gatcgcccaataaacattcccttggatgtagtctgaggccccttaactcatctgttatcctgctagctgtagaaatgtatcctgataaacattaaacactgtaatcttaaaaaaaaaaaaa

SEQ ID NO: 549 - DNA, 120 bp, HOMO SAPIENS
gatccaagtgtttaacaattggggctgttaagtcygaccatacatcactgtgatagaatgtgggctttttcaagggtgaagatacaagtcttaaccacagtgtaacttacagtttccttt

SEQ ID NO: 550 - DNA, 120 bp, HOMO SAPIENS
ggymtaaatagactttattgaagtcagtgcctctgtactgasacagaagattgtgtctacataagcacaagttgtaacatttcacaacttctaaaaggaatgtcaacaattacaacgatc

SEQ ID NO: 551 - DNA, 117 bp, HOMO SAPIENS
gatccacttctgtgattaagtaaatggatgtctcgtgatgcgtctacagttatttattgttacatccttttccagacactgtagatgctataataaaaatagctgtttggtaaccat

SEQ ID NO: 552 - DNA, 114 bp, HOMO SAPIENS
ttttcttttttttcatgataaggtggatgtttatttaaaacaaaaataagaagagacatggcctatcatcatgaagattcataggaagagttttcctttgttaacgtggagatc

SEQ ID NO: 553 - DNA, 113 bp, HOMO SAPIENS
gatctagtaggcacgtctgtcaacaggacacatgcctcctctgactataacctcttaatagttgtgtataatgaaaactgtaaacttttttaaataaacgtgtatataccttg

SEQ ID NO: 554 - DNA, 111 bp, HOMO SAPIENS
gatcatttagttctatctatttagaaatatgtaaaactggatttttttttaagtaatatgtgaycaaagttaattttgtcccaaaggtctaaataaagagcagtttcccat

SEQ ID NO: 555 - DNA, 108 bp, HOMO SAPIENS
gactcgtatttaatttatttagaatcttacaaaaacaaaaaacaaaacaaaaaccaccacaacagaaaaaaaactaaatacagaatttgttacgcttcacggttgatc

SEQ ID NO: 556 - DNA, 106 bp, HOMO SAPIENS
gaaggtttttaaaatttattttttaaatgtataatgggcacataattgtacatatatatgggatacaatgtggtgtttcaatgcatgtacacacagtataatgatc

SEQ ID NO: 557 - DNA, 122 bp, HOMO SAPIENS
gatcgagcttctcctctgagttctaacaaaatggtgctttgagggtcagcctttaggaaggtgcagctttgttgtcctttgagctttctgttatgtgcctatcctaataaactcttaaacac

SEQ ID NO: 558 - DNA, 39 bp, HOMO SAPIENS
gatccttgatatgcttagcctgcctcttcctcccctctc

SEQ ID NO: 559 - DNA, 151 bp, HOMO SAPIENS
ggttttgttttttgttttttgtttttttttaatgggagccaaaatgtttagagaaagcttctgggtgtgcctttcagattttgaacaagctgccttgtctaaacatcaatttctactactctgtagccttaaaatgtgctctatttagatc

SEQ ID NO: 560 - DNA, 77 bp, HOMO SAPIENS
gatcaggtgtgtagtttagaaggctattataagggtgtcaataaggctgaattatataacatatattctttccatag

SEQ ID NO: 561 - DNA, 188 bp, HOMO SAPIENS
acaaaaagctacaagtttattaacataatgtattccaatatgtatcaaagtttcaagcaggtcattttgaacctcaaataagaaatcacaatatttagtattaattaaaaaggaaaaaaaaggtggtagttgtcattcgtgggatttagtccaaatgaaaatgtagaaaacatttattttggtggatc

SEQ ID NO: 562 - DNA, 49 bp, HOMO SAPIENS
gatcccgccactgcactgcagcctaggtgacagagcaagactccgtctc

SEQ ID NO: 563 - DNA, 76 bp, HOMO SAPIENS
gagcttctggtaagcattatgtttgttaatacatatttattagagtgatgttttaagttaataaagtattaagagc

SEQ ID NO: 564 - DNA, 334 bp, HOMO SAPIENS
gatcttggccaacgtgaaataaaattactcattcaaaactctgcctaaggtgattttgtagttcttaacagttctccagagcatcttgaacaggaatattaagataaatgtgaatctgcaatggctgaaaagagttgtgagcttttttattcatgataaaaccttataggaatagtataaaaaatccctgtggaaagctactagtacattggccagcgctgggtgatacagattctgataaaaacataaatgtattagttcatctccatgtagtaaaaagtatacttatacaatgttttgtacttgtatttcatgaaattaaaacagtgatgcc

SEQ ID NO: 565 - DNA, 67 bp, HOMO SAPIENS
gtttattgctaaatgtattatgatatataaacaggcaattgaatttagctttttatctactatgatc

SEQ ID NO: 566 - DNA, 52 bp, HOMO SAPIENS
gccaaagacaaactagagcaatgcctatgtaagaacaaggactctcaagatc

SEQ ID NO: 567 - DNA, 41 bp, HOMO SAPIENS
ggaacacaggcttgctctgtcagccaggttgtggtgtgatc

SEQ ID NO: 568 - DNA, 48 bp, HOMO SAPIENS
gatctggtggatgtcaatacccctgatttaatggcacctgtgtctgcc

SEQ ID NO: 569 - DNA, 124 bp, HOMO SAPIENS
gatcctgagctgcacttacctgtgagagtcttcaaacttttaaaccttgccagtcaggacttttgctattgcaaatagaaaacccaactcaacctgcttaagcagaaaataaatttattgattc

SEQ ID NO: 570 - DNA, 58 bp, HOMO SAPIENS
gttctaagagtgccctccggagggagaccctctgttcaagctcgcgaagctctcgatc

SEQ ID NO: 571 - DNA, 42 bp, HOMO SAPIENS
ggttaatgaaatgccagggtgaaagggatggccagttggatc

SEQ ID NO: 572 - DNA, 140 bp, HOMO SAPIENS
gtatcatcctgaaacacactggttgctcagcaaaaatagtatctcttacctccttattttattcccattctttctccttccctccctttttctctctctaccttcctgacttttctttcgtgtttcctttctgattgatc

SEQ ID NO: 573 - DNA, 163 bp, HOMO SAPIENS
ctgaaaacaagttttatttaaataagggtttaaatacattacacataacattaaaactgaaggggaaaaaaaaaccaaaaaccagtttgttacttcacatggcattgggcagctgctgctattaagttgcaagctctacagctagctacatgactgatggatc

SEQ ID NO: 574 - DNA, 105 bp, HOMO SAPIENS
ttgttttgttttctgacatattctgacaaagagcagcaaaccactgctgtgtggcattcttggagtgtgctgtgaatgtgctttttaagaaattaaaaagagatc

SEQ ID NO: 575 - DNA, 30 bp, HOMO SAPIENS
aggggcagcagccacaaaggcagatggatc

SEQ ID NO: 576 - DNA, 56 bp, HOMO SAPIENS
gatcttatgccagaaagtaagaaaaagaaagaagatgacaaaaaaaaaaaaaagaa

SEQ ID NO: 577 - DNA, 202 bp, HOMO SAPIENS
gatctcagaacaatcagatgcaaagctgaaagagattgtaacaaatttcttggctggatttgaagcttaaactcctgtggattcacatcaaataccagttcagttttgtcattgttctagtaaattagttccatttgtaaaagggttactctcatactccttatgtacagaaatcacatgaaaaataaaggttccataatgc

SEQ ID NO: 578 - DNA, 73 bp, HOMO SAPIENS
gatcttggttttccatgaatacagcatgtataataaaaattttaagaaataaatgttattctactttattaac

SEQ ID NO: 579 - DNA, 117 bp, HOMO SAPIENS; identical to SEQ ID NO: 33

SEQ ID NO: 580 - DNA, 124 bp, HOMO SAPIENS
gatctaaagtgaccttgatggacagtggaagaaatcacaacatggaattcctcgaataacaatttattgactttaaataattttgtctaatgctacatatacacaattaaaaaacctttacacc

SEQ ID NO: 581 - DNA, 63 bp, HOMO SAPIENS
gatcctcccccccagtgctttgtagtctctcctatgtcataataaagctacattttctctgag

SEQ ID NO: 582 - DNA, 92 bp, HOMO SAPIENS
gatcccttgcagttctgtacctgttgttctacgagtcaagagcagtgaccagtgacgggggaaacacagggttaataaatgccatactccct

SEQ ID NO: 583 - DNA, 37 bp, HOMO SAPIENS
gtttcttttcttcttctcttctagtttttatctgatc

SEQ ID NO: 584 - DNA, 41 bp, HOMO SAPIENS
gatcaaggctcatgtagcctcactggagggcattgccccgg

SEQ ID NO: 585 - DNA, 54 bp, HOMO SAPIENS
tctgattattaatgtccgtggtaataataaatgactcaatattagctttgtgcc

SEQ ID NO: 586 - DNA, 48 bp, HOMO SAPIENS
gagacagattctcgctctgtcgccgggctggagtgcagtggcgcgatc

SEQ ID NO: 587 - DNA, 97 bp, HOMO SAPIENS
gcttgcacttttttgtctgaagatttatccttcgctgctgcctttttcggcttcgcttccacttttgcaggaggtttagctgacaaccgcgccgatc

SEQ ID NO: 588 - DNA, 47 bp, HOMO SAPIENS
gatcctaaatcatgacttacctgctaataaaaactcattggaaaagt

SEQ ID NO: 589 - DNA, 91 bp, HOMO SAPIENS
tcgccagtgggcacttttttttttccaccctggctccttcagacacgtgcttgacgctgagcaagttcaataaagattcttggaagttttg

SEQ ID NO: 590 - DNA, 33 bp, HOMO SAPIENS
gatcattaaaaatggctgaccctaacaatatgt

SEQ ID NO: 591 - DNA, 16 bp, HOMO SAPIENS
gatccaggcttctgcc

SEQ ID NO: 592 - DNA, 52 bp, HOMO SAPIENS
gcatttttagtagagacagggtttcaccatgttagccaggatggtctcgatc

SEQ ID NO: 593 - DNA, 70 bp, HOMO SAPIENS
gatctcattcagtttgaggagcgccctcaagagccttccgaaattatgattttaaaccataaaacaaaaa

SEQ ID NO: 594 - DNA, 69 bp, HOMO SAPIENS
gtattttcagtgaagatggggtttcaccatgttgccaggctggtcttgaactcctggcctcaagtgatc

SEQ ID NO: 595 - DNA, 60 bp, HOMO SAPIENS
gatcgcgccactgcactcaagcctgggcaacacctgggtgacagagcaagaccccatctc

SEQ ID NO: 596 - DNA, 35 bp, HOMO SAPIENS
gacaaatcttattttattcagatagcagtctgatc

SEQ ID NO: 597 - DNA, 77 bp, HOMO SAPIENS
aagagacggggtcttggccgggcgcagtggctcaggcctgtaatcccagcactttgggaggccgaggcgggtggatc

SEQ ID NO: 598 - DNA, 66 bp, HOMO SAPIENS
caaccttggatttagcggctgagtacttcctcttgtacatggcctttctggaatacatggcagatc

SEQ ID NO: 599 - DNA, 187 bp, HOMO SAPIENS
agatttttaattttcatgggtatatagtaagggtaggtgttattattgtccctcctatcttacagatgttaaggtccctaaagtgacacaggcagtaggtggcagacctaggactgaaactcaagcagattggttccagagtccatggcttaaccctcctactgtttcccagatggaagggatgatc

SEQ ID NO: 600 - DNA, 50 bp, HOMO SAPIENS
gatcgtgccactgcattccagcctaggcaacaagagggcaactccatctc

SEQ ID NO: 601 - DNA, 29 bp, HOMO SAPIENS
gatctgctttctgccatcctactatttct

SEQ ID NO: 602 - DNA, 127 bp, HOMO SAPIENS
gatcgaactatgaaaagtgttaatgaccagccatctggaaatcttccatttttaaaacctgatgatattcaatactttgataaactattggttgatgttgatgaatcaacacttagtccagaagagc

SEQ ID NO: 603 - DNA, 218 bp, HOMO SAPIENS
gatctgatttgtaacccactgagaggacagagagaaataagtgccctctcccaccctcttcctactggtctctctatgcctctctacagtctcgtctcttttaccctggcccctctcccttgggctctgatgaaaaattgctgactgtagctttggaagtttagctctgagaaccgtagatgatttcagttctaggaaaataaaacccgttgattacc

SEQ ID NO: 604 - DNA, 48 bp, HOMO SAPIENS
gactagaccttccttaaacttttagaaataaaatgaaggcttcactgg

SEQ ID NO: 605 - DNA, 94 bp, HOMO SAPIENS
acattagcttcataaactcgatttataacatcattccccaactcacacataagctttaaaagttctggctcccaggtgtctaaagttaaagatc

SEQ ID NO: 606 - DNA, 117 bp, HOMO SAPIENS
acctatccctggagcaagtaataggaagagaatgggcaaactggttgcacgagagataagagaatggagctgggagcaacacatgaacttgcgttataacattctgctgtccagatc

SEQ ID NO: 607 - DNA, 89 bp, HOMO SAPIENS
gatcgcttgagctcaggcattaaagaccagcctgggtaatcatagtgagacccccgtatcaataaaaaatcataataaaagttgaaact

SEQ ID NO: 608 - DNA, 222 bp, HOMO SAPIENS
gtattttctgggtttattcccccttgttccaagctctaagcctcagcttactccacctccatagcctgcacagtttcctccactagctccagggttagtggcttcactgtctgggtcaggacagctgtggttccaggcacaaagtggtagcggccagacctcttcacgggctctgtgggtgagctcagtgtccgcagcagcttggcgccagtctttgtgatc

SEQ ID NO: 609 - DNA, 112 bp, HOMO SAPIENS
catcccactttttccaaccaatccacctataccccagatgtgtgaggccatcctagaccatccagccccaaatgaatcagcttggactagaagaatttccaagccaactcac

SEQ ID NO: 610 - DNA, 54 bp, HOMO SAPIENS
tgcgaaataggctcaaagattctttcttgtggagaagttattcatgtgaagatc

SEQ ID NO: 611 - DNA, 60 bp, HOMO SAPIENS
aaaaattccatcaagcatttacatagtaaatttctataatttcacaaaagattcttgatc

SEQ ID NO: 612 - DNA, 85 bp, HOMO SAPIENS
atatatatatacttttattattcacccgttaactccattatatgccaatcacgagctagttttcagcagttacattcccttgatc

SEQ ID NO: 613 - DNA, 129 bp, HOMO SAPIENS
atatggatttgaaaaacagaaaatatactttatgttctgaaatttgtatttaagtataaaatgtgaatcatcttgtctaaatagcttacagcatagttggcttaaatgaaaataaaatgatatgcttat

SEQ ID NO: 614 - DNA, 125 bp, HOMO SAPIENS
gatcacagtgtcagaggcgcgtcctctttcttggggaagttgaggagtgcccttcagagccagtagtaggcaggggtgggtaggcaccctccttcctgtttttatctaataaaatgctaacctgc

SEQ ID NO: 615 - DNA, 57 bp, HOMO SAPIENS
gatcctttgaacctaggagtttgagaccagcctgggcgatatagtgaggcccatctc

SEQ ID NO: 616 - DNA, 87 bp, HOMO SAPIENS
gatcttttttatttttatacacatgacaagattttacatcaagaatagtcagttaaatagtacaaatttacattcatgaggaatgtt

SEQ ID NO: 617 - DNA, 80 bp, HOMO SAPIENS
acttttgctttatcttcaatgaacttctttccaaatgccccaaatctggcaataagttattatttttaagagaagggatc

SEQ ID NO: 618 - DNA, 93 bp, HOMO SAPIENS
gatcacagaccaacaagtaagccaacaaaaaattaaccagcacatttagcttctcttttttctttttaaataaatattgaatatgattctgtt

SEQ ID NO: 619 - DNA, 159 bp, HOMO SAPIENS
gatccaagggctgtggtaaacgggagagcttgtgtttttgaagtggaaaaaaacccaagagtttgtacagacatcctgtcttcccagagaaggtggacactcttgggctcattgtaaagtgcctgctgcatcaataaagctcttggcttattagtctat

SEQ ID NO: 620 - DNA, 55 bp, HOMO SAPIENS
aatagtgttggacttgttctgacatgcagttaagttactcgggttcagtttgatc

SEQ ID NO: 621 - DNA, 86 bp, HOMO SAPIENS
gaagccagctatttaattaggttcttaaaacatttaaaacaccaatttgggaggataaattccattcgtcagagcaaacacagatc

SEQ ID NO: 622 - DNA, 85 bp, HOMO SAPIENS
gatccaataacttgaccaacggaacaagttaccctagggataacagcgcaatcctattctagagtccatatcaacaatagggttt

SEQ ID NO: 623 - DNA, 80 bp, HOMO SAPIENS
  misc_feature (77)..(77): n = a, g, c or t
gatcctgccactgcattccagcctgggcaactgggtgtctccagtgggagacactgtctcaaatttaaaaaaaaaanaaa

SEQ ID NO: 624 - DNA, 110 bp, HOMO SAPIENS; identical to SEQ ID NO: 36

SEQ ID NO: 625 - DNA, 49 bp, HOMO SAPIENS
gatcacaccactgcactacagcctgggctacagagtgagactttgtctc

SEQ ID NO: 626 - DNA, 144 bp, HOMO SAPIENS
tttcactaagctgtttgccttccaaagtgtttacaccttaagccttaacatgtatcttcattcagaaaacagttatattgtcataccatagtaggaagaaaaacctttatttggaatatacactactgtaaatttgtacagatc

SEQ ID NO: 627 - DNA, 101 bp, HOMO SAPIENS
agcaataaggtcttatcatgcctaggctggtctcgaactcctggactcatgcaatcctaatgccttggccttccagtgctgggattacaggcgtgaggatc

SEQ ID NO: 628 - DNA, 76 bp, HOMO SAPIENS
aagattaagcccgatttgcaacttttattgaaataaatgtcatctactaaaaacaaggttaatttataactggatc

SEQ ID NO: 629 - DNA, 45 bp, HOMO SAPIENS
gatcagaattctgagaccactaacaatgacacataaaaaaacagg

SEQ ID NO: 630 - DNA, 41 bp, HOMO SAPIENS
gatctttatgcaaagcagggtcgaggaagccagtttacatc

SEQ ID NO: 631 - DNA, 47 bp, HOMO SAPIENS
gagaccgtctcacactgtcggccaggctggaatgcaatggcgtgatc

SEQ ID NO: 632 - DNA, 49 bp, HOMO SAPIENS
gaatgaagaaatctggcttttaatgtccgtacatcaagactatgtgatc

SEQ ID NO: 633 - DNA, 51 bp, HOMO SAPIENS
gatcatggccactatacttgcaccagggcgacagagcaagaagactgtctc

SEQ ID NO: 634 - DNA, 163 bp, HOMO SAPIENS
gatcagacagaataatattttctagttattatgtgtaagatgagttgctatttttctgatgctcattctgatacaactatttttcgtgtcaaatatctactgtgcccaaatgtactcaatttaaatcattactctgtaaaataaataagcagatgattcttat

SEQ ID NO: 635 - DNA, 231 bp, HOMO SAPIENS
gatcattgatttctgtacagtcagaacagtacttgggtttgcaacagctttctgagaaaagctaggtgtttaatagtttaactgaaagtttaactatttaaaagactaaatgcacattttatggtatctgatattttaaaaagtaatgtttgatgctcctttttatgagttaaattattttatacgagttggtaatttttgctttttaataaagtggaagcttgctttttt

SEQ ID NO: 636 - DNA, 99 bp, HOMO SAPIENS
gatccagagtggaagaacaccaaagcaaagttttctccaaaataacacacacggatttctgaaaaacagcagttactttcactgaatgtgacaaacttc

SEQ ID NO: 637 - DNA, 117 bp, HOMO SAPIENS; identical to SEQ ID NO: 31

SEQ ID NO: 638 - DNA, 206 bp, HOMO SAPIENS
  misc_feature (5)..(5): n = a, g, c or t
ctggncttaaggcaggttagtttaattttatagaagaaatatttttcaaacagtacaaatgacacaattagagagaaaagagtgacaaaaaacaaagagatgaatactgattataacttattcccacaacagaaggctacttatttctacaattatcaatcaaagcctgacaattcagtgattagatgaagtgaaagccaaattcc

SEQ ID NO: 639 - DNA, 144 bp, HOMO SAPIENS
ttttcttttttcttcttctttgtcttccttttcttcagcctcatcatcgcttacttctttatcacgttccttctccacaaaaagagtaatgggatatccaataaactgagaatgtttcttcacaatctcctttattcttcgttc

SEQ ID NO: 640 - DNA, 63 bp, HOMO SAPIENS
gagtagaggtggaggttttgctatgttgcctatctggtcgtgaactcctggcctcaagggatc

SEQ ID NO: 641 - DNA, 40 bp, HOMO SAPIENS
gatctcaactatgaaataactttaatatttatacattgaa

SEQ ID NO: 642 - DNA, 113 bp, HOMO SAPIENS
atttccacagtccagtaatttattttaaatttgagtaatttcaaattccacaaacaaaactgaagaacagcaatattttgtttgaatttctctcttctgtacactcagtgatc

SEQ ID NO: 643 - DNA, 76 bp, HOMO SAPIENS
cctccaaacagatttattgaatacagcaaaattctatatacaaagtgacctggacctgctgcttcaaaacatgatc

SEQ ID NO: 644 - DNA, 179 bp, HOMO SAPIENS
gatcgtatttcagttttttgggtcaatatatgaacaatgtgtgaaccagaacgtatggtatttgacatcaaagctatgtcttggcatatgtttatgttgatatacttgaattcatttaaaatggaatgttaattacatcgacttcttactataaaaaataaaaagttattgtccagtgc

SEQ ID NO: 645 - DNA, 190 bp, HOMO SAPIENS
gatcacttgaggtcaggatgggaaaattttttaaaagacatttattaaaaaaaaatgtctatgccttatcaaagtttataactcaagaattaatgagataaacagtggacaaaaagtgttggcagataacatgcataattttaattaaaagttaagcagtgaaaaataaacatctttttttcttaaagag

SEQ ID NO: 646 - DNA, 116 bp, HOMO SAPIENS
accagtgttaaaaccagaaattggtattttttaaaatggtttgcttcacatatttaagagttttctttctgtcatttatgaagaataaggaatgtattatggaaaagagggtgatc

SEQ ID NO: 647 - DNA, 151 bp, HOMO SAPIENS
cagtatgagatggaaggttttattagactaggagatttgtgggaggtatgagcgctgggccaaggtggtacgggccatgctcaaggcaccctctcgggtgcggtgaccgccaatgaagccgcttgcatggacgaagatgcagccagggatc

SEQ ID NO: 648 - DNA, 41 bp, HOMO SAPIENS
atctgtaataattgccagtttattcctctggctaagagatc

SEQ ID NO: 649 - DNA, 47 bp, HOMO SAPIENS
ctcaggaaaatttagcatctgttgagcagcaggaacctgaggtgatc

SEQ ID NO: 650 - DNA, 123 bp, HOMO SAPIENS
gatctttttcagaagtgattattagttgggcattaaacctggtttcattctgagtctaaagaatcctccgggccactctgcctatggggtagccctgctctgtctatggagaagctactctag

SEQ ID NO: 651 - DNA, 120 bp, HOMO SAPIENS
gagcaaagcgtacaaaggttccaagggacaggaccaagaacgaggggctgagacatttacaacagcaggcattttctcttcctcttcttcacgggaggcgggcagaggactgctcggatc

SEQ ID NO: 652 - DNA, 87 bp, HOMO SAPIENS
gatagtgactagctctgtcacctaggctagagtgcagtggtgcaaacatgactcactgcagccttgacctcatggactcaagtgatc

SEQ ID NO: 653 - DNA, 144 bp, HOMO SAPIENS
gatcaaaccacatatgttattgggagaaacgatttctgttgacgtagatattgaaagaataatgaaggcagaagagaaaaacgaagtgtggaatttggggttgtcctgtgtaaattacacaataaagcaaaagtcagttattgc

SEQ ID NO: 654 - DNA, 63 bp, HOMO SAPIENS
aagagtcggggtcttgctatggggtgtagtaggttggtcttgaactcctatgttcaagcgatc

SEQ ID NO: 655 - DNA, 32 bp, HOMO SAPIENS
gatctccactctcaggaacttcaggaggggca

SEQ ID NO: 656 - DNA, 151 bp, HOMO SAPIENS
gcaagttgttttatttacaatgccaacttttaaaaggtcactaaagttaactggacaataaactaggcagacattgctttacaaaaaagagggaaagtcatttttgatattgaaggtcttgcgaatctcctcaggagttttccccttgatc

SEQ ID NO: 657 - DNA, 97 bp, HOMO SAPIENS
ggtttgtttttaaagaaactgcatacatcatcagagacaaacaataatttaaataccatgcagtttctgtatgtacaaaacctaggccacagagatc

SEQ ID NO: 658 - DNA, 70 bp, HOMO SAPIENS
gttttgttaaagaaatgagtgtctcattatgttgcccaggctggatttgaactcctgagctcaagggatc

SEQ ID NO: 659 - DNA, 36 bp, HOMO SAPIENS
gatcttgactataataaagcttccctactgcaaatc

SEQ ID NO: 660 - DNA, 60 bp, HOMO SAPIENS
gatcccttttcttttgatggaccagaaattatgggttgtacagggtgccagatagattgg

SEQ ID NO: 661 - DNA, 74 bp, HOMO SAPIENS
gatctgcctcagtgaatcagacaagacagccgtgctcaccttaataagagaagagataattactaaagagattg

SEQ ID NO: 662 - DNA, 160 bp, HOMO SAPIENS
gatctgccttttattcatcagtcttctgaattaggtgacttagatacctggcaggaaaataccaatgcatgggaagaagaagaagatgcagcctggcgagcagaagaagttctgagacagcagaaactagcagacagagaaaagagagcagccgaacaac

SEQ ID NO: 663 - DNA, 50 bp, HOMO SAPIENS
gatcataccattacactccagcccaggcaacaagagtgaaaccctgtctc

SEQ ID NO: 664 - DNA, 124 bp, HOMO SAPIENS
aatgacaagaattgcacagtttattattttgagacaattgttgcagacataaatatttaaaattttctaagcaaggtgcttttaacaaaatttttaaagttggaaagagctgataacttggatc

SEQ ID NO: 665 - DNA, 70 bp, HOMO SAPIENS
gatccacgatacagtgctttggcaaagttaagtgaaaaaagcaagcctttaatgcctataaagtccagac

SEQ ID NO: 666 - DNA, 69 bp, HOMO SAPIENS
gatcctacaccctgagcctcagagcactgctactttttaaaatacttctttctcttaaaagtctttacc

SEQ ID NO: 667 - DNA, 104 bp, HOMO SAPIENS
cggcttatttctgattatatttggagaggccaattttacatgatagcttcaaaccaaccattgtacattaaccaaattttacacaagccatttgaaaaaagatc

SEQ ID NO: 668 - DNA, 158 bp, HOMO SAPIENS
ggtaaaataaatgtatttttaaaatatttttaggaagctgcagaatggcgactgagtgcttcttgataagtctgcagtgttagttctatataggctcccttctgtgcttctgtttttgcaaagaccttgattaaatttattccctcagcagcctgatc

SEQ ID NO: 669 - DNA, 14 bp, HOMO SAPIENS
gatcagcgctttac

SEQ ID NO: 670 - DNA, 163 bp, HOMO SAPIENS
gacaactggtcatcaatttattaaaatagctgacttaagcatctgcaatggtgacttccacctcaactcctggctcaatactgatggaagtaatctgcttaacaatctcagaaggactgtgcaagtcaatgagtcgcttgtgaattctcatctggaaacgatc

SEQ ID NO: 671 - DNA, 69 bp, HOMO SAPIENS
ggtgtgttaaacagctttaatctcgctcattgcggcttctcggcacagtaagaaatattgcacatgatc

SEQ ID NO: 672 - DNA, 101 bp, HOMO SAPIENS
  misc_feature (5)..(5): n = a, g, c or t
atgtnaggttggtttcttttttgagacagggtgtcactttgtcacctaggctggtaccatcttggctcactgcaacctcaacctcctgggttcaagcgatc

SEQ ID NO: 673 - DNA, 89 bp, HOMO SAPIENS
gtctaaaagtgcccaggtgggcttaaggctgccagactgcacgcacatctacagcaacaagggcttctattccatctacaacttggatc

SEQ ID NO: 674 - DNA, 309 bp, HOMO SAPIENS
ggatagatacttaggaatgatattgctgggctacgtggtaaatttatgtttaactttttaacaaactgccaaattgtttcccaaagtggctgcactattttacattcccactagcagtgtatgaggtttcccatttctccatatcctccccaacatttgtcaccgtctgtcttttatacagtacccattctaacaggtatgaatggtaatttgttgtggttttaagttttatgttgaggaggaagaatgaggaaaaaattcctaaaacatcaacgcagagaaactggcactaatgtaaacacatgatta

SEQ ID NO: 675 - DNA, 78 bp, HOMO SAPIENS
gaaatcttccttggcttgcattattttatttttcttttccctgcgttcttcctctgccctggtctttacatactgatc

SEQ ID NO: 676 - DNA, 97 bp, HOMO SAPIENS
ccttccatcatttatttaggaaaaagttttatgtattagggtaaagtggtagaagttaacctagaatctaataatctccaatcacccatccctgatc

SEQ ID NO: 677 - DNA, 30 bp, HOMO SAPIENS
tagcctgggccacagagcaggctccacctt

SEQ ID NO: 678 - DNA, 134 bp, HOMO SAPIENS
gatccaccctcctcggcctcccaaaatgttgagattacaggcgtgagccaccacacccggcctcccagagccatcttgtcacatgattatttgtatgtccattactatcaatatctctttcttttgttaaaaat

SEQ ID NO: 679 - DNA, 129 bp, HOMO SAPIENS
gctttgatagttatgtcttttaatatgtattagaagaatacataattagcacacatcaaacctgtgatttcacagatatcactacttgggatgaaaatgatataggataacaatgttagacctcagatc

SEQ ID NO: 680 - DNA, 170 bp, HOMO SAPIENS
aaccaatccgttttttaattcttaaattcttgacattacagaactaaactgaaatttattaacattccactcttacatttcttatcgacaaacagaaataaaattcatgagccaaaaaacccaaaacaaaactaaaaacagggaaaagcttataaaactaaatatggatc

SEQ ID NO: 681 - DNA, 160 bp, HOMO SAPIENS
gatctcaggaattacattttccaacagaccaaaaaatgttttcatgtagcagcaatgcagatttggtgaatatttaatatatattttagtatgtatttcactttatgactgacaattaaaaaatattgtttggccaaatagtaaacacccttttgaaacc

SEQ ID NO: 682 - DNA, 34 bp, HOMO SAPIENS
gatcagaaacttgctattccacccacaccaagtt

SEQ ID NO: 683 - DNA, 193 bp, HOMO SAPIENS
gatccacccagttctttaggcaaccactgatagcattttcttaagtattcttccagatatcgtctatgcatatgtaaaagtatctgtctttctccttttaaaaacacaattggtaatatatcatacttgctggtttgcaccttgctttttttgcttaatatatctagtttataatgacctagatatattaagc

SEQ ID NO: 684 - DNA, 48 bp, HOMO SAPIENS
gatcgtgccactgcactcccgcctgggcaacagagcaagactttattt

SEQ ID NO: 685 - DNA, 64 bp, HOMO SAPIENS
tttcttttttttacatatgtgcacagctttagcaaattaaagcccgagataaacgctcgagatc

SEQ ID NO: 686 - DNA, 209 bp, HOMO SAPIENS
agtttgctaaatgccctgtttattctgcaaacaaggcttagaaatgtatagacacaatttgtaggctgagaaatctctgtgtagttgtgacttccatacttccgcaaaagtagtaaggacagcgacttctgaacacattaacctaccactgttttgtttagagcgaacacagtgagaatagattcctaacggcctgtttctctaagtga

SEQ ID NO: 687 - DNA, 70 bp, HOMO SAPIENS
gtttgtggtgattgtttattcaagaatatttggaacaaacatccctgcttaatagttactagttaagatc

SEQ ID NO: 688 - DNA, 44 bp, HOMO SAPIENS
ggaaaatatatttttccatctatgcccacaagtcacgatggatc

SEQ ID NO: 689 - DNA, 98 bp, HOMO SAPIENS
gatcaaaaaattcttggttgtcactcttctgtaaccacaatcttccttctgaactgccacccagtttttacataataataacaagcaatagaaaaacc

SEQ ID NO: 690 - DNA, 150 bp, HOMO SAPIENS
gaaaaagttgcaagccttttaattcattgacctcttttgactgattggaatttttaccattctgtacactctgaattataactaaacaactacatgttactgacaaatttgtgattctttgagtctaaacaacacagaaactagaagatc

SEQ ID NO: 691 - DNA, 193 bp, HOMO SAPIENS
gatcctggctcgccgccagacaattgaggagagaaaagagcgccttgagagtctgaatattcagcgtgagaaagaagaattggaacagagggaagctgaactccagaaagtgcggaaggctgaggaagagaggctgcgccaggaagcaaaggagagagagaaggagcgtatcttacaggaacatgaacaaatc

SEQ ID NO: 692 - DNA, 47 bp, HOMO SAPIENS
agacagttttgctcttgtcgcccaggctggagtgcaatggtgcgatc

SEQ ID NO: 693 - DNA, 144 bp, HOMO SAPIENS
gatcattcatttcaccttggtttctaactttaggtttactttcacttgttatttgacttagcaggtgcaacaaaaacaagaaacaaatgtgcccaccccactttccgcttaactgaaaagcttaaaataaatttctgaattatg

SEQ ID NO: 694 - DNA, 104 bp, HOMO SAPIENS
ggtcaagacaccttcagaaaaatgttgggatctacacctacgtcagtcatttcattgtgttcacaatttgctaattcattttttatcttctctagtatcagatc

SEQ ID NO: 695 - DNA, 69 bp, HOMO SAPIENS
gatctcaacaagagctgtattttaagtatttagacagttctttgttagctggtttctagttggttatct

SEQ ID NO: 696 - DNA, 44 bp, HOMO SAPIENS
gatctctttttacaggttaattttagcatttggtttaaaccacc

SEQ ID NO: 697 - DNA, 97 bp, HOMO SAPIENS
gatcaagctatcattcttgatggtataaaaatggacactggagtagaagtctctgatattggaagccaagatgctcccataatactctcagatagtg

SEQ ID NO: 698 - DNA, 101 bp, HOMO SAPIENS
gatcatatgttaattagtgtaatcattccaccttatattcaaaaatcataaaaccgtattgtaccctataaaaatatacaataatttgtcaatatataatc

SEQ ID NO: 699 - DNA, 74 bp, HOMO SAPIENS
catttattcatattgttttgattaccctcgtgttactacaagatggcaataaatactatgggattgtttgtatt

SEQ ID NO: 700 - DNA, 17 bp, HOMO SAPIENS
taaactgaaatatttct

SEQ ID NO: 701 - DNA, 87 bp, HOMO SAPIENS
ggctatactttcattccaaaagaagagctgaattgatgcttgagttacagcaccagtctattattagtgaatgaagtatattcttgg

SEQ ID NO: 702 - DNA, 142 bp, HOMO SAPIENS
gatcaggaattctaaataaatttcccagttaaagattattgtgacttcactgtatataaacatatttttatactttattgaaaggggacacctgtacattcttccatcatcactgtaaagacaaataaatgattatattcac

SEQ ID NO: 703 - DNA, 30 bp, HOMO SAPIENS
gatctgatagggatttgacttagtggaagg

SEQ ID NO: 704 - DNA, 47 bp, HOMO SAPIENS
gatcaatttgtaattgcttacatttttacaataaataatctgtacgt

SEQ ID NO: 705 - DNA, 141 bp, HOMO SAPIENS
cttctgccgttcttcctttctttctcttttattcttcttcacctccccttgctgttccacggcgtctttcactttggaggctggaaccttggtggagatttctgcatgtggatttgccactgggtggagtggccgttgatc

SEQ ID NO: 706 - DNA, 34 bp, HOMO SAPIENS
gatctccaagaaaaaaagcatattatgtgcttac

SEQ ID NO: 707 - DNA, 33 bp, HOMO SAPIENS
atggtttatccctttattgtttctcctttgatc

SEQ ID NO: 708 - DNA, 141 bp, HOMO SAPIENS
acacagaatacaaataaaagtttattgtacaaaattaaggccttcgtatttcatagttcaccaaggctaaagaaacactggataatttaagtccttcacaaaaatttcacttttactcctcctcatctctaactattaaaa

SEQ ID NO: 709 - DNA, 105 bp, HOMO SAPIENS
gatctgtccacttctggtgtcaaagattttactcatcttcttagtacattctatgtattttatatgtataattttatacaattaaaaatagatttttgtctagtg

SEQ ID NO: 710 - DNA, 118 bp, HOMO SAPIENS
cccaggtcctgtgcagtcccaggtcctgtgatgtttattgaaggaagcaagggcagggctggggtgccgccctcaggcctggatgtggctcttcgcactgaaggccaagtagtagatc

SEQ ID NO: 711 - DNA, 93 bp, HOMO SAPIENS
ctttgggagacagagtctcactctgtcacccaggctggagggtagtggtgcattcatagctcactgcagccttcaactcctgggctcaagcga

SEQ ID NO: 712 - DNA, 27 bp, HOMO SAPIENS
gatctgacccctattgtggtagaagac

SEQ ID NO: 713 - DNA, 44 bp, HOMO SAPIENS
aaatggagtctctgtcacccaggctggagtgcagtgacacgatc

SEQ ID NO: 714 - DNA, 77 bp, HOMO SAPIENS
gatcgtaatgtaaaattcttttaccatgtcaagaaattattaaaaatacaggtactttgacctctttctaaagccgc

SEQ ID NO: 715 - DNA, 74 bp, HOMO SAPIENS
ttttgtttttttgttttttttcttgagacagaatcttgctcttgtcacccaggctggagtgcaatggcgcgatc

SEQ ID NO: 716 - DNA, 66 bp, HOMO SAPIENS
gttggtactcccttagactgaatgttagggaccaaaaaaataataaaataattaaaatgaacaagg

SEQ ID NO: 717 - DNA, 83 bp, HOMO SAPIENS
gtttaaaatgaaaagtttatttgctgagtacaccaaataggatgcaagcactgtcatgacaaatatacagaaatatgcagatc

SEQ ID NO: 718 - DNA, 101 bp, HOMO SAPIENS
gatctgtgtgctcttatcaccaatcagttcagacctggttgattttgtactttggaactgtaccttggatggttttgtttattaaaagagaaacctgaagt

SEQ ID NO: 719 - DNA, 60 bp, HOMO SAPIENS
gatcacttgaggtgaggagtcgataccagcctgggcaacatagcacccgtctctacagag

SEQ ID NO: 720 - DNA, 134 bp, HOMO SAPIENS
gatccatcgcagagtcctaaagaagaacccactgaaaaacttgagaatcatgttgaagctgagcacgtgaagctcgctggttctgtgcgaagggtattcctggtgctgaataaagggtgttgctgtcaaggctg

SEQ ID NO: 721 - DNA, 71 bp, HOMO SAPIENS
caactgaatcaaaacagtgttaaattatggcaatatttgcactttgggaatgagtacataactgtatgatc

SEQ ID NO: 722 - DNA, 33 bp, HOMO SAPIENS
gcttattcagtcgctttattaataaaagagatc

SEQ ID NO: 723 - DNA, 140 bp, HOMO SAPIENS
cgtccttttccccggttgctgcttgctgtcctggtccgcgccagagcccagcgcgcctcgtcgccatgcctcggaaaattgaggaaatcaaggacttcctgctcacagcccgacgaaaggatgccaaatctgtcaagatc

SEQ ID NO: 724 - DNA, 143 bp, HOMO SAPIENS
gttattaatttctacctcaattccatgataatcagataacatactctgaatgatgtcaatcatttgatatttactgatgcttttttatggctcagtatatggttatttatgatacatgttttatggcgttgttgggtgtgatc

SEQ ID NO: 725 - DNA, 63 bp, HOMO SAPIENS
gatcacttgagtccaggagttcaagaccagcctgggcaatacagtgggacccccatctctacc

SEQ ID NO: 726 - DNA, 198 bp, HOMO SAPIENS
gagaatgaaatgtttcccatcatagtggattcttaagcacgttctccacgtatgcggcgtgctagctggatgtcttttggcataattgttacacgtttggcatggatagcacacaggttggtgtcttcaaaaaggccaaccagataggcctcacttgcctcctgcaaagcaccgatagctgcgctctggaagcgcaga

SEQ ID NO: 727 - DNA, 49 bp, HOMO SAPIENS
gatccattttaaaatttgtaattcaataaagttttttttgttgttaaac

SEQ ID NO: 728 - DNA, 37 bp, HOMO SAPIENS
gatcaataaagaggccaaatttttagctgttttatgt

SEQ ID NO: 729 - DNA, 65 bp, HOMO SAPIENS
ttttttaactttttaagagatgaggtctgacgctgtcactcaggctggagtgcagtggcacgatc

SEQ ID NO: 730 - DNA, 60 bp, HOMO SAPIENS
gctataaaggccattatgtgaacaactggcaaaatctgaaagaggtctgctgattagatc

SEQ ID NO: 731 - DNA, 85 bp, HOMO SAPIENS
ctacattctcctttggttcagtgcctgactttgtagaatgctgatttgtagtttcttgtggtcgctcctcttcagtctttttcat

SEQ ID NO: 732 - DNA, 26 bp, HOMO SAPIENS
gatcaagtcctcagtggtacataaac

SEQ ID NO: 733 - DNA, 46 bp, HOMO SAPIENS
gatcgaagatgtgggttcagatgaggaggatgacagcggtaaggat

SEQ ID NO: 734 - DNA, 102 bp, HOMO SAPIENS
gatccagcaaacaggttctgtttaagaaaaataatttatactaaatttagtaaaatggacttcttattcaaagcatcaataattaaaagaattattttaatg

SEQ ID NO: 735 - DNA, 177 bp, HOMO SAPIENS
gatcatgttatattctatatcagacaaactattttcttttgacctttcttcccctccatccagtatttcggttgatttcattttctcccctctcttccccttccacgaactgcaataccagtaaccttggtatatattttttgatactgtacacatggatgtcttgtttctatgtgc

SEQ ID NO: 736 - DNA, 160 bp, HOMO SAPIENS
gatctgggcgattctgagccatgccatttttaccttatgtctgctagaaagtgttgtagttgattgaccaaaccagttcataaggggaattttttttaaaaaacaacaaaaaaaaacatacaaagatgggtttctgaataaaatttgtagtgataacagt

SEQ ID NO: 737 - DNA, 77 bp, HOMO SAPIENS
gatcattattttgtgtaaaatgctataaatatttttttatgtttgttttctctaaataaactgtgagtttctaaagt

SEQ ID NO: 738 - DNA, 104 bp, HOMO SAPIENS
cttttgtcagcacacaagatgagcttcctgcatggagttatgaccgtaaaactgttgttgacgtttttggtactggattcagctacgctggcatctgtcagatc

SEQ ID NO: 739 - DNA, 74 bp, HOMO SAPIENS
gcgggaaaatattttattgctgccatccccatggtgagccgctgggggtgaggggtgaagctgggtggtggatc

SEQ ID NO: 740 - DNA, 210 bp, HOMO SAPIENS
gaaaaaccatacatcctttttattgttaagtcataaagaggtatcaaaattaaaagcaaaaattacagggtaagacttaacaaaactactaggagcgtcaaaggaagtgaaaatgggactaggcgcggggcaatatgaattaatgaacatgggaaggacaaggatggggagaacagtgagcatgtgctaaagatactaggggagaggatc

SEQ ID NO: 741 - DNA, 57 bp, HOMO SAPIENS
ggtaattaccaagacatttattagttgtcaaaaagctttacaatcagtttcatgatc

SEQ ID NO: 742 - DNA, 134 bp, HOMO SAPIENS
gatcattatgtgcaaaacaccaagttttaaaaataactcacagaatggccttagttttcaatgtctgatggtatatttgtgagttgtgttatctgtaatatacatcccagaataaaggagtgaattaaatagtt

SEQ ID NO: 743 - DNA, 55 bp, HOMO SAPIENS
gatcataccactgcactccagcctgcacaaagcaagactctgcctaaaagaaaac

SEQ ID NO: 744 - DNA, 140 bp, HOMO SAPIENS
gatcccactgcagatgtttttgtaacaccagctgaggagaaaccaatagaaatacagtgggtaaaaccagagccaaaagttgatttgaaagcaagaaagaaacggatttacaaaaggtaaagaaaaatgaaacagaggat

SEQ ID NO: 745 - DNA, 131 bp, HOMO SAPIENS
gatctcactaaaggatttctatttgctgtcagttaaaaataaagccctaaatacatttttattctttctactgagggcattgtctgttttctttgtaaatgccgtacaataaacaaattatttaataaccc

SEQ ID NO: 746 - DNA, 72 bp, HOMO SAPIENS
ttttttaaaaagacacaacatgctcaagggtacacagaatatggaaaaagtcatcacttcatttgagagatc

SEQ ID NO: 747 - DNA, 114 bp, HOMO SAPIENS
caaaaaagaaatcttttaataaaaattactcataaaaatcctaataaattttaaagagcaagatattccttattacatttataaaagaacatttggtccttttacaaaaagatc

SEQ ID NO: 748 - DNA, 112 bp, HOMO SAPIENS
agtttagctagaggtattttttgttaattttttcaaaaaatccagctcctggattcactgattttttgaagggtttttttgtgtttctatctccttcagttctgctctgatc

SEQ ID NO: 749 - DNA, 122 bp, HOMO SAPIENS
  misc_feature (117)..(117): n = a, g, c or t
gatccagtgacagcaggtgtcatgggtcaagcataaatcatatatagcattttcaggcatgttcctggtagttcttttgagtctgacattctaataaaataatttgtagaaaaaaanaaaaa

SEQ ID NO: 750 - DNA, 127 bp, HOMO SAPIENS
gacaatttctgagattaaactttattaaagttacttttccagaaaattcagtaaatatggtaatataggaacaaacttaggctcataagccttttaactttttacataatctttaatgatgttgatc

SEQ ID NO: 751 - DNA, 42 bp, HOMO SAPIENS
gatctttggaaggagaagatgctgcaggacaccaaggatatc

SEQ ID NO: 752 - DNA, 56 bp, HOMO SAPIENS
acgaagccagtatcttgaaactattggcaattgcaagtgtctttaaactagggatc

SEQ ID NO: 753 - DNA, 39 bp, HOMO SAPIENS
gatcttcccattcacagagttagtaaacctctgtactac

SEQ ID NO: 754 - DNA, 24 bp, HOMO SAPIENS
gatcacaccgtacttgaggccatc

SEQ ID NO: 755 - DNA, 49 bp, HOMO SAPIENS
gatcacgccactacactccagcctaggtgacagagtgcgactctgtctc

SEQ ID NO: 756 - DNA, 105 bp, HOMO SAPIENS
gatcttcagctctgcgaaattccttcgctttttcttaagggtttctggcacagcaggaacctccttcttcttctcttctacaccctccatggttccagccggaaa

SEQ ID NO: 757 - DNA, 194 bp, HOMO SAPIENS
aagtctggcaaggatttattaggaagcttattagtcacagtgaataaaagccatgaaaagaagaactcaaatctccaaattctggcatcggacttacaacactagttagaagctaataaacattaaggaatttccaaggggaatcttaactaagtctcaatcttactgaccagattacccacacacatagaaat

SEQ ID NO: 758 - DNA, 51 bp, HOMO SAPIENS
ggattttagtagagatggggtttcacaatgttggccaggatggtctcgatc

SEQ ID NO: 759 - DNA, 14 bp, HOMO SAPIENS
gatcacatgaaaat

SEQ ID NO: 760 - DNA, 50 bp, HOMO SAPIENS
gatccccggtggttttgtgctcaaaataaaaagcctcagtgacccatgag

SEQ ID NO: 761 - DNA, 59 bp, HOMO SAPIENS
gatcttattaaactgctggtctgactttatggattgacactgttcctttcttttattgc

SEQ ID NO: 762 - DNA, 40 bp, HOMO SAPIENS
tttggaaatttcagcgtttttgcattggtttttcctgatc

SEQ ID NO: 763 - DNA, 134 bp, HOMO SAPIENS
gatcaggaggcccacttggatttatagtatagcccttcctccactcccaccagacttgctcatttttcgagtttttaactagactacactctattgagtttaattttgtcctctaggatttatttctgttgtcc

SEQ ID NO: 764 - DNA, 48 bp, HOMO SAPIENS
gatcacgccactgcattccagcctggtgacagagcgagactctgtctc

SEQ ID NO: 765 - DNA, 127 bp, HOMO SAPIENS
gatctggaccgtcccctgcccacttgctccccgtgagcactgcgtacaaacatccaaaagttcaacaacaccagaactgtgtgtctcatggtatgtaactcttaaagcaaataaatgaactgacttc

SEQ ID NO: 766 - DNA, 106 bp, HOMO SAPIENS
gggttgaaagtcgttttatttcattggtaacaacaacacagtgggaaattatgtattacagagtagagacacatattcctgtttaaaatgaggaaacaagaagatc

SEQ ID NO: 767 - DNA, 109 bp, HOMO SAPIENS
ccaaaaagaaagaggctttttattacattgttacaggcaatgcattacgtgagctggtcccgtgtacaatccccaagccacggcaaaacatcaggagaacgcaccgatc

SEQ ID NO: 768 - DNA, 159 bp, HOMO SAPIENS
gatcgcttgtagctgggaacagtgcttacttcagggaaaactgattttgatagaaccgactagttgagtattaaactgttttggaaataatttatatatctaagtgctgaaatatatttctcttctccccacttattcaaataaaaaagttttaatact

SEQ ID NO: 769 - DNA, 48 bp, HOMO SAPIENS
gatcacaccgctgcactccagcctggtgagtgagcgagactccatttc

SEQ ID NO: 770 - DNA, 51 bp, HOMO SAPIENS
cagttttttacaactcaggtgagttttagctttattggggagggggtgatc

SEQ ID NO: 771 - DNA, 198 bp, HOMO SAPIENS
agaaggagaacaattttattctaaaaatagaacttggtaacaatgaaataccaaaagctggtcattataataaaaagaaaagaagagtttaacttttttttgtgaaaatacaaaattatcactataatatactgccaactctgtttatctgtatttgaattattcctttcatggattattcaaggaaattttaaaatt

SEQ ID NO: 772 - DNA, 44 bp, HOMO SAPIENS
gatcagaagagctttccttgttaaagccgtctggtgactatttt

SEQ ID NO: 773 - DNA, 68 bp, HOMO SAPIENS
gatcacgaggtcaggaaatcgagaccatcctagctaacatggtgaaaccccgtctctactaaaaatac

SEQ ID NO: 774 - DNA, 87 bp, HOMO SAPIENS
ctctgcctgactcttccttcctgtttcttgcaacccagggatgaaggactgccctcctaacttattataccctccttgcccaggatc

SEQ ID NO: 775 - DNA, 83 bp, HOMO SAPIENS
gatctagacatcaacctagatgcatcatttaatttaatttaaggtaaaccagcaattccattttttgttgccgacaatgaatt

SEQ ID NO: 776 - DNA, 71 bp, HOMO SAPIENS
gccggtctcagcctttaatcgtggcagggcctcacgcacgcgcgcacgtacacacactcgggcttcagatc

SEQ ID NO: 777 - DNA, 39 bp, HOMO SAPIENS
gtttacttctgcaaagggtagcttcttaaaaacgtgatc

SEQ ID NO: 778 - DNA, 24 bp, HOMO SAPIENS
gatcttcttacagccaaccttgct

SEQ ID NO: 779 - DNA, 108 bp, HOMO SAPIENS
gatcttaggctctgctctcagcagatttcaggtgtaaccatttgttaactgtactgaaggtgtgtcctcaagaagaaagtgttcaaattaaaaaagctgctgccaagt

SEQ ID NO: 780 - DNA, 108 bp, HOMO SAPIENS
gatcccttgagcccgggatgttcgcttcagcctgggatgttgaggctgcagtgagccgtcattgtgccactggactccagcctgggtgccagagtgagaccctgtctc

SEQ ID NO: 781 - DNA, 60 bp, HOMO SAPIENS
ggagacagagtattgctatgttagaccaggctggtctacagctcctggcatcaagggatc

SEQ ID NO: 782 - DNA, 73 bp, HOMO SAPIENS
gatcaagtggaagaaaggatatcagtgactgaaaatcaaattaatgacataaagcaagatgacaagattggag

SEQ ID NO: 783 - DNA, 27 bp, HOMO SAPIENS
gatcccccgattacatttctaagcgtg

SEQ ID NO: 784 - DNA, 33 bp, HOMO SAPIENS
gatccagcatgaaagccaacatgaccgcagagg

SEQ ID NO: 785 - DNA, 38 bp, HOMO SAPIENS
gatccccgtgttactgcctatcagcattttactacttt

SEQ ID NO: 786 - DNA, 108 bp, HOMO SAPIENS
gatcagttgagatggaaaataatgtcctgcaagatgaggattgaacagttaaaacaaacaatatgtaaaggaaatgaagaaatggagaaaaattctgaaggccttctc

SEQ ID NO: 787 - DNA, 128 bp, HOMO SAPIENS
gatcggcgcactgcgagcagtagcccaaacaatctcatatgaagtcaccctagccatcattctactatcaacattactaataagtggctcctttaacctctccacccttatcacaacacaagaacacc

SEQ ID NO: 788 - DNA, 201 bp, HOMO SAPIENS
caaatatacttttccaattccacagtaggccaagcaatcaaggccctcactggggtagtgttcaataatcctcttaaactggttttcggcttcaaataattcctcaggctgccctattccaaggagggaaatggcaagtccatatacgaccaaaacatagttaatcatggccaggttcaattgctttattttttgaggatc

SEQ ID NO: 789 - DNA, 59 bp, HOMO SAPIENS
gatcccatagaagtaccagggtgctttttttttttcctttcaggggtcattgtttgcat

SEQ ID NO: 790 - DNA, 48 bp, HOMO SAPIENS
gatcatgccattgcactccagcctgggcaacaagagtgaaactccatc

SEQ ID NO: 791 - DNA, 157 bp, HOMO SAPIENS
gatcttgcctctttcctcctcatgaaagcagcacacattgtgttaacttatgtctcttgttaaatgagcttaatgtctttgtgttttgtccaaaactgtattgaaaaaatattgtttaatgcaaatgaaggaatgcaataaagagtaaatatacttg

SEQ ID NO: 792 - DNA, 49 bp, HOMO SAPIENS
gatcacgccattgcactacagcctgggcaacaagagcgaaactttgtct

SEQ ID NO: 793 - DNA, 124 bp, HOMO SAPIENS
agggaagaaacaatcttatttattaatggcctgcatatttagaaagggtttaaaaacacatacatttcctgggacacaaacttacttaccaaatgtatgtccttacaaaataacttcacagatc

SEQ ID NO: 794 - DNA, 110 bp, HOMO SAPIENS
gatctccagagacccaagtttaggttctcatagtgtatttgaagtagttatactcctggcttaagtagtttagtgcctgggagaatccattactgaaaagcatttaactt

SEQ ID NO: 795 - DNA, 118 bp, HOMO SAPIENS
aaatgaattattctttaggaactgctcaatatcacccaagcatagttacaaagtatctcttcaaattatacatactcttgaggtcaaaaacgtcaaactatttccgtaatgtaagatc

SEQ ID NO: 796 - DNA, 152 bp, HOMO SAPIENS
gatcatggaggaaaaaggtactggataaaagtaaacttcaaaccttagggcgggagcactaaaaccaaaatacatgtattatttatagaaaatattttctgttttaatcttttctttttaaacaaggactcatacttaaaaaaatgtttagc

SEQ ID NO: 797 - DNA, 275 bp, HOMO SAPIENS
gatccattgatttgtagacctgtgcctcgcataatacttaaactaactagactacaacataaaaatatacacttaataatagattataaaaataacataatatggtattggttaactgtaaaaaataatagaacttttggaataaatatagttagttggttaatgttagatagaatataaaaagattttgtattttaaaataaatatagttagttggttaatgttagatagaatataagaagattttgtatttggggaataaagagggtggttgg

SEQ ID NO: 798 - DNA, 33 bp, HOMO SAPIENS
gaaaacaaagtgagcctttattaatattagatc

SEQ ID NO: 799 - DNA, 148 bp, HOMO SAPIENS
gcagaccataggaagtttattgtgtctttgagacactacaatgcagactcctcttccagagaaaggtctttcagagacagggaaccgtcatccggatgcgttgctcatttgtcattttcataggcagctggattcttcctcttggatc

SEQ ID NO: 800 - DNA, 74 bp, HOMO SAPIENS
cgatcttaagtcatacattttaattgtgtacaggttgtacaactgaaggaataaatgtctattaaactaaagaa

SEQ ID NO: 801 - DNA, 48 bp, HOMO SAPIENS
gatcgcattgctgtacccagcctgggccacagtgcaagactccatctc

SEQ ID NO: 802 - DNA, 124 bp, HOMO SAPIENS
aaaatgtacttcctccagacagaacagctggctgatttgaatataatagctatatgtatttaatgcatttcctcaactgtaaaatgtaccattgatttaataaaaagtccttggtaagaagatc

SEQ ID NO: 803 - DNA, 56 bp, HOMO SAPIENS
ctattacttccttctttttacaaacccttgtgtcgagggctgactttcaatagatc

SEQ ID NO: 804 - DNA, 106 bp, HOMO SAPIENS
aggttcacatgtatataattacaaatggtgataagttctatgaatcaggagaagatgcagttctatagcagagacctagtttcgttcagcgtgggaggaggtggtg

SEQ ID NO: 805 - DNA, 41 bp, HOMO SAPIENS
gatctaatctataattgtgacaaaactataccgagagaatg

SEQ ID NO: 806 - DNA, 134 bp, HOMO SAPIENS
gatctaataaatgtcaaccaacattgacattgtaattgggtgattacaataaaaggtgagcagtttgttgtttattaataattagcttttgcaggtaatgaaatagcagggaagtaacatgctgctttaggact

SEQ ID NO: 807 - DNA, 64 bp, HOMO SAPIENS
gatcagccctcaaagaatggggtctgtgggctggttaaaatagaaataaacttttaaaatatcc

SEQ ID NO: 808 - DNA, 16 bp, HOMO SAPIENS
gctctccgtgaaaaac

SEQ ID NO: 809 - DNA, 117 bp, HOMO SAPIENS
ggcgtaacgagactttactgaaaacagaaaaccgggcgaaccaaggattacaaacaggaatgtggactcgtagcaaaacaaaacaaaacagaacaaaaaagggaggggggaccgatc

SEQ ID NO: 810 - DNA, 83 bp, HOMO SAPIENS
aattttttagtagagacaggggtctcactatgttgcctaggctgtggcttgctttataactattggtgtaccagaataggatc

SEQ ID NO: 811 - DNA, 211 bp, HOMO SAPIENS
gatcttaagtttcatgaggaataggtttgcttcattgaaactggtttttatcttcccaaatagttttcaatcattcctttaaagtactaggttgattaatattgcaaagttgtaatgtaaaatgtgaccttacctctgtctaacctgtattgttcacatgaatgggagtttgactttgggaataaacattgaaaatttgcagggaggaccc

SEQ ID NO: 812 - DNA, 146 bp, HOMO SAPIENS
gatctttggtttttgctttaatttattaaatgtattttccatactgagtttaaaatttattaatttgtaccttaagcatttcccagctgtgtaaaaacaataaaactcaaataggatgataaagaataaaggacactttgggtacc

SEQ ID NO: 813 - DNA, 80 bp, HOMO SAPIENS
cctataagataatcgtattgttgctgtaaaagccgtaattcttttattttttttaattcccttaaagaaatcgtgtgatc

SEQ ID NO: 814 - DNA, 98 bp, HOMO SAPIENS
aagagtctctagagtttttattattcaaaatgagtttcatttcaattcaggtacttgaaagaaggttctagtctgttactttgacacagcacaagatc

SEQ ID NO: 815 - DNA, 54 bp, HOMO SAPIENS
gatcgcgccactacactcccagcctgggcgacagggtgagactgtctcacaaag

SEQ ID NO: 816 - DNA, 139 bp, HOMO SAPIENS
gatcaaccttaaaggaaactgctatcgaacttggctatctcacagcagagcagtttgacgaatgggtaaaacctaaggacatggtgggtccaaagtgatttacataaatttataatgaaaataaacatgtataaaattt

SEQ ID NO: 817 - DNA, 104 bp, HOMO SAPIENS
gggttcaaatcaccgttctgtctactttatttttatttattttattttatttttgagacggagtctcgctctgtcgccccagactggagtgcagtggcgcgatc

SEQ ID NO: 818 - DNA, 121 bp, HOMO SAPIENS
gagacagggtctcactctgtcaccaagtttggagtgcagtggtatgaactcagctcactgcaacctccacctcccaggctcaagcaatcctcccaactcagccttccaagtagctgggatc

SEQ ID NO: 819 - DNA, 24 bp, HOMO SAPIENS
gatccagccatcatgaagaaattg

SEQ ID NO: 820 - DNA, 49 bp, HOMO SAPIENS
gagacagggtcttgttctgctacccaggctggagtgcagtggcatgatc

SEQ ID NO: 821 - DNA, 56 bp, HOMO SAPIENS
aaggtctcatagacaataaaatggcagcaactattttactgactagtaccatgatc

SEQ ID NO: 822 - DNA, 56 bp, HOMO SAPIENS
gatcaagaatcctgctccactaagaacggtgctaaagtaaaactagtttaataagc

SEQ ID NO: 823 - DNA, 114 bp, HOMO SAPIENS
gatctctgagtaaagaggccgtttatctttgtaaacacgaaacatttttgctttctccagttttctgttaatggcgaaagaatggaagcgaataaagttttactgatttttgag

SEQ ID NO: 824 - DNA, 93 bp, HOMO SAPIENS
gatctccaaagtggtgagtttatgtgtgatttttattttgtttatgctcttctgtattttccgaatttcatacaataaatatctgttactttt

SEQ ID NO: 825 - DNA, 136 bp, HOMO SAPIENS
gatccacctcaaagggatgaatttttgggaacttgcgtgcaacatcttaattcttacttaggttttcaaatatacagaaaaattgaacaatacaacaaacatctgtataataataaactgtataataatgaactgt

SEQ ID NO: 826 - DNA, 62 bp, HOMO SAPIENS
agtagacacggggtttcaccatgtcggccaggctggtctcgaactcctgacctcaggtgatc

SEQ ID NO: 827 - DNA, 77 bp, HOMO SAPIENS
acgttttgtatgtttttttatttgctccaggtggggttttgactgtcactttcccacactctggattagttctgatc

SEQ ID NO: 828 - DNA, 37 bp, HOMO SAPIENS
gatcacaccactgcactccagcctgggctcaaaaaag

SEQ ID NO: 829 - DNA, 55 bp, HOMO SAPIENS
gatcgcaccattacactccagcctgggcgacaaagcgacactccatctcaaattc

SEQ ID NO: 830 - DNA, 72 bp, HOMO SAPIENS
gatcaaaacattactgcaaacatgtatgatgtttataggctttacaataaaactactgggtacaataaaaac

SEQ ID NO: 831 - DNA, 44 bp, HOMO SAPIENS
ggacttagtagagatggggtctcactatgttgcccagactgatc

SEQ ID NO: 832 - DNA, 150 bp, HOMO SAPIENS
gatcctagtaacaccaaactcaaagtagctcctggtcagaaagtgcagtaaagggtgcatgaaagcatccgctctatggtctcctcagtggtctattttaggtgtggtttttttttttttttttactgctaaactatattaactcataat

SEQ ID NO: 833 - DNA, 100 bp, HOMO SAPIENS
gatcttaaggtatcagtatttcagaatcctgcgacgattttatttctaaattcatgtactgtatgtccataagtgaaaataaaatgtcatattcttttct

SEQ ID NO: 834 - DNA, 204 bp, HOMO SAPIENS
cgcaagttgttttatttacaatgccaacttttaaaaggtcactaaagttaactggacaataaactaggcagacattgctttacaaaaaagagggaaagcccaaaatgccacttatagagaacccacagttcagttttattcagagcaaagaaaaaagaaactttccatcatactagaagaaacttggccgtaaggtttgggatc

SEQ ID NO: 835 - DNA, 110 bp, HOMO SAPIENS; identical to SEQ ID NO: 37

SEQ ID NO: 836 - DNA, 75 bp, HOMO SAPIENS
gctttccaaaatactgcagtttaattctgaaaaactcaaatgcaagagacccagagctgtctatgggattggatc

SEQ ID NO: 837 - DNA, 56 bp, HOMO SAPIENS
gatctgttcatggtcacatctgtaccctcaacatatatcccttgtgcgacaaaaaa

SEQ ID NO: 838 - DNA, 68 bp, HOMO SAPIENS
gatcacgaggtcaggagattgagaccatcctggctaacatggtgaaaccccacctctactaaaaatac

SEQ ID NO: 839 - DNA, 146 bp, HOMO SAPIENS
attccaaaaataattttatttaataggtattaaataatgtatagaaggaaaaggagctggtgtcaggttctgtttacgtccttctcttaccctagctcttctcgtgttttgcctatttttttgggcattttcttagcatggggatc

SEQ ID NO: 840 - DNA, 65 bp, HOMO SAPIENS
ttttttgtcatgttggagttatgaggggaatggggagttgtttgtttccatttgtaaactcgatc

SEQ ID NO: 841 - DNA, 87 bp, HOMO SAPIENS
ggatgtattgtatacacagtggaaagctggttttatttgggagacaatgggagcttttacattgttgagcaaaggagtgacgagatc

SEQ ID NO: 842 - DNA, 94 bp, HOMO SAPIENS
gatcccagcattttgggaggctgagacgggtggatggcttgagcgcaggagtttgagaccagcttgggcatcatagcaagaccctatctctatt

SEQ ID NO: 843 - DNA, 81 bp, HOMO SAPIENS
gatcaaatttgaacttcattttggggggtattttggtactgtgatggggtcatcaaattattaatctgaaaatagcaaccc

SEQ ID NO: 844 - DNA, 203 bp, HOMO SAPIENS
gatccacagtggagttttagtaattatattttgttgatttcttcattttgttttctggtataaaagtagagataatgtgtagtcacttctgatttagtgaaaccaattgtaataattgtggaaatgttttgtctttaagtgtaaatattttaaaatttgacataccctaatgttaataataaaaagaactatttgcatattgc

SEQ ID NO: 845 - DNA, 89 bp, HOMO SAPIENS
aacttatttatttatttatttatttatttatttatttattgagatggagtctcgctctgtcgcccgggctggagtgcggtggcatgatc

SEQ ID NO: 846 - DNA, 79 bp, HOMO SAPIENS
gatcttagattttgttattgcccaagatttaggatgctttgcattgtgtattcagcttattagctttgtgttaattttg

SEQ ID NO: 847 - DNA, 88 bp, HOMO SAPIENS
aaaccttaacccagagttatttttattttccagaacgtgttaggaactagtacttaaataatctcaagtccctgaggggccagagatc

SEQ ID NO: 848 - DNA, 92 bp, HOMO SAPIENS
acagcttgtactttactttaatgtgtaatactcaactcaaggtacaagacaattgcatttaacattgttataaataaaaggaacatcagatc

SEQ ID NO: 849 - DNA, 35 bp, HOMO SAPIENS; identical to SEQ ID NO: 76

SEQ ID NO: 850 - DNA, 120 bp, HOMO SAPIENS
gatcagaaaaaagtacctcagaagaaagttccttatgcaaaacccattcctgctcagttccagcaggcttggatgcaaaataaagttccaattcctgctccaaatgaggtgctgaatgac

SEQ ID NO: 851 - DNA, 89 bp, HOMO SAPIENS
agtcttttaatgttagccttttaatattttccaataagtgctttcaactcagcaatatacatatcatgctttcctcattattattgatc

SEQ ID NO: 852 - DNA, 34 bp, HOMO SAPIENS
gatcagttttttataaaaatatatatttttgtcc

SEQ ID NO: 853 - DNA, 77 bp, HOMO SAPIENS
gatcatgccactgcactccagcctgggcaacagagcaagactctgcctcaaaaaaaaattaaaataaatttaaatac

SEQ ID NO: 854 - DNA, 89 bp, HOMO SAPIENS
gaaacagggtctcactctgtcacccaggctggagtacagtggtgccatcctggctcactgcaacttctgcctccagggctcaagtgatc

SEQ ID NO: 855 - DNA, 189 bp, HOMO SAPIENS
gaatgtttaattctcaagaaaattgtaatcaattagtaaaaattataaaatgttaatagtattaaagcttgagtcttacattgcatgtttttttgtatccacttgaggaaacattacattctacaaaaagtggcatttccattttctatttattctctttaattgtttttcaaagtttgtatgcagatc

SEQ ID NO: 856 - DNA, 93 bp, HOMO SAPIENS
gatcagccacatcaactcagttgtccaccacaggggaattttgaatgtcttttgtttttgttttgttttgaaaaataataaacaggcaactgt

SEQ ID NO: 857 - DNA, 84 bp, HOMO SAPIENS
gatccaaacagcccatgcttctcatcatgcatcaagatgcgtcaacctcttatttgaaactaaagagaacaaacaagcaacttt

SEQ ID NO: 858 - DNA, 110 bp, HOMO SAPIENS
gatcagtagatttaatgtaatcccaatcaacagcccagcaggattttcataaaaatcaatatgttgattctaaaatatttatggtaatacaaagaaacttgaatatccac

SEQ ID NO: 859 - DNA, 55 bp, HOMO SAPIENS
gatccgttattcaaatggccttataggcattcaataaaggtgaaatgttaaatac

SEQ ID NO: 860 - DNA, 43 bp, HOMO SAPIENS
gatccaccaaaagaaaaaagagccagaaggggccagcaaaaac

SEQ ID NO: 861 - DNA, 137 bp, HOMO SAPIENS
tttgctgtaggcccgggtggttgctgccaaaatgggcaagttcatgaaacctgggaaggtggtgcttgtcctggctggacgctactccggacgcaaagctgtcatcgtgaagaacattgatgatggcacctcagatc

SEQ ID NO: 862 - DNA, 70 bp, HOMO SAPIENS
gatctcaggaggaatacctgagccatggttgtgccactgcactcctaggtgacagagcaagacctgtctc

SEQ ID NO: 863 - DNA, 74 bp, HOMO SAPIENS
gatccttggagatttctcaatcttacactactacacaaagagaatctagtgattacagtgtcccttgcaaaaaa

SEQ ID NO: 864 - DNA, 82 bp, HOMO SAPIENS
gagacagtctctgtcgccaggctggaatgcaatggcttgattttggctcactgcagcttccgtctcctgagctcaaatgatc

SEQ ID NO: 865 - DNA, 107 bp, HOMO SAPIENS
gttgtgttttttttacagtggtgctggacatgcagtcccactcactagacattttggagcattacgtggaaatgcccatccagcggttggagaggtagggtaggatc

SEQ ID NO: 866 - DNA, 45 bp, HOMO SAPIENS
aatgttacaatccactaagtttattacataatccactaatggatc

SEQ ID NO: 867 - DNA, 40 bp, HOMO SAPIENS
gatccttttatcctgctacagtctttagggagctaaaaaa

SEQ ID NO: 868 - DNA, 49 bp, HOMO SAPIENS
gatcatgttactgcactccagcctgggtgacagagtgagattctgtctc

SEQ ID NO: 869 - DNA, 93 bp, HOMO SAPIENS
gatctcactaaaaatatacagctacttcccagctaatctccacccaatgaatgatgatggtatagtatgtgcataatggaaagttaccttact

SEQ ID NO: 870 - DNA, 63 bp, HOMO SAPIENS
tttttccccaaggattattcccaaactatgtacaattaatgggttgccttgcagagttggatc

SEQ ID NO: 871 - DNA, 58 bp, HOMO SAPIENS
ggcactggaagagtttagtgtttcttgtatgcttgaaaataaagtatgtactgttttg

SEQ ID NO: 872 - DNA, 62 bp, HOMO SAPIENS
gatcctagacagcgccttatctatgattgagtgtccgtgtaaataaattcctacttagactt

SEQ ID NO: 873 - DNA, 164 bp, HOMO SAPIENS
cacaagactagatggagaattaatgaagaagaaatagaaatgaggcttatttaaggtacaaaattcttgtatttgtaataacctagatttatagatataaaacaagtcttttaaagacttccctaaaatactcaatataaaacaggagttacttatcctagatc

SEQ ID NO: 874 - DNA, 204 bp, HOMO SAPIENS
aagaccctcatcaatagatggagacatacagaaatagtcaaaccacatctacaaaatgccagtatcaggcggcggcttcgaagccaaagtgatgtttggatgcaaagtgaaatattagttggcggatgaagcagatagtgaggaaagttgagtcaataatgacgtgaagtccgtggaagcctgtggctacaaaaaatgttgatc

SEQ ID NO: 875 - DNA, 55 bp, HOMO SAPIENS
actctggggacgtttattaaggctttggcaacactggggagctggcctgtgtccc

SEQ ID NO: 876 - DNA, 52 bp, HOMO SAPIENS
gtcttgtttttttttaattttattttttgagatggagtctcctgtcacccag

SEQ ID NO: 877 - DNA, 120 bp, HOMO SAPIENS
gatcaattacctaactgttccttggtctatttatgtataagaatgctttttaaagcacatgtctcattttaaatgacgcacaaactgaagatgttaataaaatttaagagtaatacaatg

SEQ ID NO: 878 - DNA, 126 bp, HOMO SAPIENS
cagagcgaaaaactttattaacaacaggtttcaacgagaaagcaaatgaatacccctagaaacatggacagcatctcccccagcagcaggcatcgtttatggtcggaactacgacggtatctgatc

SEQ ID NO: 879 - DNA, 71 bp, HOMO SAPIENS
tttttggtttttgcagttatttcaattcaggttttattaaagttgtttctgaatattttttctcagtgatc

SEQ ID NO: 880 - DNA, 54 bp, HOMO SAPIENS
gatcgcaccactgcactccactccagcctgggtggcagagtgagactccatctc

SEQ ID NO: 881 - DNA, 256 bp, HOMO SAPIENS
gatccattgatttgtagacctgtgcctcacatgacatactaaccaaccaactatgtttctctgtttgcttcacataatacttaaactaactagactacaacataaaaatataaactgtaaaaaataatagaacttttggaataaatatagttagttggttaatgttagatagaatataaaaagattttgtattttaaaataaatatagttagttggttaatgttagatagaatataagaagattttgtatttgggg

SEQ ID NO: 882 - DNA, 17 bp, HOMO SAPIENS
ccagttgtcaaatgatc

SEQ ID NO: 883 - DNA, 47 bp, HOMO SAPIENS
gtggcactcctgcccagccggcttattagcagttgtatagacagatc

SEQ ID NO: 884 - DNA, 62 bp, HOMO SAPIENS
agtagggacggggtttcaccatgttggttaggctggtcttgaactcccgacctcaagtgatc

SEQ ID NO: 885 - DNA, 274 bp, HOMO SAPIENS
gatcctgaggattacagctgtggaatttttgtccaagcttcaaataattttgaaagaaattttcccatatgaaaaaaggagagaacactggcatctgttgaaatttggaagttctgaaattatagtatttttaaaaattgcactgaagtgtatacacataaagcaggtcttttatccagtgaacaggatgttttgctttagcagcagtgacataaaattccatgttagataagcatatgttacttaccttgttattaaatatttcttgaaaagc

SEQ ID NO: 886 - DNA, 44 bp, HOMO SAPIENS
gatttttgaaactttttatttatattttggtcttacaaatgatc

SEQ ID NO: 887 - DNA, 87 bp, HOMO SAPIENS
gatctcggatgaccaaaccagccttcggagcgttctctgtcctacttctgactttacttgtggtgtgaccatgttcattgtaatctc

SEQ ID NO: 888 - DNA, 102 bp, HOMO SAPIENS
gatcaaaatagtagaaatagttgaagtgtggctttaatcagctgctcagaaaggaaaactgattgtcctctagccagggcttcaggactgggtcaagaaacc

SEQ ID NO: 889 - DNA, 60 bp, HOMO SAPIENS
gatcacgaggtcaggagatggagaacatcctggctaacacagtgaaaccccatctctact

SEQ ID NO: 890 - DNA, 83 bp, HOMO SAPIENS
gggtgatagcttcttgtagtttctgatttattaataaatgagtaatatataaatttttctttgctttttttttttaattgatc

SEQ ID NO: 891 - DNA, 27 bp, HOMO SAPIENS
gatcagaccacaacttttcaatgttta

SEQ ID NO: 892 - DNA, 90 bp, HOMO SAPIENS
gatcttaataatctgctcctgaacttattcagctcagaaaatcatataacaacctgagtacagaatttattttggaaattgtatgaagaa

SEQ ID NO: 893 - DNA, 38 bp, HOMO SAPIENS
gtgttgctgaccttcacttttatttaaatatagtgatc

SEQ ID NO: 894 - DNA, 73 bp, HOMO SAPIENS
gatcaggacacgtatctgtcccatttccagatacatatgaaataacgaagagttattaaattattctgaattt

SEQ ID NO: 895 - DNA, 210 bp, HOMO SAPIENS
ggttcaaacaaattttattgccgttacgtcaaaaacagtaggaaataataacattgaaaaaccaagttcctgctatgtagcattttatacctaggagcttttcttataaaccgtaacataaaatatgatacagcatcacaatataccatatgtaaaatgatactgaattgtacttccatacaattcaaatattaaattattagtatgatc

SEQ ID NO: 896 - DNA, 119 bp, HOMO SAPIENS
gatctaaataagtgtttcaaggaaagttttcctaagcaaatgtaatattacctcatttgggcatcattactctgttaattctatatcaaaggaaataaacttgctacttgcactaaatg

SEQ ID NO: 897 - DNA, 123 bp, HOMO SAPIENS
gttttgaaactttgtttcccatactgttttcagccttttgtttataattagaaattgtgagaagcttcatttagtgtttaaaaatgtggggagataaatcagacttaacatgtatgtaagatc

SEQ ID NO: 898 - DNA, 143 bp, HOMO SAPIENS
gacgctcacacttagtttttattagccacagtttcccacagttttctacctcctaggaaatacacagctcaccaagggcacccagtcaccattctgtcctgcttgcatggctgacactgttgctcaccgagggtgagcagatc

SEQ ID NO: 899 - DNA, 100 bp, HOMO SAPIENS
accttccctgattttctccgtggcattagtggtgaagaggaagaggaggaggaagaagagatgttaatcagtgaagaggagataccattcaaagatgatc

SEQ ID NO: 900 - DNA, 49 bp, HOMO SAPIENS
gatcgggccactgcactccagcctaagcaacagagcaagactacgtctc

SEQ ID NO: 901 - DNA, 68 bp, HOMO SAPIENS
aactatggcttacatttattttaaatttcactaaatacaaatcttgattgtcatgccagttttagatc

SEQ ID NO: 902 - DNA, 23 bp, HOMO SAPIENS
ggggtattattttgttcgagatc

SEQ ID NO: 903 - DNA, 196 bp, HOMO SAPIENS
gatcggtccacgaaagttagcccatatgtatatcttgaatagtataggggagggtattcataaagtccttatgtggttttaactaagtgaaattatggacaagagaaataattgtaaaatcgtcttaaaggaaaatttaatttttactcctgtttatgggacattcgttctattaactgtcaggcacaatttctgt

SEQ ID NO: 904 - DNA, 39 bp, HOMO SAPIENS
cagaattgaaacccatagaacacattagaaatttagatc

SEQ ID NO: 905 - DNA, 286 bp, HOMO SAPIENS
gatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaat

SEQ ID NO: 906 - DNA, 153 bp, HOMO SAPIENS
gatcttatttggaattgacattctctattgtaattttgttcctgtttatttttaaattttctttttgtttcactggaaaggaaagatgatgctcagttttaaacgttaaaagtgtacaagttgctttgttacaataaaactaaatgtgtacac

SEQ ID NO: 907 - DNA, 51 bp, HOMO SAPIENS
gatcttcaagatagaaccacacaggaggagcagctccgacaacagataact

SEQ ID NO: 908 - DNA, 63 bp, HOMO SAPIENS
gatcaagtgagaggatgagggctgaaagcaatcaagctactgtgattggaaccagccggactc

SEQ ID NO: 909 - DNA, 27 bp, HOMO SAPIENS
cataaaaagcacaaaaacaaaaagaag

SEQ ID NO: 910 - DNA, 75 bp, HOMO SAPIENS
aagaagcttcaatattcacttttattatttataaaaaaaattaaggcaccagagtgcatatggtgtactgtgatc

SEQ ID NO: 911 - DNA, 49 bp, HOMO SAPIENS
gatcacagcactgtactccagcctaggtgacggagtgagactctgtctc

SEQ ID NO: 912 - DNA, 58 bp, HOMO SAPIENS; identical to SEQ ID NO: 66

SEQ ID NO: 913 - DNA, 111 bp, HOMO SAPIENS
gatcaataaattataccaaatatatgtttacagtatgatttaaagtctgattcagaccagggactctattttaagttcaactgaaataacactgggttttaattatatcac

SEQ ID NO: 914 - DNA, 87 bp, HOMO SAPIENS
gatctgtttatttcagtatgtagcaaacaatatcttaaagtgtccgattcactacttgttaattaaaaaagttatgattaatgtaaa

SEQ ID NO: 915 - DNA, 25 bp, HOMO SAPIENS
gatccaataaagttgtaaaacaggc

SEQ ID NO: 916 - DNA, 224 bp, HOMO SAPIENS
gagacagagtttcactcttgttgcccaggctggagtgcagtggcacaatctcggctcactgcaacctccgcctcctgggttcaaccaattctcctgccccagcctcctgagtagctgggattacaggcatgtgccaccacgcccagctaattttgtatttttagtagagatggggtttctccatgtttgtcaggctggtctttaactcctgacctcaggtgatc

SEQ ID NO: 917 - DNA, 93 bp, HOMO SAPIENS
aatttgagagcaggtactgtttattaaccaaccagcttagaaaaataatcatggtagacaccttagttcattcttctaataagcctgttgatc

SEQ ID NO: 918 - DNA, 171 bp, HOMO SAPIENS
aaatttcaaaggcaatttaaaactggggggaaatacatttttaaaattttggggttgaagttcactacactatgagactaatttcgtatgtggcaagttgcaactctccaaaggtgtgaataaacatcagaaagcccaaagtgaagagccagtacatcaacatgcatgatc

SEQ ID NO: 919 - DNA, 107 bp, HOMO SAPIENS
acatacacatctaacggtgttgtccatttaattcgatttatatgatttgctgtgccactttgactggcttgcagaacaccccaaattctcttttccccataaagatc

SEQ ID NO: 920 - DNA, 178 bp, HOMO SAPIENS
gttctggaagctttttattgttgctccaagtttaagaattttcactgatggcagaaaaccaacacttttagacaatctcccgcatagccctgcccagcttctgaatcttggtcttgacagacatgtcaacatatttctcgccaatgcgcacaatcattccacccaagattgacggatc

SEQ ID NO: 921 - DNA, 127 bp, HOMO SAPIENS
gatctgggttctgacctatctcttacagtccataatgttgtgattctaagaatatttcattccttactgctttctgccctccattctgaccaatgctgcaaccacaaataaaaacattacttacccc

SEQ ID NO: 922 - DNA, 57 bp, HOMO SAPIENS
gatccccaaagtcaacatatgacattaagccaggcatttcaccttaaaaacaatagt

SEQ ID NO: 923 - DNA, 36 bp, HOMO SAPIENS
gatctacaaggaatttgaaaagaggaagatgctatg

SEQ ID NO: 924 - DNA, 215 bp, HOMO SAPIENS
ggaagcagaaaaaaacagctgttttattcaactattcacaatagagaatatttataatctagtcactgaaacacgcatttcttaggccactcctagaatatgggaatagctgttacataaaatactgttttataattatattaataagaaaaagcattttacctgttccaagaggggaaaaaagctcctcattgagtatcctttgtctctagatc

SEQ ID NO: 925 - DNA, 48 bp, HOMO SAPIENS
gatcgcaccgctgcaccccagcctggcgacagagagagactccgtctc

SEQ ID NO: 926 - DNA, 103 bp, HOMO SAPIENS
gatcatgttgtaggcctgaatacagaaaaatatgctgaaactgtcaaacttaagcataaaagaagcccaggtaaagtaaaagacatatcaattgatgttgaaa

SEQ ID NO: 927 - DNA, 126 bp, HOMO SAPIENS
aaaagaaatggatgcttttattcaacctgagttaaaaccacaatgaatcatatcacagttcatgggaatcataataaatgactagcaaaccactcacaaaatcaaagctatcaacatactgggatc

SEQ ID NO: 928 - DNA, 97 bp, HOMO SAPIENS
gatcatcaactcctgcctgacaaattcccttcaccaacctatttagttgttacacttttacacttgggaacttttcggtatgcatattatatttcac

SEQ ID NO: 929 - DNA, 79 bp, HOMO SAPIENS
gatcgtgccattgtgatatgaatatgccttatatgctgatatgaatatgccttaaaataaagtgttccccacccctgcc

SEQ ID NO: 930 - DNA, 215 bp, HOMO SAPIENS
gatcctgctcttgacagcctcagtcaggccatagcattccagcaagccaaaattgaagaagaacaaaaaaaatggaaacccagttcaggagtcccctcaacatcagatgattcaagacgcccaaggataaagaaaagcacatatttcagtgatgaggaagaacttagtgattaaaatcttgccccagcacagtaataaaaatcaagatttgttag

SEQ ID NO: 931 - DNA, 52 bp, HOMO SAPIENS
gatcactgcagcttctaggacccggtttcttttactgatttaaaaacaaaac

SEQ ID NO: 932 - DNA, 295 bp, HOMO SAPIENS
gggttgcaaagcctttaattagaatgtttgtattttttacatcatgcataacttcacatttgtgattaattagtaattatttcaatacttgtaagctcatctgcctcagatttaatcataatacatgaattaaattaatcaaattaaggaacagcaatttagaaagaaacacactttaagaaatcaaaattctcaattcaggcagtctgtttctatcatttggtattctactcctttaaaaatttcatattgcccaacaaaaagtggttatttttactgtttttggagatgactg

SEQ ID NO: 933 - DNA, 211 bp, HOMO SAPIENS
gcttattacggaaaaacacaccacataattcaactagcaaagaagactgcttcagggcgtgtaaaatgaaaggcttccaggcagttatctgattaaagaacactaagagagggacaaggctagaagccgcaggctgtctacactatagcaggcgctatttgggttggctggaggagctgtggaaaacatggagagattggtgctggagatc

SEQ ID NO: 934 - DNA, 13 bp, HOMO SAPIENS
gatcatactaaaa

SEQ ID NO: 935 - DNA, 223 bp, HOMO SAPIENS
gagacagggcctcactcttgtcgcccaggctggagtgcaacagcacaaactgggcttactgcaacctccgcctcctgggccaaagagattctctggccctaggtcccggaatatcagggaccacaggcacctgccaccatgcccagccagcccttgtattttctgtagagatggggttttgccatgccacccaggctagtcctgaacccccaagctcaagtga

SEQ ID NO: 936 - DNA, 116 bp, HOMO SAPIENS
gggggtaggctctttattagacggttattgctgtactacagggtcagagtgcagtgtaagcagtgtcagaggcccgcgttcagcccaagaatgtggattttctctccctattgatc

SEQ ID NO: 937 - DNA, 53 bp, HOMO SAPIENS
gatcctggggcaacccatctggtctcttgaataaaggtcaaagctggattctc

SEQ ID NO: 938 - DNA, 54 bp, HOMO SAPIENS
gatcgtgccactgtactccagcctgggcgacagagtgagactctgtctcaaatt

SEQ ID NO: 939 - DNA, 112 bp, HOMO SAPIENS
accatttaacttgttttaatgtttcttcacaaatggtgaaaaatactaaagtacagacaaggaataatcataatgttgtggccaacattataaatatggaattataaattta

SEQ ID NO: 940 - DNA, 57 bp, HOMO SAPIENS
gtagagatggagtctatgttgcccaggctggtctcaaactcctgagctcaggtgatc

SEQ ID NO: 941 - DNA, 121 bp, HOMO SAPIENS
gatccagcgacactcttgtatttatctgttctctttttagtcagtcacttcaaaaaaacaaaaaacaaacaaaaaaaagctgtacattttaacataaaataaattatgatgagccattttt

SEQ ID NO: 942 - DNA, 72 bp, HOMO SAPIENS
gatccaatgctattctctaattgtagaccttactcatgtttctccaattgtccttttaacatcctttatagc

SEQ ID NO: 943 - DNA, 70 bp, HOMO SAPIENS
tttatttgtggtatttttctataacaaaacaagtagctctaggaaaagagattttattttgtaaacgatc

SEQ ID NO: 944 - DNA, 187 bp, HOMO SAPIENS
aaaacttgatgtttaaataatataatgtagcaatagtagatatcagattctctctttgccctcttctgtccccagggtctgcaattgctgcttattgtagttgctgggtttgttttttttgtttgtttagtttagtgacttttctgaactaattctgaacagtctgtattcattgttgtgtttgatc

SEQ ID NO: 945 - DNA, 60 bp, HOMO SAPIENS
gatccacggttgtgaccatgtattaccacaaatttaacaataaaaaattgttttaagagt

SEQ ID NO: 946 - DNA, 64 bp, HOMO SAPIENS
tttttatgattgaaagaataaatttattcaattggctccaatgtaatcctattttcaccagatc

SEQ ID NO: 947 - DNA, 22 bp, HOMO SAPIENS
gatcatttttacattaaacgtg

SEQ ID NO: 948 - DNA, 15 bp, HOMO SAPIENS
atggaggctttgatc

SEQ ID NO: 949 - DNA, 47 bp, HOMO SAPIENS
gatcacaccactacactccaacctgggcaacagagccagactctgtc

SEQ ID NO: 950 - DNA, 42 bp, HOMO SAPIENS
gacttagattcagtttattttaattacatatattgtttgatc

SEQ ID NO: 951 - DNA, 96 bp, HOMO SAPIENS
gatctgtaatagcatattgtagatgcactttgcagcagttggaaaagaaagtgttgtgtgatttgattgaaataaaactaaatgtgttgtcctcct

SEQ ID NO: 952 - DNA, 60 bp, HOMO SAPIENS
gatcacgaggtcaggaatttgagactagtgtggccaacatggtgaaaccccatctctact

SEQ ID NO: 953 - DNA, 49 bp, HOMO SAPIENS
gatcatgccactggactccagcctaggtgacagagcgagactctgtctc

SEQ ID NO: 954 - DNA, 93 bp, HOMO SAPIENS
gctggggtcggtactctgggaaacaactcttttagacttttctattaatgccctatgctgttccatgtccttatatactatctgtgttggatc

SEQ ID NO: 955 - DNA, 68 bp, HOMO SAPIENS
gatcattctgagtgtgcgagtgtgtgtgcacatgttacaaaggcaactaccatgttaataaaatattc

SEQ ID NO: 956 - DNA, 113 bp, HOMO SAPIENS
gatcattcactgtttttgttttaaaactggtcagtactatgaagcttctgtggtttgtatgatgttttaactttcactttaaactgcatagaataaattaaattgaaaacaac

SEQ ID NO: 957 - DNA, 53 bp, HOMO SAPIENS
gatcaccggaggttaggaattcaagactgaaaccttgtctctactgaaaatac

SEQ ID NO: 958 - DNA, 49 bp, HOMO SAPIENS
gagacggagtcttgctctgtcacccaggctggagtacaatggcgtgatc

SEQ ID NO: 959 - DNA, 100 bp, HOMO SAPIENS
gatcaaacctttctggcctgttatgattctgaacatttgacttgaaccacaagtgaatctttctcctggtgactcaaataaaagtataatttttacctgc

SEQ ID NO: 960 - DNA, 62 bp, HOMO SAPIENS
gatcttacaataatgttccactctgcaaatttttaaggttcaaataaagtttaattgtttgc

SEQ ID NO: 961 - DNA, 73 bp, HOMO SAPIENS
gatcccagggacctcagtcggcttaatcagagtgtggcatagaagctatttaatgattaaagtcatttgcagt

SEQ ID NO: 962 - DNA, 119 bp, HOMO SAPIENS
gagcccaaacaccaaagatggaaggtgaaaataaagactgcgtgaccgggaagaaagtttgaattactaatagtggggaataataatttcagttttggttttaacattctggaattcct

SEQ ID NO: 963 - DNA, 222 bp, HOMO SAPIENS
gatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccac

SEQ ID NO: 964 - DNA, 50 bp, HOMO SAPIENS
gatcgcaccactatactccagcctgggtgacagggcaagactccttctcc

SEQ ID NO: 965 - DNA, 73 bp, HOMO SAPIENS
gctgttccttgactgttagttgggccttggcctatgtcatacccatgggctgccacatgggggtggagggatc

SEQ ID NO: 966 - DNA, 49 bp, HOMO SAPIENS
gatcgcgccattgcactgcagcctaggtgacagagcgagactctgtctc

SEQ ID NO: 967 - DNA, 108 bp, HOMO SAPIENS
acaaattttaacatattttattgatttaggggccaaacaaggcagcatttggtcagttaagaaaggcacctcattgaaaataatacatcacagtgctgttgagtgatc

SEQ ID NO: 968 - DNA, 21 bp, HOMO SAPIENS
gatcgaacaagaccctgtctc

SEQ ID NO: 969 - DNA, 145 bp, HOMO SAPIENS
gtagaccacaaaatccacttttatttggtagtttttacatctatggccaaatagtattgggttgacacagtaatctagagtttacctctgaaaaaaagtaatgtgagtctgtctaaccatattttcctaactcaacagtaggatc

SEQ ID NO: 970 - DNA, 53 bp, HOMO SAPIENS
gatctcattgatgtacacaaccaagttccaataaagtgctagaatgtgcaaac

SEQ ID NO: 971 - DNA, 37 bp, HOMO SAPIENS
gatccaaggtttttgttcaatacaactatttaatctc

SEQ ID NO: 972 - DNA, 149 bp, HOMO SAPIENS
gatctcaatcttcggggtgtgatgaatagcgaatcatctcaaatccttgagcactcagtctagtgaagatgttgtcattatgtacaatacataactagtttaattaactatgtgatgttaactattattaataaattttaacattttcc

SEQ ID NO: 973 - DNA, 156 bp, HOMO SAPIENS
gatcctgtgtttgcaactggggagacagaaactgtggttgatagccagtcactgccttaagaacatttgatgcaagatggccagcactgaacttttgagatatgacggtgtacttactgccttgtagcaaaataaagatgtgcccttattttacct

SEQ ID NO: 974 - DNA, 119 bp, HOMO SAPIENS
attaggaaattgtagataattttaatgaagtcaacgtcaagaatataatctggttgtaataccaatacaatgtgttctaaaataatttttctattgagttaaatattcatctgtagatc

SEQ ID NO: 975 - DNA, 215 bp, HOMO SAPIENS
aggtcaaggtgagtttattgtccaaatagcataacctaattgcattcaaaaccattttcaaatccatctttaaactagtcagaaaacaggttattatttttttaaatcacttaacactgaacagataagacctcttaaaaggcagctgactatatcatgtcaccatcatagccaatacaacatttttgccatacttcctaaaaaccttttcgcat

SEQ ID NO: 976 - DNA, 153 bp, HOMO SAPIENS
gatctttgttcttgacctctagaccccaagatgtgaacagtgcacgtgttaatgtcatctttgctcatgtgttataagccccaagttgctgtatattttcacaagtatgtctacacactggtcatgattttgataataaataacgataaatcg

SEQ ID NO: 977 - DNA, 36 bp, HOMO SAPIENS
gatccagaacataaggggcctaaagagagaggaagc

SEQ ID NO: 978 - DNA, 99 bp, HOMO SAPIENS
atcagagtcccatacctcacgggtattttctcaatcagtgaacacctcaagtactagcccatgtgttttgagtaaaaagggctcctttcaacgaggatc

SEQ ID NO: 979 - DNA, 66 bp, HOMO SAPIENS
ggttaaagtgctgatgtcatttaattagattgtttacaaatttaacaggatggctacaaagagatc

SEQ ID NO: 980 - DNA, 41 bp, HOMO SAPIENS
tgaccattggcacacgctagaagtttatggcagagctttac

SEQ ID NO: 981 - DNA, 141 bp, HOMO SAPIENS
gatcttagagctaaaaaggactgtaaaaattacccagaacagcgtcctcagacttaaccttctgcaagttatgtctgtatataagaagattctaattgctaactgtttatacttttctgaataaaatagttgtttctaatt

SEQ ID NO: 982 - DNA, 120 bp, HOMO SAPIENS
ttttttatttattgagggcaaggggatgcacacaatacaaaaatcaaaagcttatctggtatttaacttttctttctctgcttgtcaaatgagagttagattttatttttacatttgcta

SEQ ID NO: 983 - DNA, 142 bp, HOMO SAPIENS
gatctgacccccagcatgattgtatttctttcccctccagtcaaaccgtaatacagtttccagaaaatttgacagtctgcaatgccaaaagggtaaaaatctgtatttcacagttgtatagaataaaggctttagttaaaat

SEQ ID NO: 984 - DNA, 131 bp, HOMO SAPIENS
gatcccaaatttgaagtcatcgaaaaaccccaggcctgaagaaataaagtaaaattaatctggtaatttgtcacggattagttgtacaactagttagaagtttcagaataaacatgcatttcataactgtc

SEQ ID NO: 985 - DNA, 216 bp, HOMO SAPIENS
gatccacatctcaaagaagtggggcttcaccaagttcaatgccgatgaatttgaagacatggtggctgaaaagcggctcatcccagatggctgtggggtcaagtacatccccagtcgtggccctctggacaagtggcgggccctgcactcatgagggcttccaatgtgctgcccccctcttaatactcaccaataaattctacttcctgtccaccc

SEQ ID NO: 986 - DNA, 51 bp, HOMO SAPIENS
cccttttccatttgaaagaaaggactctggggtttttactgttacctgatc

SEQ ID NO: 987 - DNA, 107 bp, HOMO SAPIENS
gatctccagtggctcttggcgggaccggcccttcaagccctacaacttcttggccctgaggagacctgtgggccacctattgtctaataaagtgggcagttgccccc

SEQ ID NO: 988 - DNA, 171 bp, HOMO SAPIENS
gatccggaaatatggcctcaatatgtgccgccagtgtttccgtcagtacgcgaaggatatcggtttcattaagttggactaaatgctcttccttcagaggattatccggggcatctactcaatgaaaaaccatgataattctttgtatataaaataaacatttgaaaaaac

SEQ ID NO: 989 - DNA, 151 bp, HOMO SAPIENS
attcggctcagtctaatcctttttgtagtcactcataggccagacttagggctaggatgatgattaataagagggatgacataactattagtggcaggttagttgtttgtagggctcatggtaggggtaaaaggagggcaatttctagatc

SEQ ID NO: 990 - DNA, 110 bp, HOMO SAPIENS
gatccccgcaggtggcacgtgacagctagggttcaaaacgttctcaccaaatccaatgctcctcacatattaattttataaccagacaaataaatattagagacaaccac

SEQ ID NO: 991 - DNA, 51 bp, HOMO SAPIENS
gatcatgctgctgtactctagcctgggcaatgagagtgaaactcttatctc

SEQ ID NO: 992 - DNA, 125 bp, HOMO SAPIENS
gatcctagtgcttaaattaatgttagcagggattaacatgtaaaatgttccttttaaagtcactaaatctcaattttttacaaaaaagtttttcatttttttaaatggtagaataaagtcttagc

SEQ ID NO: 993 - DNA, 119 bp, HOMO SAPIENS
tacctcgttgcactgctgagagcaagatgggtcaccagcagctgtactggagccacccgcgaaaattcggccagggttctcgctcttgtcgtgtctgttcaaaccggcacggtctgatc

SEQ ID NO: 994 - DNA, 309 bp, HOMO SAPIENS
gatcatagctcctagtgaataacatgaggtgttttaaagttaggtctattacagaattaacataagcacaattttaattttatgatatctgatatgcctatctaatacatcttaatgcattaaatgctgcagataagaatcttgctatttttcaagtagacaacattagaaagttgaatcatgaactcattcaattcatcaagtcaaaagcactgagtgttttgctagtctagtgacatgggttattgtatttccgagaagtgcctctcatttctggggagttgtttacatataccctgcatttaaacc

SEQ ID NO: 995 - DNA, 104 bp, HOMO SAPIENS
ataaaatcctttattttatgagtggcaatcgtcacctccctacccaacacataaacacacatacccatctagtctttgttttcatcacatttcccaaactgatc

SEQ ID NO: 996 - DNA, 136 bp, HOMO SAPIENS
atacctacaatttacttaatgttccttttattaaaacgcgtggttcatgaacaactacagactgaatccagattattacctgttgctttcagtattttcgtgatggcttttaatcttatgaaatcatcttgagatc

SEQ ID NO: 997 - DNA, 64 bp, HOMO SAPIENS
gatcctaatgtaaacattgtacctaaagatggcacagggtgggcatcaaaacaagagcaacatg

SEQ ID NO: 998 - DNA, 138 bp, HOMO SAPIENS
gatcacaagtgcagcattccttaattccttctgctatatgtcacacagttgttatttggagaaccaagtatgtattgcatgaaaacattatgacttttttctcttagtttaaataaactccgaggtaactggacttct

SEQ ID NO: 999 - DNA, 246 bp, HOMO SAPIENS
ccaagttggtccgagtgcaagctggtaaaagagattttttttccaaacacaaacaagaacatgtattccaaagacaatgaatataatactgataaaaacacaaacatagtgcttaagaaaagatggggtgaggtggagatgtagattgggcaggaacaattttctagtaaataaggagctgcagtctttagaggcaacatggtatgctagaaaggacactggaataagaatcagaagacctggatc

SEQ ID NO: 1000 - DNA, 48 bp, HOMO SAPIENS
gatcacaccactgcactccagtcagagcaacagagtgagactcaatct

SEQ ID NO: 1001 - DNA, 139 bp, HOMO SAPIENS
gaaattgaatttttattccaaatgactaatatctagaaaggcagtagctaacactcaaaacaatttgtttaagtgtaaattaaaagcagttgatttgcaggagagcaagcagtggggtagtggccatggcactctgatc

SEQ ID NO: 1002 - DNA, 92 bp, HOMO SAPIENS
gatcctgagctaggagagtacattttgcagacctttggtttccatattaagaaattcagatttttatgtacaataaagaagttctggaattc

SEQ ID NO: 1003 - DNA, 124 bp, HOMO SAPIENS
aaatttgaccaaaaaaaatttattgtacaattacccaccactggatttgactcagagaggacccccagagggtgtctccatcttccctatttattttcagcccttgagggcttcattgtagatc

SEQ ID NO: 1004 - DNA, 168 bp, HOMO SAPIENS
gatccggttggactctgacatcggatgccctcaaacatacagaacttccaaactcaagtccagccataagctattttgccaacatgtcagagtaatctgtatttttgtatgtgatttctacttttatagacttgttttaaaacaataaaacacatttttataaaaatg

SEQ ID NO: 1005 - DNA, 41 bp, HOMO SAPIENS
cttctcaaacccttgtgtcgagggctgactttcaatagatc

SEQ ID NO: 1006 - DNA, 62 bp, HOMO SAPIENS
aaaaatcaaactgtttattgttgctttttgccatttggtagcattttacacaggcttcgatc

SEQ ID NO: 1007 - DNA, 94 bp, HOMO SAPIENS
actgagtgttggagttttgctctgtcacccaggttgaagtacaatggcataggcatagttcactgcagccttgaatttctgggctccagtgatc

SEQ ID NO: 1008 - DNA, 47 bp, HOMO SAPIENS
gttagtctttccactcggatgttgaggtcctctgcaggtctctgatc

SEQ ID NO: 1009 - DNA, 135 bp, HOMO SAPIENS
aagacagggaatactttattcaaaacccatcacagaaatggacagcttgggtctgtaacaaagcattcatgttttagagcataggtcagtaattgtatatgagagcatacactgctacatacaaattaactgatc

SEQ ID NO: 1010 - DNA, 54 bp, HOMO SAPIENS
gatgaattctggtgatattttatttagaaaaagataatcactgattttaagatc

SEQ ID NO: 1011 - DNA, 89 bp, HOMO SAPIENS
aacaatgactctttataaacttgtatggggtctaaattatcccttgtaatatatatgtgtgtattcattccacagagaattagatgatc

SEQ ID NO: 1012 - DNA, 151 bp, HOMO SAPIENS
gatctgggagtgctggacgtcatcttccaccccacccagccgcgggtcttctcctcgggggcagacgggactgtccgcctcttcacctagctgttctgcccgcctggggctggggtggtcgtgctgaagtcaacagagcctttaccctgtg

SEQ ID NO: 1013 - DNA, 88 bp, HOMO SAPIENS
cctggcccttcctcagatggttgagctggagctacttttcccccaccacttgtaggggactgctgctctgtgtctgagctctgagatc

SEQ ID NO: 1014 - DNA, 49 bp, HOMO SAPIENS
gatcacactactgcacttcagtacagatggcaagtgacactccatctct

SEQ ID NO: 1015 - DNA, 86 bp, HOMO SAPIENS
gaatttgcaaagcagtttattgcagggttcacaaggcacttcagctcaggaaagctgaaggggtatggagactgatgaggacagat

SEQ ID NO: 1016 - DNA, 58 bp, HOMO SAPIENS
gatcatgccactgcactccggcctaggtgacagagggaaactccatctccaggaaaag

SEQ ID NO: 1017 - DNA, 83 bp, HOMO SAPIENS
gatcacccccaggaggggaaaatggtggcaatgccttttatatattatgtttttactgaaattaactgaaaaaatatgaaacc

SEQ ID NO: 1018 - DNA, 100 bp, HOMO SAPIENS
gatctatttttcctcatctgtaaaataggtgtaataataacaactactttgtcggttgctctgagggttaaatgaaaataaaaagaaaatgtgaaacagc

SEQ ID NO: 1019 - DNA, 71 bp, HOMO SAPIENS
gagatttttagtagagatgggatttcaccatgttggccagactggtttcaaactcctgacctcaagcgatc

SEQ ID NO: 1020 - DNA, 110 bp, HOMO SAPIENS
ggagttgaaacagtttacatgaaggcaatttattaacagaaaatattttgaggaatcttgttcacagacggcgaccacggcgaccccccttcctgcgagtgctgtcagag